(19) United States
(12) Patent Application Publication
(10) Pub. No.: US 2024/0182470 A1
(43) Pub. Date: Jun. 6, 2024

(54) KRASG12C MUTANT PROTEIN INHIBITOR, PREARATION AND USE THEREOF

(71) Applicant: YA THERAPEUTICS INC, Shanghai (CN)

(72) Inventors: Yonghong LIANG, Shanghai (CN); Zhiyong XU, Shanghai (CN)

(21) Appl. No.: 18/547,413

(22) PCT Filed: Mar. 7, 2022
(86) PCT No.: PCT/CN2022/079581

§ 371 Date: Aug. 22, 2023

(30) Foreign Application Priority Data
Mar. 15, 2021 (CN) 202110276073.3
Apr. 7, 2021 (CN) 202110374457.9
May 7, 2021 (CN) 202110496041.4
Jun. 17, 2021 (CN) 202110672205.4

(51) Int. Cl. C07D 471/04; A61K 31/496; A61K 31/517; A61K 31/519; A61P 35/00; C07D 487/04; C07D 519/00

(52) U.S. Cl. CPC C07D 471/04; A61K 31/496; A61K 31/517; A61K 31/519; A61P 35/00; C07D 487/04; C07D 519/00

(57) ABSTRACT

A KRAS^{G12C} mutant protein inhibitor, as shown by formula (I), a composition containing the inhibitor and pharmaceutically acceptable salt, syntheses, intermediates, formulations, and the use thereof. The compounds of the invention have good activity and safety in inhibiting tumor growth.

### CROSS-REFERENCE TO RELATED APPLICATIONS

This application claims priority to International Application Nos. PCT/CN, filed on, the content of each of which is incorporated herein by reference in its entirety.

### BACKGROUND OF THE INVENTION

The present invention relates to novel compounds as inhibitor, process of preparation thereof, and to the use of the compounds in the preparation of pharmaceutical compositions for the therapeutic treatment of disorders in humans, for example cancer.

RAS represents a population of 189 amino acid monomeric globular proteins (21 kDa molecular weight) that are associated with the plasma membrane and bind to GDP or GTP, and RAS acts as a molecular switch. When the RAS contains bound GDP, it is in a stationary or closed position and is “inactive.” When cells are exposed to certain growth-promoting stimuli, RAS is induced to exchange their bound GDP for GTP. In the case of binding to GTP, RAS is “opened” and is capable of interacting with other proteins (its “downstream targets”) and activating the proteins. The RAS protein itself has an inherently low ability to hydrolyze GTP back to GDP, thereby turning itself into a closed state. Closing RAS requires an exogenous protein called GTPase activating protein (GAP) that interacts with RAS and greatly accelerates the conversion of GTP to GDP. Any mutation in RAS that affects its ability to interact with GAP or convert GTP back to GDP will result in prolonged protein activation, and thus conduction to the cell to inform its signaling of continued growth and division. Since these signals cause cell growth and division, over-activated RAS signaling can ultimately lead to cancer.

Structurally, the RAS protein contains a G domain responsible for the enzymatic activity of RAS-guanine nucleotide binding and hydrolysis (GTPase reaction). It also contains a C-terminal extension called the CAAX cassette, which can be post-translationally modified and responsible for targeting the protein to the membrane. The G domain is approximately 21-25 kDa in size and contains a phosphate binding ring (P-ring). The P-loop represents a pocket of a binding nucleotide in a protein, and this is a rigid portion of a domain with conserved amino acid residues necessary for nucleotide binding and hydrolysis (glycine 12, threonine 26 and lysine 16). The G domain also contains a so-called switch I region (residues 30-40) and a switch II region (residues 60-76), both of which are dynamic parts of the protein, since the dynamic portion is converted between stationary and loaded states. The ability is often expressed as a “spring loaded” mechanism. The primary interaction is the hydrogen bond formed by threonine-35 and glycine-60 with the gamma-phosphate of GTP, which maintains the active conformation of the switch 1 region and the switch 2 region, respectively. After hydrolysis of GTP and release of phosphate, the two relax into an inactive GDP conformation.

The most notable members of the RAS subfamily are HRAS, KRAS and NRAS, which are primarily involved in many types of cancer. Mutation of any of the three major isoforms of the RAS gene (HRAS, NRAS or KRAS) is one of the most common events in human tumor formation. Approximately 30% of all tumors in human tumors were found to carry some mutations in the RAS gene. It is worth noting that KRAS mutations were detected in 25%-30% of tumors. In contrast, the rate of carcinogenic mutations in NRAS and HRAS family members was much lower (8% and 3%, respectively). The most common KRAS mutations were found at residues G12 and G13 in the P-loop as well as at residue Q61.G12C is a frequently occurring KRAS gene mutation (glycine-12 is mutated to cysteine). This mutation has been found in about 13% of cancers, about 43% in lung cancer, and almost 100% in MYH-associated polyposis (familial colon cancer syndrome).

As a frontier target, KRAS^{G12C} mutant protein has attracted extensive attention. Araxes (a subsidiary of Wellspring) developed the ARS-853 and ARS-1620 compounds in 2013 and 2016, respectively. In recent years, it has also filed a number of patents for KRAS^{G12C} inhibitors, such as W02016164675 and W02016168540. The ARS-853 compounds show good cell viability, but their pharmacokinetic properties are poor and are not suitable for evaluating the pharmacodynamics of animal models in vivo. ARS-1620 is highly efficient and selective against KRAS^{G12C}, enabling rapid and sustained target action in vivo to induce tumor regression. This study provides in vivo evidence that ARS-1620 represents a new generation of KRAS^{G12C}-specific inhibitors with significant therapeutic potential. Wellspring announced that the FDA has approved ARS-3248 for IND application. Other candidates for KRAS^{G12C} inhibitors include Mirati's MRTX-849 and Boehringer Ingelheim's Bi-2852.

Accordingly, while progress has been made in this field, there remains a need in the art for improved compounds and methods for treatment of cancer, for example by inhibition of KRAS, HRAS or NRAS. The present invention fulfills this need and provides further related advantages.

In brief, the present invention provides compounds, including stereoisomers, pharmaceutically acceptable salts, tautomers and prodrugs thereof which are capable of modulating G12C mutant KRAS, HRAS and/or NRAS proteins. In some instances, the compounds act as electrophiles which are capable of forming a covalent bond with the cysteine residue at position 12 of a KRAS, HRAS or NRAS G12C mutant protein. Methods for use of such compounds for treatment of various diseases or conditions, such as cancer, are also provided.

### SUMMARY

The present invention includes compounds of Formula (I) and pharmaceutically acceptable salts or stereoisomer thereof as provided below and further defined herein:

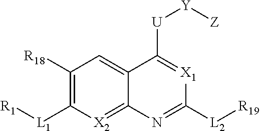

Wherein:
each L₁ at each occurrence is independently selected from bond [symbol] O [symbol] NH [symbol] C1-6alkyl [symbol] CO [symbol] OC₁₋₆ alkyl, NHC₁₋₆ alkyl or S;
each R₁ at each occurrence is independently selected from phenyl, naphthyl, 5-membered heteroaryl, 6-membered heteroaryl, 7-membered heteroaryl, 8-membered heteroaryl, 9-membered heteroaryl, or 10-membered heteroaryl Heteroaryl, each heteroaryl independently at each occurrence containing 1, 2, 3 or 4 heteroatoms selected from N, O, or S; each R1 at each occurrence independently optionally substituted or unsubstituted with 1, 2, 3, 4, 5 or 6 R₂₀;
each of R₂₀ at each occurrence is independently selected from deuterium, halogen, oxo, —C₁₋₆alkyl, —C₁₋₆alkylene-(halo)₁₋₃, heteroC₁₋₆alkyl, —CN, —OR₆, —C₁₋₆alkylene-(OR₆)₁₋₃, —O——C₁₋₆alkylene-(halo)₁₋₃, —SR₆, —S—C₁₋₆alkylene-(halo)₁₋₃, —NR₆R₇, —C₁₋₆alkylene-NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆carbocyclic; each of R₁₂ which is independently and optionally substituted or unsubstituted with 1, 2, 3, 4, 5 or 6 substituents selected from deuterium, halogen, oxo, —C₁₋₆alkyl, —C₁₋₆alkoxy, —CN, —OR₆, —NR₆R₇, —C(═O)R₆, —C(═O)OR₆, —OC(═O)R₆, —C(═O)NR₆R₇, —NR₆C(═O)R₇, —S(O)₂NR₆R₇;
each X₁, X₂ is independently selected from N, CR₂₁ at each occurrence;
each R₁₈ is independently selected from H, D, cyano, halogen, C₁₋₆ alkyl, COOH, NHCOH, CONH₂, OH or —NH₂;
each R₂₁ is independently selected from H, D, cyano, halogen, C₁₋₆ alkyl, COOH, NHCOH, CONH₂, OH or —NH₂;
Each L₂ at each occurrence is independently selected from bond, OC₀₋₆ alkyl, NHC₀₋₆ alkyl, C₁₋₆ alkyl, COC₀₋₆ alkyl or SC₀₋₆ alkyl;
each R₁₉ is independently selected from

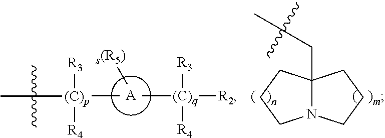

each ring A is a C₃₋₁₀ carbocyclic ring, and the

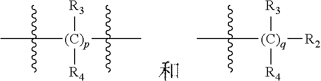

can be attached to the same carbon atom or to a different atom of the ring A;
each R₂ is —OR₆, —NR₆R₇, —SR₆, —S(═O)R₆, —S(═O)₂R₆, 5-10 membered heteroaryl or 3-10 membered heterocyclyl, each heterocyclyl and heteroaryl at each occurrence independently comprising 1, 2, 3 or 4 heteroatoms selected from N, O, S, S═O or S(═O)₂, each R₂ at each occurrence independently and optionally substituted or unsubstituted with 1, 2, 3, 4, 5 or 6 R₂₂;
Each R₃ and R₄ at each occurrence is independently selected from deuterium, hydrogen, halogen, —C₁₋₆ alkyl, —C₂₋₆ alkenyl, —C₂₋₆ alkynyl, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇ or —C₃₋₁₀ carbocyclyl, each heterocyclyl and heteroaryl at each occurrence independently comprising 1, 2, 3 or 4 heteroatoms selected from N, O, S, S═O or S(═O)₂; each R₃ and R₄ at each occurrence is independently and optionally substituted or unsubstituted; 1, 2, 3, 4, 5 or 6 substituents selected from deuterium, halogen, oxo, —C₁₋₆ alkyl, —C₁₋₆ alkoxy, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇;
each R₅ at each occurrence is independently selected from deuterium, halogen, oxo, —C₁₋₆ alkyl, —C₁₋₆ alkylene-(halogen)₁₋₃, C₁₋₆ heteroalkyl, —CN , —OR₆, —C_{1,6} alkylene-(OR₆)₁₋₃, —O—C₁₋₆ alkylene-(halogen)₁₋₃, —SR₆, —S—C₁₋₆ alkylene-(Halogen) ₁₋₃, —NR₆R₇, —C₁₋₆ alkylene —NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl, each heterocyclyl and heteroaryl at each occurrence independently and optionally contains 1, 2, 3 or 4 heteroatoms selected from N, O, S, S═O or S(═O)₂, each R₃ and R₄ at each occurrence independently and optionally substituted or unsubstituted with 1, 2, 3, 4, 5 or 6 substituents selected from deuterium, halogen, oxo, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇.
each R₆ and R₇ is independently selected at each occurrence from hydrogen, deuterium or —C₁₋₆ alkyl, and each R₆ and R₇ is independently and optionally substituted or not substituted with 1, 2, 3, 4, 5 or 6 R₂₂; or R₇ and R₇ together with the N atom to which they are connected together form a 3-10-membered heterocycle, and the 3-10-membered heterocycle may further comprise 1, 2, 3 or 4 heteroatoms selected from N, O, S, S(═O) or S(═O)₂, and the described 3-10 membered heterocycle is independently and optionally substituted or unsubstituted with 1, 2, 3, 4, 5 or 6 R₂₂.
each R₂₂ at each occurrence is independently selected from deuterium, halogen, oxo, —C₁₋₆alkyl, —C₁₋₆alkylene-(halogen)₁₋₃, C₁₋₆heteroalkyl, —CN , —O—C₁₋₆ alkyl, —C₁₋₆ alkylene-(O—C₁₋₆ alkyl ₁₋₃, —O—C₁₋₆ alkylene-(halogen) ₁₋₃, —S—C₁₋₆ alkyl, —S—C₁₋₆ alkylene-(halogen) ₁₋₃, —N—C₁₋₆ alkyl-C₁₋₆ alkyl, —C₁₋₆ alkylene-NC₁₋₆ alkyl base C₁₋₆ alkyl, —C(═O)C₁₋₆ alkyl, —C(═O)OC₁₋₆ alkyl, —OC(═O)C₁₋₆ alkyl, —C(═O)NC₁₋₆ alkyl C₁₋₆ alkyl, —NC₁₋₆ alkyl C(═O)C₁₋₆ alkyl, —S(O)₂NC₁₋₆ alkyl C₁₋₆ alkyl or —C₃₋₆ carbocyclyl;
s is selected from 0, 1, 2, 3, 4, 5 or 6;
p is selected from 0, 1, 2, 3, 4, 5 or 6;
q is selected from 0, 1, 2, 3, 4, 5 or 6;
m is selected from 1, 2, 3;
n is selected from 1, 2, 3
U is independently selected from —C₀₋₄ alkyl-, —CR₈R₉-, —C₁₋₂ alkyl(R₈)(OH)—, —C(O)—, —CR₈R₉O-, —OCR₈R₉-, —SCR₈R₉-, —CR₈R₉S—, —NR₈—, —NR₈C(O)—, —C(O)NR₈—, —NR₈C(O)NR₉—, —CF₂—, —O—, —S—, —S(O)ₘ—, —NR₈S(O)ₘ—, —S(O)ₘNR₈—;
Y is absent or selected from 3-8 membered cycloalkyl, 3-8 membered heterocycloalkyl, 5-12 membered fused alkyl, 5-12 membered fused heterocyclyl, 5-12 membered spirocyclyl, 5-12 membered Membered spiroheterocyclyl, aryl or heteroaryl, each heterocycloalkyl, fused heterocyclyl, spiroheterocyclyl, heteroaryl independently at each occurrence contains 1, 2, 3 or 4 options A heteroatom from N, O, or S, wherein the cycloalkyl, heterocycloalkyl, spirocyclyl, fused ring, fused heterocyclyl, spiroheterocyclyl, aryl, or heteroaryl group is optionally replaced by one or more G¹;
Z is independently selected from cyano, —NR₁₀CN,

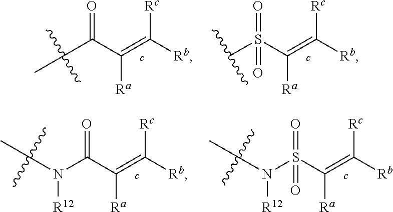

the bond c is a double bond or a triple bond;
when c is a double bond, R^{a}, R^{b} and R^{c} are each independently selected from H, deuterium, cyano, halogen, C₁₋₆ alkyl, C₃₋₆ cycloalkyl or 3-6 membered heterocyclyl. wherein the alkyl, cycloalkyl and heterocyclyl groups are optionally substituted with one or more G²;
R^{a} and R^{b} or R^{b} and R^{c} optionally together with the carbon atoms to which they are attached optionally form a 3-6 membered ring containing heteroatoms;
when the bond c is a triple bond, R^{a} and R^{c} are absent, and R^{b} is independently selected from H, deuterium, cyano, halogen, C₁₋₆ alkyl, C₃₋₆ cycloalkyl or 3-6 membered heterocyclyl replaced by one or multiple G³;
R₁₀ is independently selected from H, deuterium, C₁₋₆ alkyl, C₃₋₆ cycloalkyl or 3-6 membered heterocyclyl, wherein the alkyl, cycloalkyl and heterocyclyl are optionally replaced by one or more G⁴;
G¹, G², G³ and G⁴ are each independently selected from deuterium, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR₁₁, —OC(O)NR₁₁R₁₂, —C(O)OR₁₁, —C(O)NR₁₁R₁₂, —C(O)R₁₁, —NR₁₁R₁₂, —NR₁₁C(O)R₁₂, —NR₁₁C(O)NR₁₂R₁₃, —S(O)ᵢR₁₁ or —NR₁₁S(O)ᵢR₁₂, wherein the described alkyl, alkenyl, alkynyl, cycloalkyl, heterocycloalkyl, aryl, Heteroaryl is optionally replaced by 1 or more deuterium, cyano, halogen, C₁₋₆ alkyl, C₂₋₆ alkenyl, C₂₋₆ alkynyl, C₃₋₈ cycloalkyl or 3-8 membered heterocycle base, C₆₋₁₀ aryl, 5-10 membered heteroaryl, —OR₁₄, —OC(O)NR₁₄R₁₅, —C(O)OR₁₄, —C(O)NR₁₄R₁₅, —C(O)R₁₄, —NR₁₄R₁₅, —NR₁₄C(O)R₁₅, —NR₁₄C(O)NR₁₅R₁₆, —S(O),R₁₄ or —NR₁₄S(O)ᵢR₁₅ substituent;

R₈, R₉, R₁₁, R₁₂, R₁₃, R₁₄, R₁₅ and R₁₆ are each independently selected from hydrogen, deuterium, cyano, halogen, C₁₋₆ alkyl, C₃₋₈ cycloalkyl or 3-8 membered monocyclic heterocyclyl, monocyclic heteroaryl or phenyl;
and i is 1 or 2.

In some embodiments,

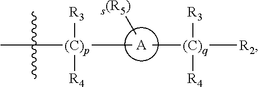

each Ring A of is a 3-membered carbocycle, a 4-membered carbocycle, a 5-membered carbocycle, or a 6-membered carbocycle, and the described

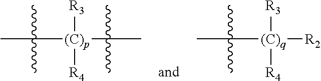

can be attached to the same carbon atom or on a different atom of the ring A; each R₂ is at each occurrence independently selected from —NR₆R₇ or a 3-6 membered heterocyclyl, each heterocyclyl at each occurrence independently contains 1 heteroatom selected from N atom, each R₂ at each occurrence is independently and optionally substituted or unsubstituted with 1, 2, 3, 4, 5 or 6 R₂₀.

In some embodiments, R₆ and R₇ in each R₂ are independently selected at each occurrence from hydrogen, deuterium, methyl, ethyl, propyl, or isopropyl; or R₆ and R₇ in R₂ together with the N atom to which they are attached together form a 3-6 membered heterocycle, the 3-6 membered heterocycle may further comprise 1 heteroatom selected from N, and the 3-6 membered heterocycle Heterocycles are independently optionally substituted or unsubstituted with 1, 2, 3, 4, 5 or 6 R₂₀.

In some embodiments, each R₂ is independently selected at each occurrence from —NH₂, —N(CH₃)₂, —N(CH₃)(CH₂CH₃), —N(CH₂CH₃)₂,

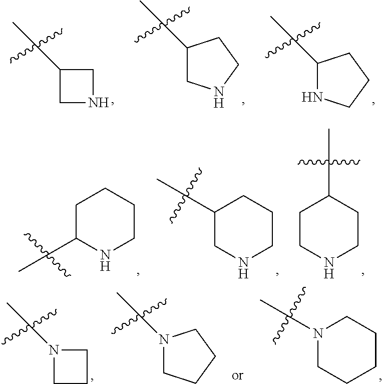

each R₂ independently and optionally substituted or unsubstituted with 1, 2, 3, 4, 5 or 6 R₂₀.

In some embodiments, each R₂ at each occurrence is independently selected from —NH₂, —N(CH₃)₂, —N(CH₃)(CH₂CH₃), —N(CH₂CH₃)₂,

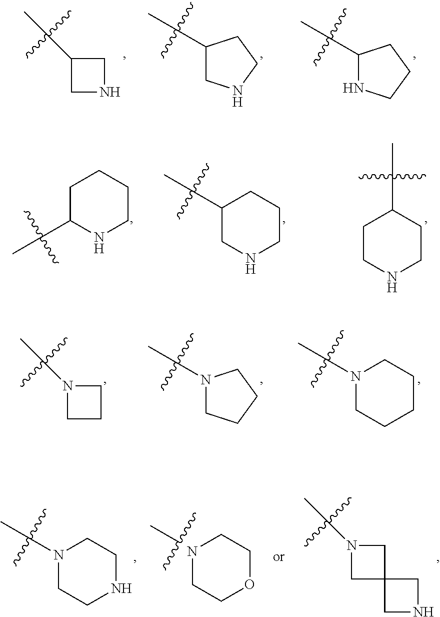

each R₂ independently and optionally substituted or unsubstituted with 1, 2, 3, 4, 5 or 6 R₂₀.

In some embodiments, each R₂₀ at each occurrence is independently selected from deuterium, halogen, oxo, —C₁₋₆alkyl, —C₁₋₆alkylene-(halogen)₁₋₃, C₁₋₆ Heteroalkyl, —CN, —OR₆, —C₁₋₆ alkylene-(OR₆)₁₋₃, —O—C₁₋₆ alkylene-(halogen)₁₋₃, —SR₆, —S—C₁₋₆ alkylene-(halogen)₁₃, —NR₆R₇, —C₁₋₆ alkylene-NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl; each R₁₂ is independently and optionally substituted or unsubstituted with 1, 2, 3, 4, 5 or 6 substituents selected from deuterium, halogen, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇.

In some embodiments, each R₂₀ at each occurrence is independently selected from -deuterium, —F, —Cl, —Br, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CH₂F₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —OCH₂F, —OCHF₂, —OCF₃, —OOOF, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SOF, —SCHF₂, —SCF, —SCH₂CH₂F, —SCH₂CH₂F₂, —SCH₂CF₃, —SCH₂CH₂CH₂F, —SCH₂CH₂CHF₂, —SCH₂CH₂CF₃, —NH₂, —NHCH₃, —NHCH₂CH₃, —NHCH₂CH₂CH₃, —NHCH(CH₃)₂. —N(CH₃)₂, —N(CH₃)CH₂CH₃, —N(O)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)_{2,} 3-membered carbocyclyl, 4-membered carbocyclyl, 5-membered carbocyclyl or 6-membered carbocyclyl; each R20 is independently and optionally substituted or unsubstituted with 1, 2, 3, 4, 5 or 6 substituents selected from -deuterium, —F, —Cl, —Br, methyl, ethyl, propyl, isopropyl, methoxy, ethoxy, propoxy, isopropoxy, oxo, —OH, —NH₂, —NHCH₃, —N(CH₃)₂, —CN, —C(═O) CH₃, —C(═O)OO, —OC(═O)O, —C(═O)NH₂, —C(═O)NH(CH₃), —C(═O)N(CH₃)₂, —NHC(═O)CH₃, —N(CH₃) Substituents of C(═O)CH₃, —S(O)₂NH₂, —S(O)₂NH(CH₃) or —S(O)₂N(CH₃)₂

In some embodiments, each R₃ and R₄ at each occurrence is independently selected from deuterium, hydrogen, halogen, —C₁₋₆ alkyl, —C₂₋₆ alkenyl, —C₂₋₆ alkynyl, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇ or —C₃₋₁₀ carbocyclyl, each heterocyclyl and heteroaryl at each occurrence independently contains 1, 2, 3 or 4 heteroatoms selected from N, O, S, S═O or S(═O)₂; each R₃ and R₄ at each occurrence is independently and optionally substituted or unsubstituted with 1, 2, 3, 4, 5 or 6 substituents selected from deuterium, halogen, oxo, —C₁₋₆ alkyl, —C₁₋₆ alkoxy, oxo, —OR₆, —NR₆R₇, —CN, —C(═O)R₆, —C(═O)OR₆, —OC(═O)R₆, Substituents of —C(═O)NR₆R₇, —NR₆C(═O)R₇ or —S(O)₂NR₆R₇.

In some embodiments, R₆ and R₇ in each of R₃ and R₄ at each occurrence are independently selected from hydrogen, deuterium, or —C₁₋₃ alkyl.

In some embodiments, each R₃ and R₄ at each occurrence is independently selected from hydrogen, deuterium, —F, —Cl, —Br, methyl, ethyl, propyl, isopropyl, vinyl, propylene base, isopropenyl, ethynyl, propynyl, oxo, —OH, —OCH₃, —OCH₂CH₃, —OCH₂CH₂CH₃, —OCH(CH₃)₂, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CN,—C(═O)CH₃, —C(═O)OCH₃, —OC(═O)CH₃, —C(═O)NH₂, —C(═O)NH(CH₃), —C(═O)N(CH₃)₂, —NHC(═O) CH₃, —N(CH₃)C(═O)CH₃, —S(O)₂NH₂, —S(O)₂NH(CH₃), —S(O)₂N(CH₃)₂, 3-membered carbocyclyl, 4-membered Carbocyclyl, 5-membered carbocyclyl, or 6-membered carbocyclyl; each R₃ and R₄ is independently and optionally substituted or unsubstituted with 1, 2, 3, 4, 5, or 6 substituents selected from deuterium, —F, —Cl, —Br, oxo, methyl, ethyl, propyl, isopropyl, —OH, OCH₃, —OCH₂CH₃, —OCH₂CH₂CH₃, —OCH(CH₃)₂, —NH₂, —N(CH₃)₂, —CN, —C(═O)CH₃, —OC(═O)CH₃, —C(═O)NH 2, —C(═O)NH(CH₃), —C(═O)N(CH₃)₂, —NHC(=Substituents of O)CH₃, —N(CH₃)C(═O)CH₃, —S(O)₂NH₂, —S(O)₂NH(CH₃), —S(O)₂N(CH₃)₂.

In some embodiments, each R₅ at each occurrence is independently selected from deuterium, —F, —Cl, —Br, —C₁₋₃ alkyl, —C₁₋₃ alkylene4halogen)₁₋₃, C₁₋₃ heteroalkyl, —C₂₋₃ alkenyl, —C₂₋₃ alkynyl, —CN, —OR₆, —C₁₋₆ alkylene —(OR₆)₁₋₃, —O—C₁₋₆ alkylene Alkyl-(halogen)₁₋₃, —SR₆, —S—C₁₋₆ alkylene-(halogen)₁₋₃, —NR₆R₇, —C₁₋₆ alkylene-NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl, each Heterocyclyl and heteroaryl at each occurrence independently contain 1, 2, 3 or 4 heteroatoms selected from N, O, S, S═O or S(═O)₂; each R₃ and R₄ at each occurrence independently and optionally substituted or unsubstituted with 1, 2, 3 or 4, 5 or 6 substituents selected from deuterium, —F, —Cl, —Br, oxo, —C₁₋₆ alkyl, —C₁₋₆ Alkoxy, oxo, —OR₆, —NR₆R₇, —CN, —C(═O)R₆, —C(═O)OR₆, —OC(═O)R₆, —C(═O)NR₆R₇, —NR₆C (═O) R₇ or —S(O)₂NR₆R₇.

In some embodiments, R₆ and R₇ in each R₅ at each occurrence are independently selected from hydrogen, deuterium or —C₁₋₃ alkyl, or R₆ and R₇ in R₅ together with the N atom to which they are commonly attached formed 3-6-membered heterocycles, the 3-6-membered heterocycle may further comprise 1 heteroatom selected from N, and the 3-6-membered heterocycle is independently and optionally replaced by 1, 2, 3, 4 heteroatom selected from N, O or S.

In some embodiments, each R₅ at each occurrence is independently selected from deuterium, —F, —Cl, —Br, methyl, ethyl, propyl, isopropyl, vinyl, propenyl, isopropyl propenyl, ethynyl, propynyl, -methylene-(halogen)₁₋₃, -ethylene-(halogen)₁₋₃, -propylene-(halogen)₁₋₃, heteromethyl, Heteroethyl, heteropropyl, vinyl, propenyl, ethynyl, propynyl, oxo, —OR₆, -methylene-(OR₆)₁₋₃, -ethylene-(OR₆)₁₋₃, -propylene-(OR₆)₁₋₃, —O-methylene-(halogen)₁₋₃, —O-ethylene-(halogen)₁₋₃, —O-propylene-(halogen))₁₋₃, —NR₆R₇,-methylene —NR₆R₇,-ethylene-NR₆R₇,-propylene-NR₆R₇, —CN,—C(═O)R₆, —C(═O)OR₆, —OC (═O)R₆, —C(═O)NR₆R₇, —NR₆C(═O)R₇, —S(O)₂NR₆R₇, phenyl, naphthyl, 5-membered heteroaryl, 6-membered heteroaryl, 7-membered heteroaryl Aryl, 6-membered heteroaryl, 8-membered heteroaryl, 10-membered heteroaryl, 3-membered heterocyclyl, 4-membered heterocyclyl, 5-membered heterocyclyl, 6-membered heterocyclyl, 3-membered carbocyclyl, 4-membered carbocyclyl, 5-membered carbocyclyl or 6-membered carbocyclyl, each heterocyclyl and heteroaryl at each occurrence independently contains 1, 2, 3 or 4 heteroatoms selected from N, O or S; each R₇ at each occurrence is independently and optionally replaced or not replaced by 1, 2, 3, 4, 5 or 6 substituents selected from —F, —Cl, —Br, oxo, methyl, ethyl, propyl, isopropyl, —OR₆, —NR₆R₇, —CN, —C(═O)R₆, —C(═O)OR₆, —OC(═O)R₆, —C(═O)NR₆R₇, NR₆C(═O)R₆, or —S(O)₂NR₆R₇.

In some embodiments, R₆ and R₇ in each R₅ are independently selected at each occurrence from hydrogen, deuterium, methyl, ethyl, propyl, isopropyl; or R₆ and R₇ in each R₅ together with he N atoms they are connected to form

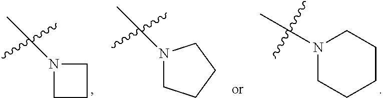 [SMILES: *N1CCC1 ; *N1CCCC1 ; *N1CCCCC1]

In some embodiments, each R₅ is independently selected at each occurrence from deuterium, —F, —Cl, —Br, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CH₂F₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —OCH₂F, —OCHF₂, —OCF₃, —OOOF, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SOF, —SCHF₂, —SCF₃, —SCH₂CH₂F, —SCH₂CH₂F₂, —SCH₂CF₃, —SCH₂CH₂CH₂F, —SCH₂CH₂CHF₂, —SCH₂CH₂CF₃, —NH₂, —NHCH₃, —NHCH₂CH₃, —NHCH₂CH₂CH₃, —NHCH(CH₃)₂, —N(CH₃)₂, —N(CH₃)CH₂CH₃, —N(O)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —NHC(═O)CH₃, —N(CH₃)C(═O) CH₃, —S(O)₂NH₂, —S(O)₂NH(CH₃), —S(O)₂N(CH₃)₂, 3-membered carbocyclyl, 4-membered carbocyclyl, 5-membered carbocyclyl, or 6-membered Carbocyclyl; each R₂₀ is independently and optionally substituted or unsubstituted with 1, 2, 3, 4, 5 or 6 substituents selected from -deuterium, —F, —Cl, —Br, methyl, ethyl, propyl, isopropyl base, methoxy, ethoxy, propoxy, isopropoxy, oxo, —OH, —NH₂, —NHCH₃, —N(CH₃)₂, —CN, —C(═O)CH₃, C(═O)OO, —OC(═O)O, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂.

In some embodiments, L₁-R₁₉ are selected from the following structures:

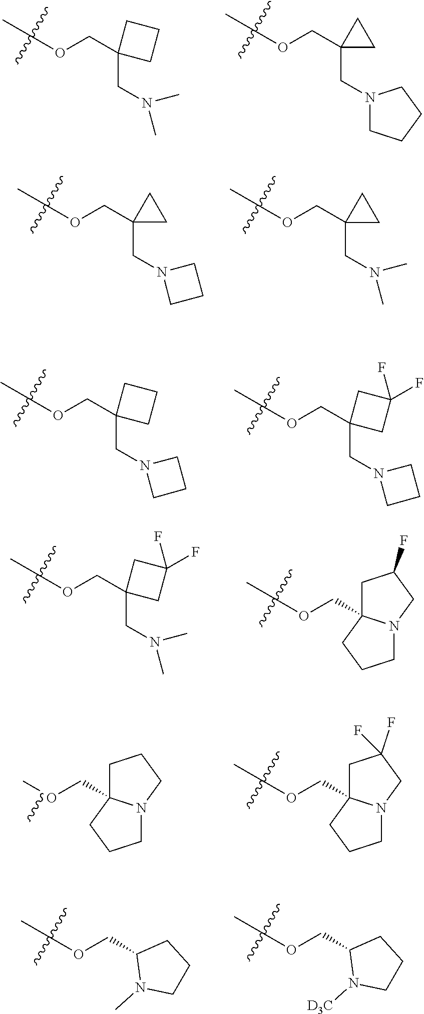

In some embodiments, the compound of formula (I) or an isomer, solvate or precursor thereof, or a pharmaceutically acceptable salt thereof is selected from the group consisting of the following compounds, isomers, solvates thereof or their precursors, or their pharmaceutically acceptable salts:

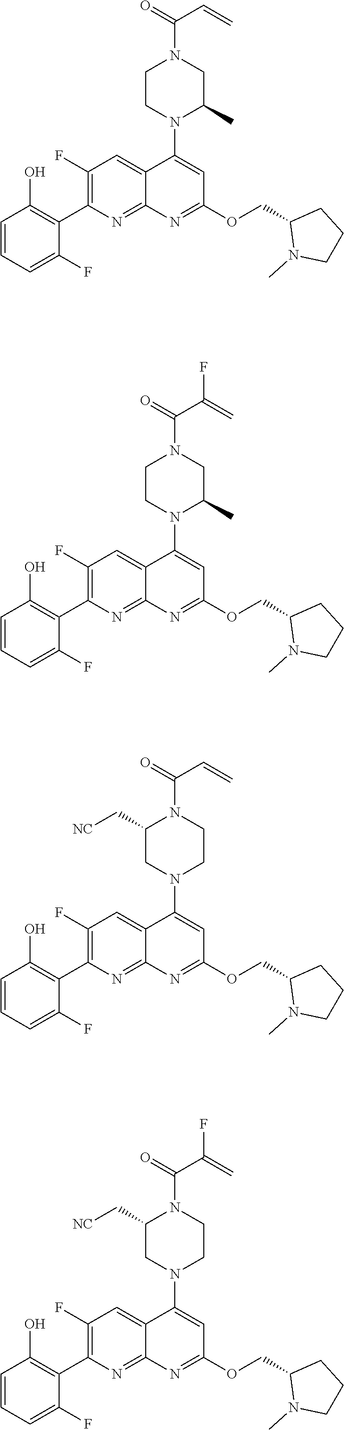 [SMILES: C=CC(=O)N1CCN(c2cc(OC[C@@H]3CCCN3C)nc3nc(-c4c(O)cccc4F)c(F)cc23)[C@H](C)C1 ; C=C(F)C(=O)N1CCN(c2cc(OC[C@@H]3CCCN3C)nc3nc(-c4c(O)cccc4F)c(F)cc23)[C@H](C)C1 ; C=CC(=O)N1CCN(c2cc(OC[C@@H]3CCCN3C)nc3nc(-c4c(O)cccc4F)c(F)cc23)C[C@@H]1CC#N ; C=C(F)C(=O)N1CCN(c2cc(OC[C@@H]3CCCN3C)nc3nc(-c4c(O)cccc4F)c(F)cc23)C[C@@H]1CC#N]

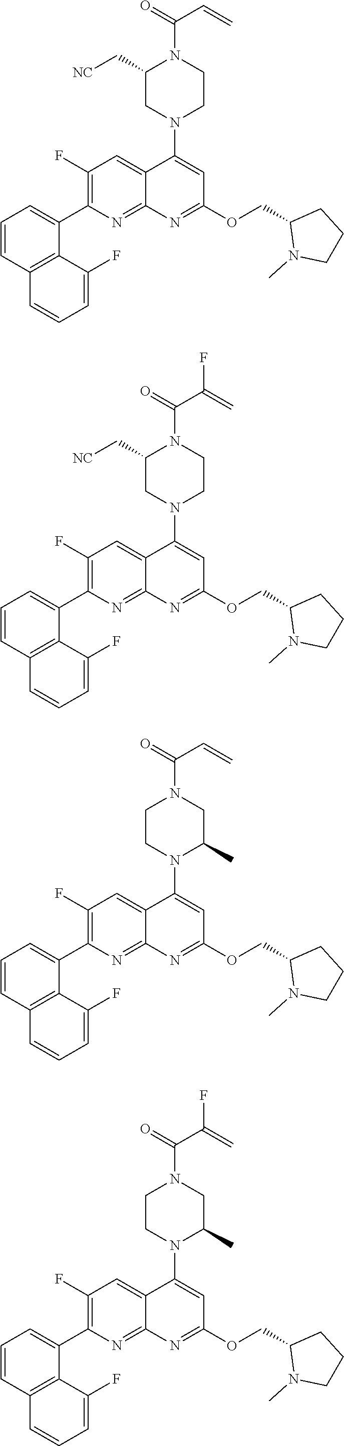 [SMILES: C=CC(=O)N1CCN(c2cc(OC[C@@H]3CCCN3C)nc3nc(-c4cccc5cccc(F)c45)c(F)cc23)C[C@@H]1CC#N ; C=C(F)C(=O)N1CCN(c2cc(OC[C@@H]3CCCN3C)nc3nc(-c4cccc5cccc(F)c45)c(F)cc23)C[C@@H]1CC#N ; C=CC(=O)N1CCN(c2cc(OC[C@@H]3CCCN3C)nc3nc(-c4cccc5cccc(F)c45)c(F)cc23)[C@H](C)C1 ; C=C(F)C(=O)N1CCN(c2cc(OC[C@@H]3CCCN3C)nc3nc(-c4cccc5cccc(F)c45)c(F)cc23)[C@H](C)C1]

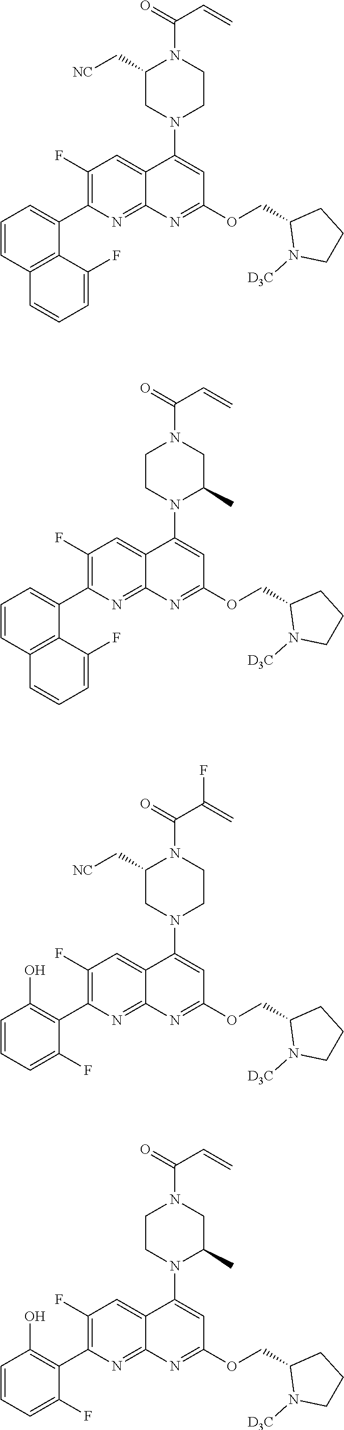 [SMILES: [2H]C([2H])([2H])N1CCC[C@H]1COc1cc(N2CCN(C(=O)C=C)[C@@H](CC#N)C2)c2cc(F)c(-c3cccc4cccc(F)c34)nc2n1 ; [2H]C([2H])([2H])N1CCC[C@H]1COc1cc(N2CCN(C(=O)C=C)C[C@H]2C)c2cc(F)c(-c3cccc4cccc(F)c34)nc2n1 ; [2H]C([2H])([2H])N1CCC[C@H]1COc1cc(N2CCN(C(=O)C(=C)F)[C@@H](CC#N)C2)c2cc(F)c(-c3c(O)cccc3F)nc2n1 ; [2H]C([2H])([2H])N1CCC[C@H]1COc1cc(N2CCN(C(=O)C=C)C[C@H]2C)c2cc(F)c(-c3c(O)cccc3F)nc2n1]

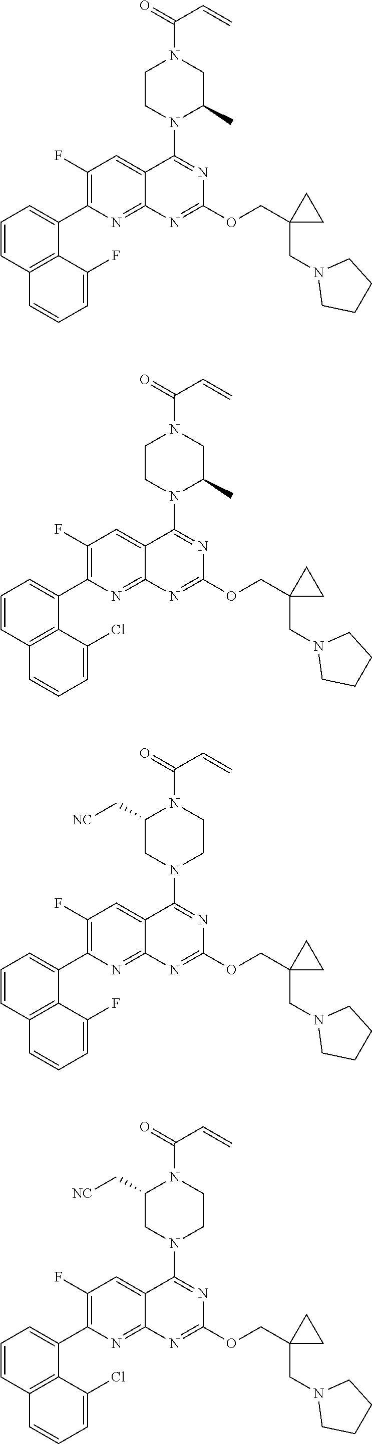 [SMILES: C=CC(=O)N1CCN(c2nc(OCC3(CN4CCCC4)CC3)nc3nc(-c4cccc5cccc(F)c45)c(F)cc23)[C@H](C)C1 ; C=CC(=O)N1CCN(c2nc(OCC3(CN4CCCC4)CC3)nc3nc(-c4cccc5cccc(Cl)c45)c(F)cc23)[C@H](C)C1 ; C=CC(=O)N1CCN(c2nc(OCC3(CN4CCCC4)CC3)nc3nc(-c4cccc5cccc(F)c45)c(F)cc23)C[C@@H]1CC#N ; C=CC(=O)N1CCN(c2nc(OCC3(CN4CCCC4)CC3)nc3nc(-c4cccc5cccc(Cl)c45)c(F)cc23)C[C@@H]1CC#N]

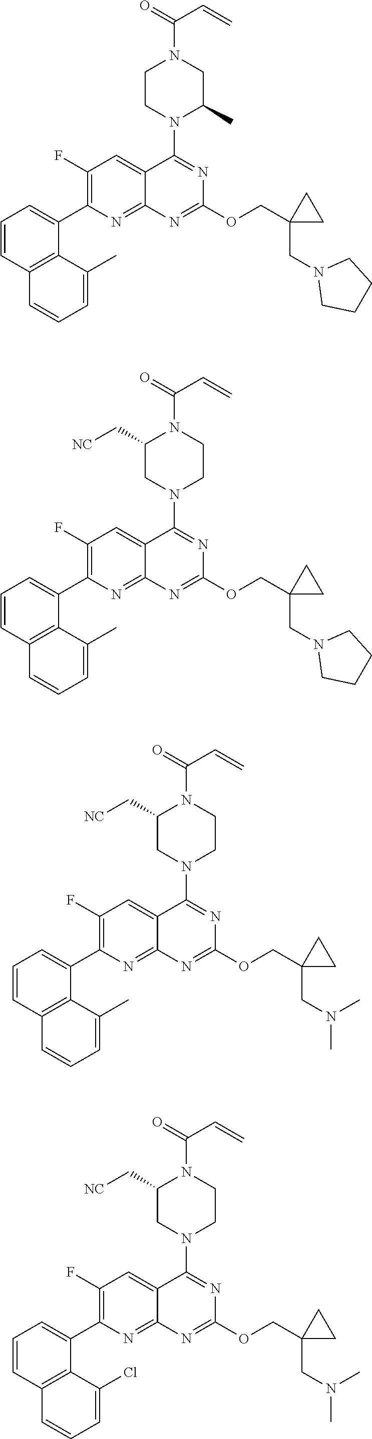 [SMILES: C=CC(=O)N1CCN(c2nc(OCC3(CN4CCCC4)CC3)nc3nc(-c4cccc5cccc(C)c45)c(F)cc23)[C@H](C)C1 ; C=CC(=O)N1CCN(c2nc(OCC3(CN4CCCC4)CC3)nc3nc(-c4cccc5cccc(C)c45)c(F)cc23)C[C@@H]1CC#N ; C=CC(=O)N1CCN(c2nc(OCC3(CN(C)C)CC3)nc3nc(-c4cccc5cccc(C)c45)c(F)cc23)C[C@@H]1CC#N ; C=CC(=O)N1CCN(c2nc(OCC3(CN(C)C)CC3)nc3nc(-c4cccc5cccc(Cl)c45)c(F)cc23)C[C@@H]1CC#N]

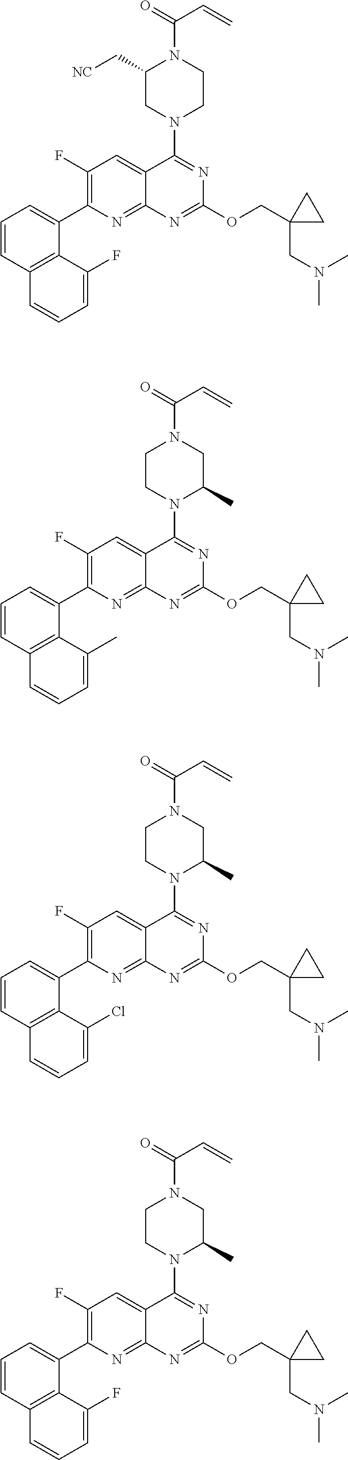 [SMILES: C=CC(=O)N1CCN(c2nc(OCC3(CN(C)C)CC3)nc3nc(-c4cccc5cccc(F)c45)c(F)cc23)C[C@@H]1CC#N ; C=CC(=O)N1CCN(c2nc(OCC3(CN(C)C)CC3)nc3nc(-c4cccc5cccc(C)c45)c(F)cc23)[C@H](C)C1 ; C=CC(=O)N1CCN(c2nc(OCC3(CN(C)C)CC3)nc3nc(-c4cccc5cccc(Cl)c45)c(F)cc23)[C@H](C)C1 ; C=CC(=O)N1CCN(c2nc(OCC3(CN(C)C)CC3)nc3nc(-c4cccc5cccc(F)c45)c(F)cc23)[C@H](C)C1]

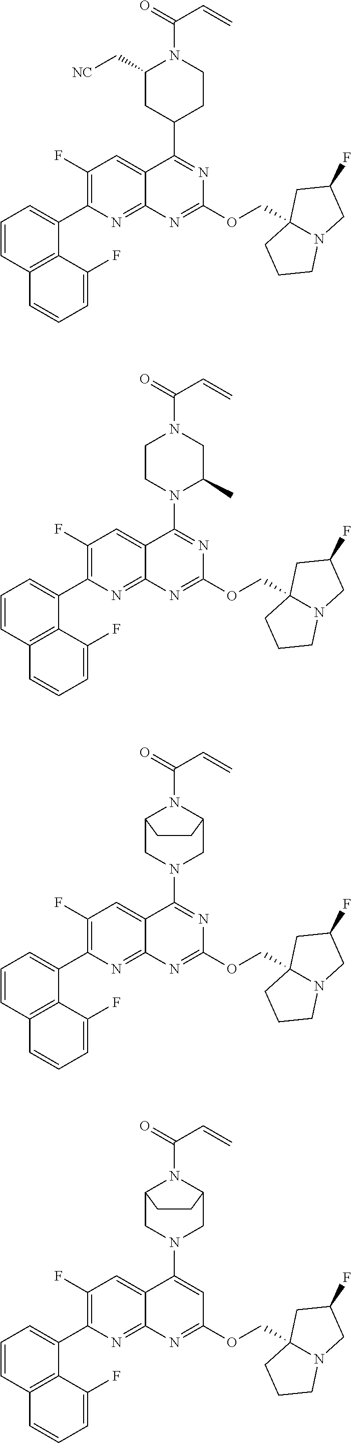 [SMILES: C=CC(=O)N1CCC(c2nc(OC[C@@]34CCCN3C[C@H](F)C4)nc3nc(-c4cccc5cccc(F)c45)c(F)cc23)C[C@@H]1CC#N ; C=CC(=O)N1CCN(c2nc(OC[C@@]34CCCN3C[C@H](F)C4)nc3nc(-c4cccc5cccc(F)c45)c(F)cc23)[C@H](C)C1 ; C=CC(=O)N1C2CCC1CN(c1nc(OC[C@@]34CCCN3C[C@H](F)C4)nc3nc(-c4cccc5cccc(F)c45)c(F)cc13)C2 ; C=CC(=O)N1C2CCC1CN(c1cc(OC[C@@]34CCCN3C[C@H](F)C4)nc3nc(-c4cccc5cccc(F)c45)c(F)cc13)C2]

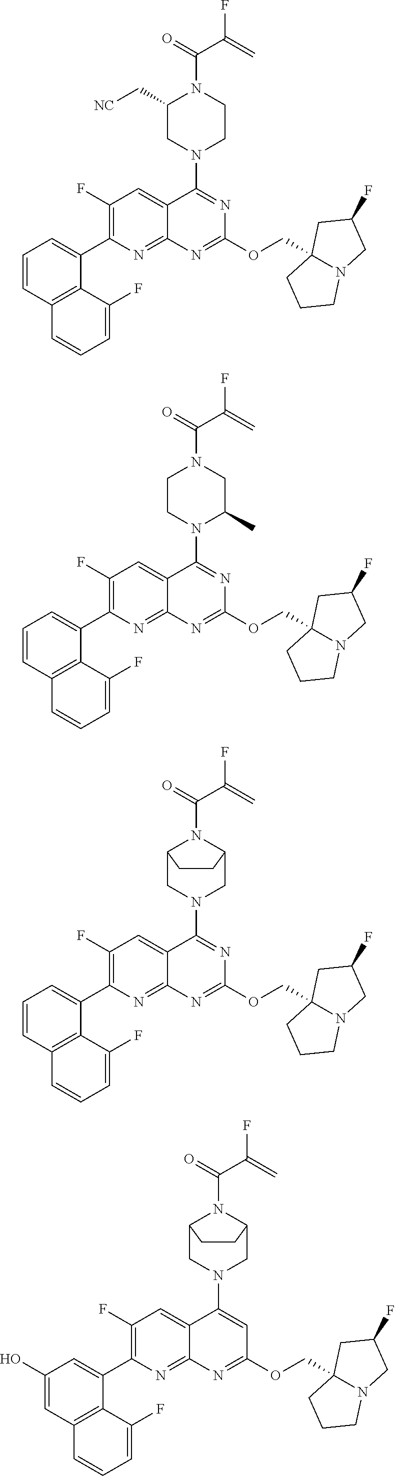 [SMILES: C=C(F)C(=O)N1CCN(c2nc(OC[C@@]34CCCN3C[C@H](F)C4)nc3nc(-c4cccc5cccc(F)c45)c(F)cc23)C[C@@H]1CC#N ; C=C(F)C(=O)N1CCN(c2nc(OC[C@@]34CCCN3C[C@H](F)C4)nc3nc(-c4cccc5cccc(F)c45)c(F)cc23)[C@H](C)C1 ; C=C(F)C(=O)N1C2CCC1CN(c1nc(OC[C@@]34CCCN3C[C@H](F)C4)nc3nc(-c4cccc5cccc(F)c45)c(F)cc13)C2 ; C=C(F)C(=O)N1C2CCC1CN(c1cc(OC[C@@]34CCCN3C[C@H](F)C4)nc3nc(-c4cc(O)cc5cccc(F)c45)c(F)cc13)C2]

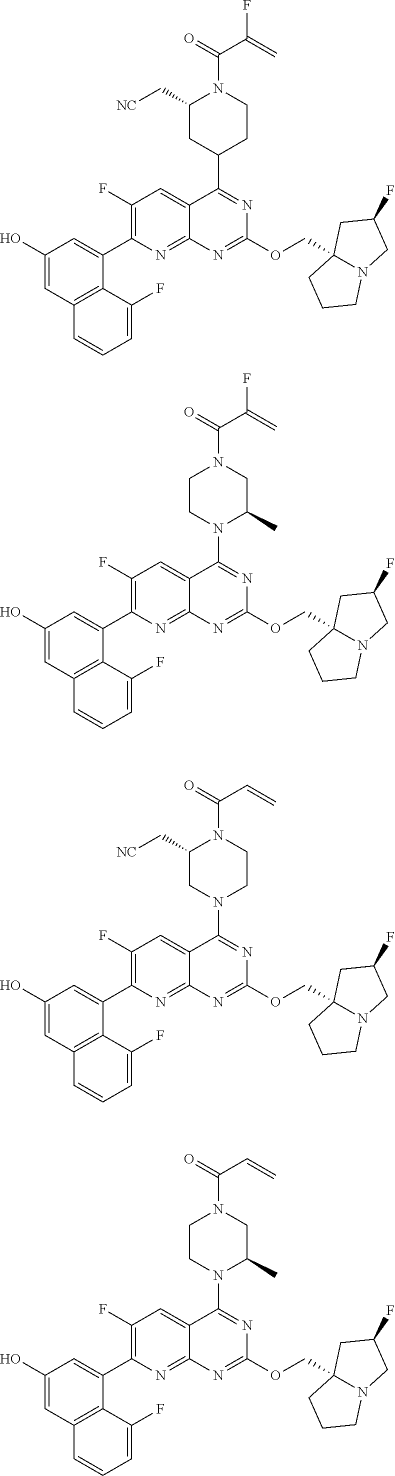 [SMILES: C=C(F)C(=O)N1CCC(c2nc(OC[C@@]34CCCN3C[C@H](F)C4)nc3nc(-c4cc(O)cc5cccc(F)c45)c(F)cc23)C[C@@H]1CC#N ; C=C(F)C(=O)N1CCN(c2nc(OC[C@@]34CCCN3C[C@H](F)C4)nc3nc(-c4cc(O)cc5cccc(F)c45)c(F)cc23)[C@H](C)C1 ; C=CC(=O)N1CCN(c2nc(OC[C@@]34CCCN3C[C@H](F)C4)nc3nc(-c4cc(O)cc5cccc(F)c45)c(F)cc23)C[C@@H]1CC#N ; C=CC(=O)N1CCN(c2nc(OC[C@@]34CCCN3C[C@H](F)C4)nc3nc(-c4cc(O)cc5cccc(F)c45)c(F)cc23)[C@H](C)C1]

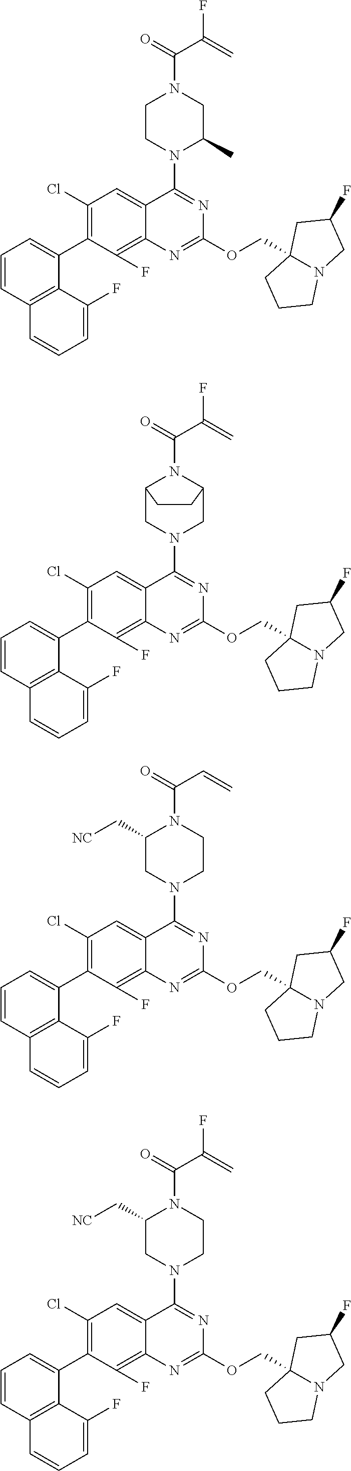 [SMILES: C=C(F)C(=O)N1CCN(c2nc(OC[C@@]34CCCN3C[C@H](F)C4)nc3c(F)c(-c4cccc5cccc(F)c45)c(Cl)cc23)[C@H](C)C1 ; C=C(F)C(=O)N1C2CCC1CN(c1nc(OC[C@@]34CCCN3C[C@H](F)C4)nc3c(F)c(-c4cccc5cccc(F)c45)c(Cl)cc13)C2 ; C=CC(=O)N1CCN(c2nc(OC[C@@]34CCCN3C[C@H](F)C4)nc3c(F)c(-c4cccc5cccc(F)c45)c(Cl)cc23)C[C@@H]1CC#N ; C=C(F)C(=O)N1CCN(c2nc(OC[C@@]34CCCN3C[C@H](F)C4)nc3c(F)c(-c4cccc5cccc(F)c45)c(Cl)cc23)C[C@@H]1CC#N]

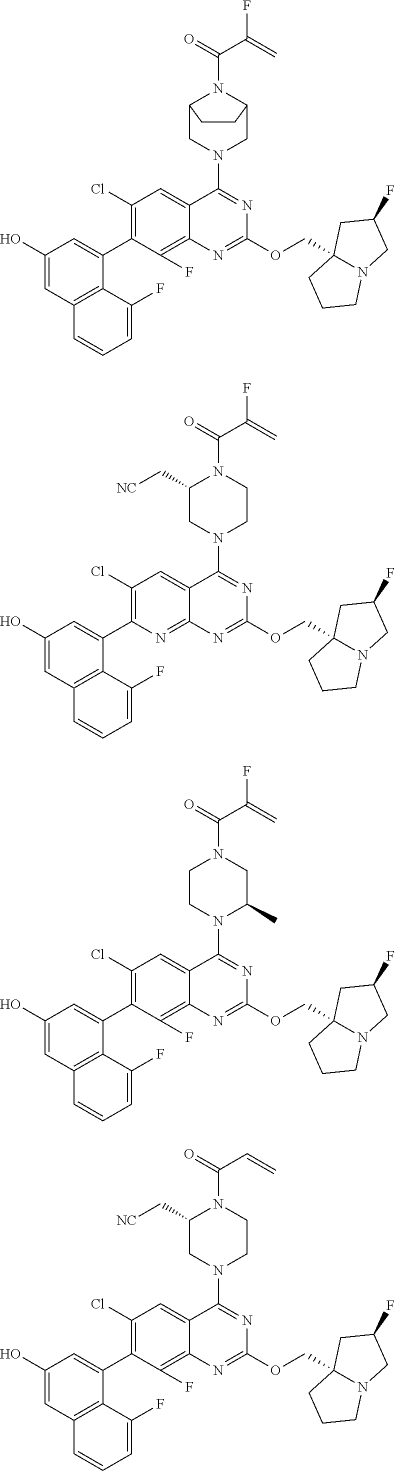 [SMILES: C=C(F)C(=O)N1C2CCC1CN(c1nc(OC[C@@]34CCCN3C[C@H](F)C4)nc3c(F)c(-c4cc(O)cc5cccc(F)c45)c(Cl)cc13)C2 ; C=C(F)C(=O)N1CCN(c2nc(OC[C@@]34CCCN3C[C@H](F)C4)nc3nc(-c4cc(O)cc5cccc(F)c45)c(Cl)cc23)C[C@@H]1CC#N ; C=C(F)C(=O)N1CCN(c2nc(OC[C@@]34CCCN3C[C@H](F)C4)nc3c(F)c(-c4cc(O)cc5cccc(F)c45)c(Cl)cc23)[C@H](C)C1 ; C=CC(=O)N1CCN(c2nc(OC[C@@]34CCCN3C[C@H](F)C4)nc3c(F)c(-c4cc(O)cc5cccc(F)c45)c(Cl)cc23)C[C@@H]1CC#N]

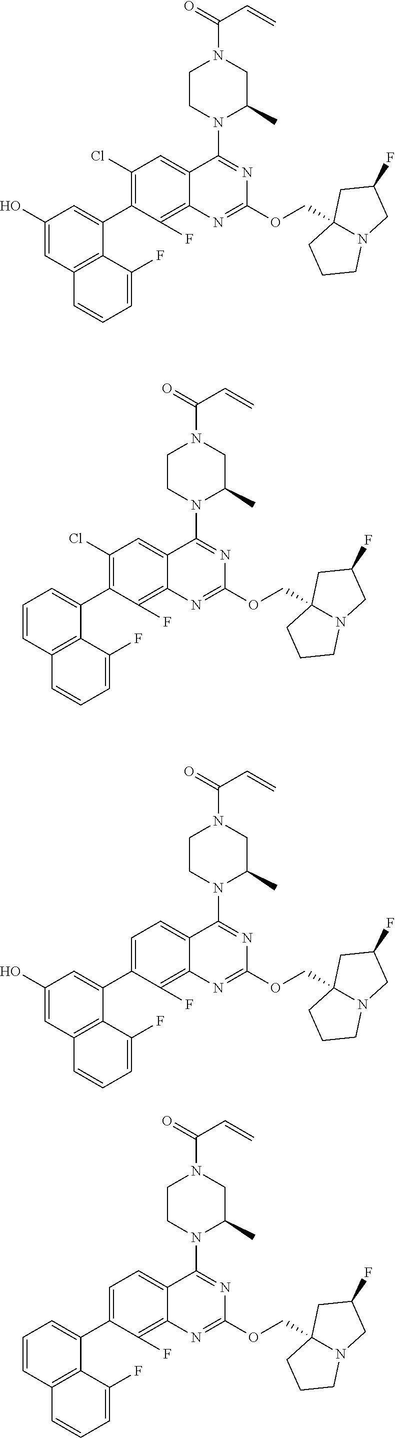 [SMILES: C=CC(=O)N1CCN(c2nc(OC[C@@]34CCCN3C[C@H](F)C4)nc3c(F)c(-c4cc(O)cc5cccc(F)c45)c(Cl)cc23)[C@H](C)C1 ; C=CC(=O)N1CCN(c2nc(OC[C@@]34CCCN3C[C@H](F)C4)nc3c(F)c(-c4cccc5cccc(F)c45)c(Cl)cc23)[C@H](C)C1 ; C=CC(=O)N1CCN(c2nc(OC[C@@]34CCCN3C[C@H](F)C4)nc3c(F)c(-c4cc(O)cc5cccc(F)c45)ccc23)[C@H](C)C1 ; C=CC(=O)N1CCN(c2nc(OC[C@@]34CCCN3C[C@H](F)C4)nc3c(F)c(-c4cccc5cccc(F)c45)ccc23)[C@H](C)C1]

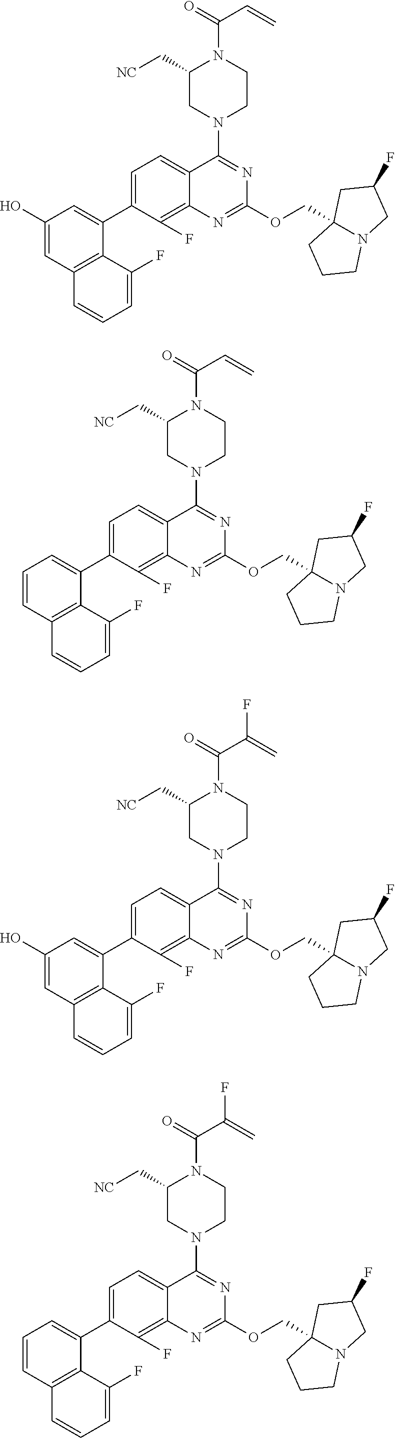 [SMILES: C=CC(=O)N1CCN(c2nc(OC[C@@]34CCCN3C[C@H](F)C4)nc3c(F)c(-c4cc(O)cc5cccc(F)c45)ccc23)C[C@@H]1CC#N ; C=CC(=O)N1CCN(c2nc(OC[C@@]34CCCN3C[C@H](F)C4)nc3c(F)c(-c4cccc5cccc(F)c45)ccc23)C[C@@H]1CC#N ; C=C(F)C(=O)N1CCN(c2nc(OC[C@@]34CCCN3C[C@H](F)C4)nc3c(F)c(-c4cc(O)cc5cccc(F)c45)ccc23)C[C@@H]1CC#N ; C=C(F)C(=O)N1CCN(c2nc(OC[C@@]34CCCN3C[C@H](F)C4)nc3c(F)c(-c4cccc5cccc(F)c45)ccc23)C[C@@H]1CC#N]

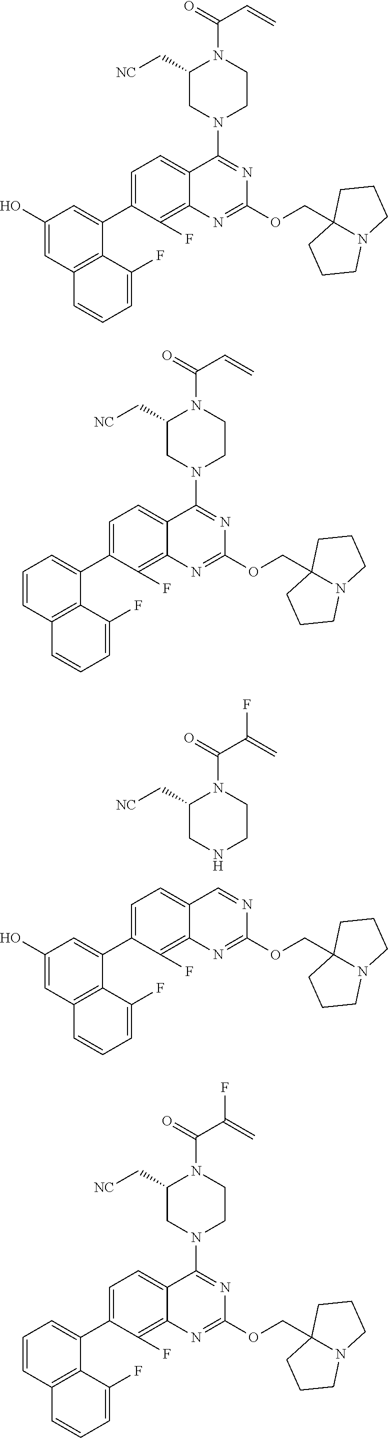

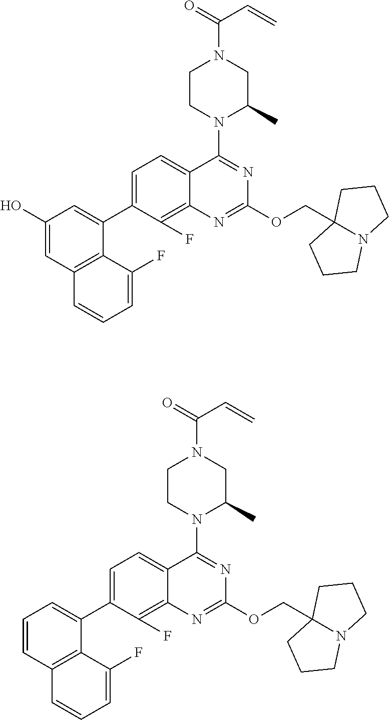 [SMILES: C=CC(=O)N1CCN(c2nc(OCC34CCCN3CCC4)nc3c(F)c(-c4cc(O)cc5cccc(F)c45)ccc23)[C@H](C)C1 ; C=CC(=O)N1CCN(c2nc(OCC34CCCN3CCC4)nc3c(F)c(-c4cccc5cccc(F)c45)ccc23)[C@H](C)C1]

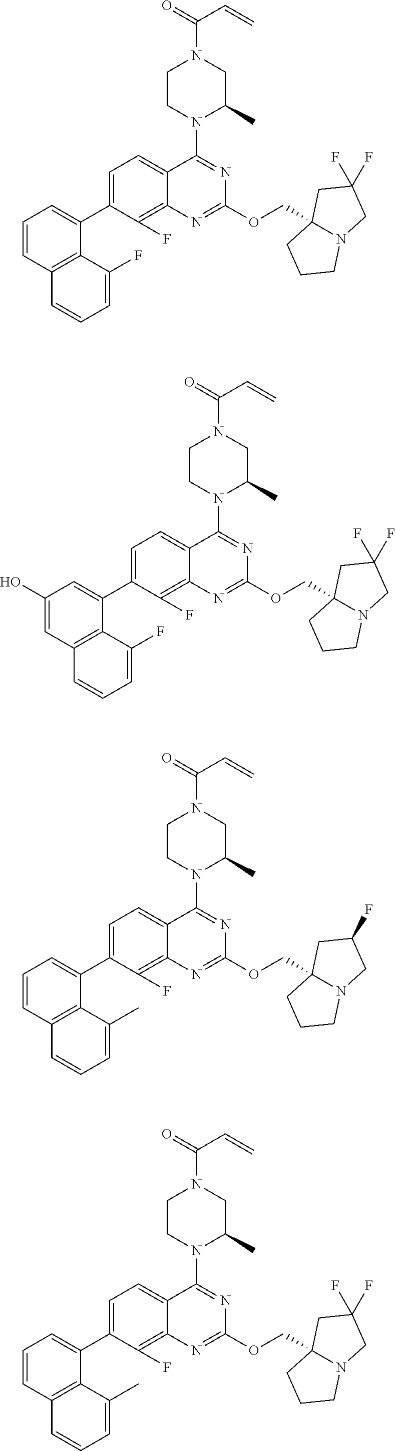 [SMILES: C=CC(=O)N1CCN(c2nc(OC[C@@]34CCCN3CC(F)(F)C4)nc3c(F)c(-c4cccc5cccc(F)c45)ccc23)[C@H](C)C1 ; C=CC(=O)N1CCN(c2nc(OC[C@@]34CCCN3CC(F)(F)C4)nc3c(F)c(-c4cc(O)cc5cccc(F)c45)ccc23)[C@H](C)C1 ; C=CC(=O)N1CCN(c2nc(OC[C@@]34CCCN3C[C@H](F)C4)nc3c(F)c(-c4cccc5cccc(C)c45)ccc23)[C@H](C)C1 ; C=CC(=O)N1CCN(c2nc(OC[C@@]34CCCN3CC(F)(F)C4)nc3c(F)c(-c4cccc5cccc(C)c45)ccc23)[C@H](C)C1]

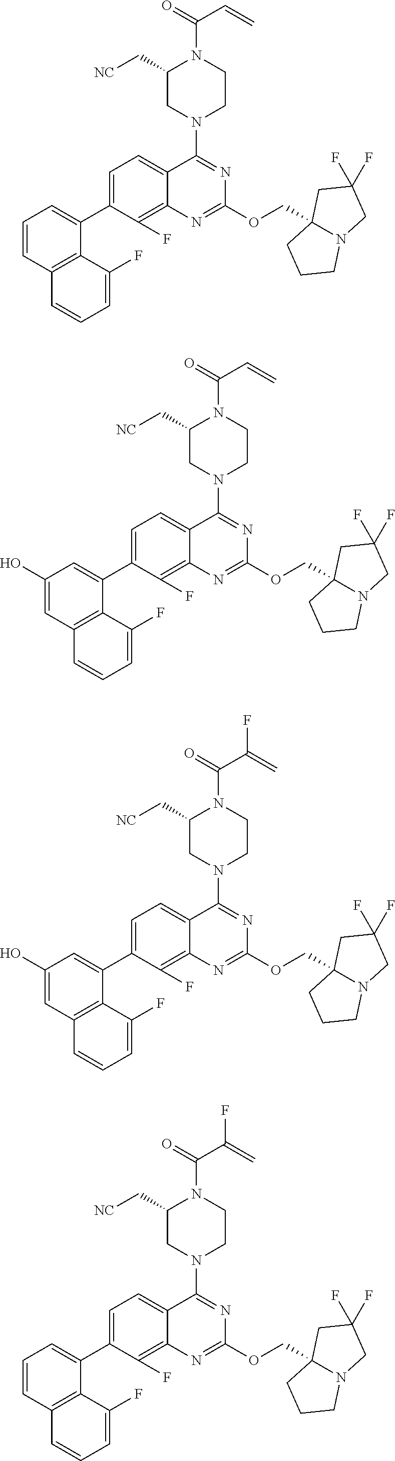 [SMILES: C=CC(=O)N1CCN(c2nc(OC[C@@]34CCCN3CC(F)(F)C4)nc3c(F)c(-c4cccc5cccc(F)c45)ccc23)C[C@@H]1CC#N ; C=CC(=O)N1CCN(c2nc(OC[C@@]34CCCN3CC(F)(F)C4)nc3c(F)c(-c4cc(O)cc5cccc(F)c45)ccc23)C[C@@H]1CC#N ; C=C(F)C(=O)N1CCN(c2nc(OC[C@@]34CCCN3CC(F)(F)C4)nc3c(F)c(-c4cc(O)cc5cccc(F)c45)ccc23)C[C@@H]1CC#N ; C=C(F)C(=O)N1CCN(c2nc(OC[C@@]34CCCN3CC(F)(F)C4)nc3c(F)c(-c4cccc5cccc(F)c45)ccc23)C[C@@H]1CC#N]

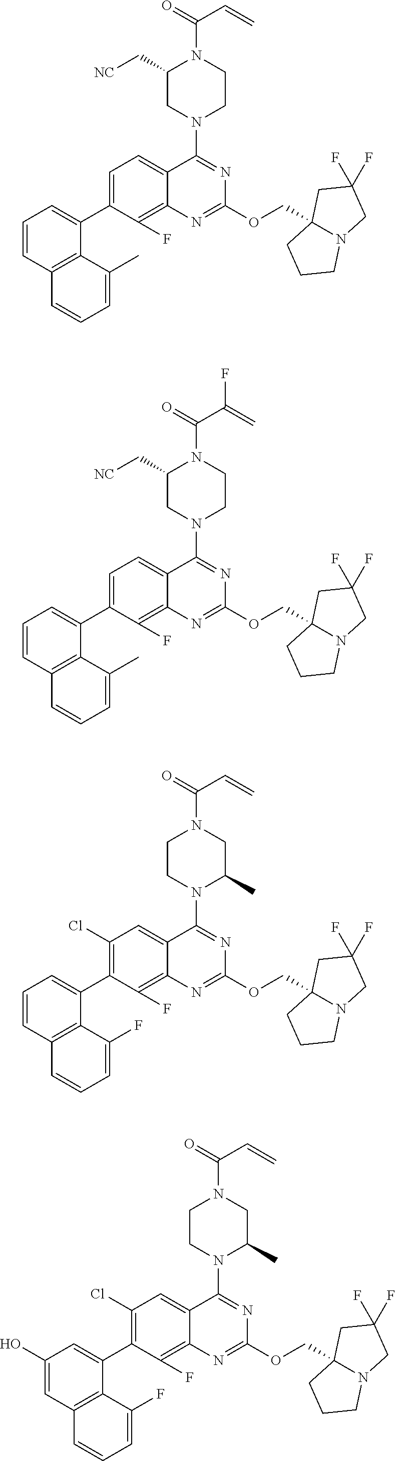 [SMILES: C=CC(=O)N1CCN(c2nc(OC[C@@]34CCCN3CC(F)(F)C4)nc3c(F)c(-c4cccc5cccc(C)c45)ccc23)C[C@@H]1CC#N ; C=C(F)C(=O)N1CCN(c2nc(OC[C@@]34CCCN3CC(F)(F)C4)nc3c(F)c(-c4cccc5cccc(C)c45)ccc23)C[C@@H]1CC#N ; C=CC(=O)N1CCN(c2nc(OC[C@@]34CCCN3CC(F)(F)C4)nc3c(F)c(-c4cccc5cccc(F)c45)c(Cl)cc23)[C@H](C)C1 ; C=CC(=O)N1CCN(c2nc(OC[C@@]34CCCN3CC(F)(F)C4)nc3c(F)c(-c4cc(O)cc5cccc(F)c45)c(Cl)cc23)[C@H](C)C1]

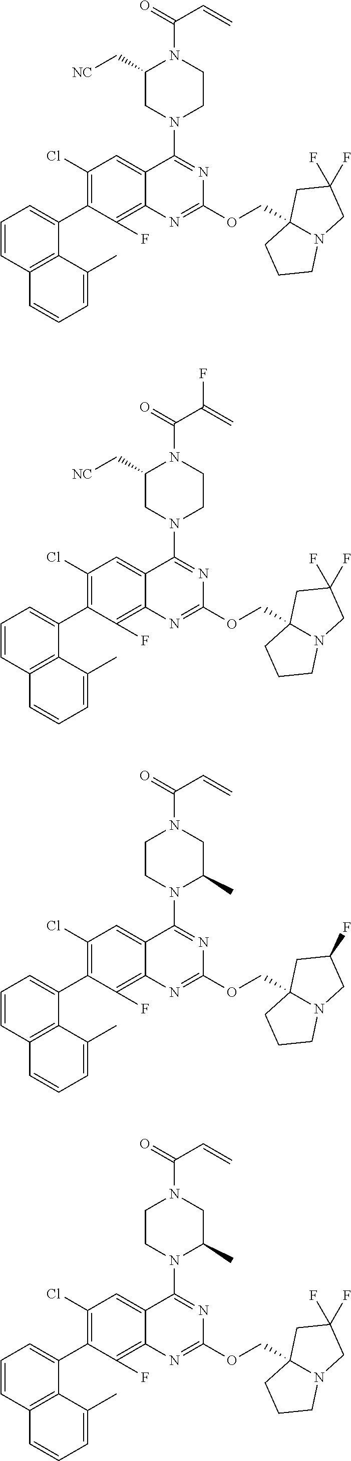 [SMILES: C=CC(=O)N1CCN(c2nc(OC[C@@]34CCCN3CC(F)(F)C4)nc3c(F)c(-c4cccc5cccc(C)c45)c(Cl)cc23)C[C@@H]1CC#N ; C=C(F)C(=O)N1CCN(c2nc(OC[C@@]34CCCN3CC(F)(F)C4)nc3c(F)c(-c4cccc5cccc(C)c45)c(Cl)cc23)C[C@@H]1CC#N ; C=CC(=O)N1CCN(c2nc(OC[C@@]34CCCN3C[C@H](F)C4)nc3c(F)c(-c4cccc5cccc(C)c45)c(Cl)cc23)[C@H](C)C1 ; C=CC(=O)N1CCN(c2nc(OC[C@@]34CCCN3CC(F)(F)C4)nc3c(F)c(-c4cccc5cccc(C)c45)c(Cl)cc23)[C@H](C)C1]

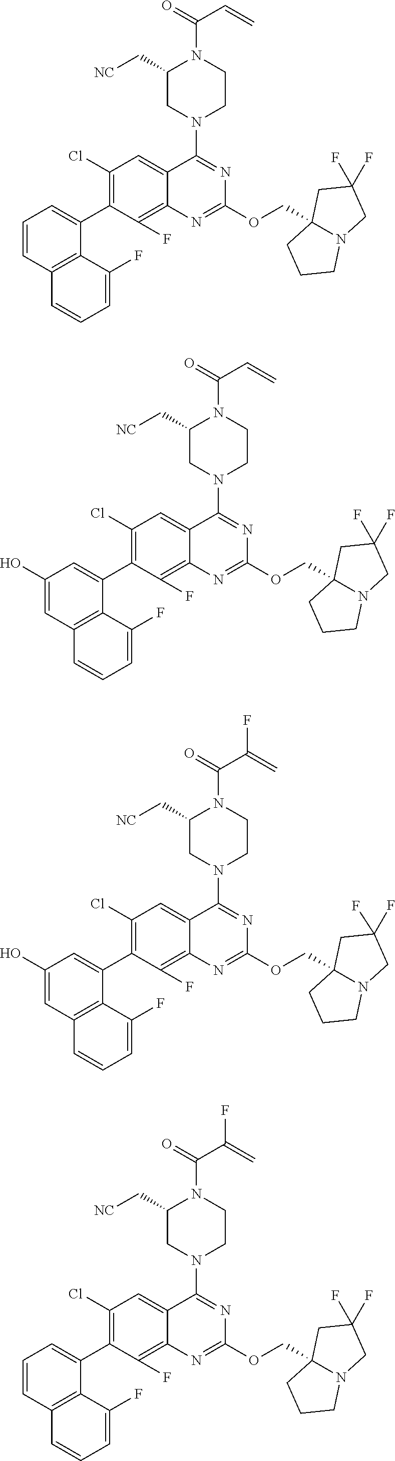 [SMILES: C=CC(=O)N1CCN(c2nc(OC[C@@]34CCCN3CC(F)(F)C4)nc3c(F)c(-c4cccc5cccc(F)c45)c(Cl)cc23)C[C@@H]1CC#N ; C=CC(=O)N1CCN(c2nc(OC[C@@]34CCCN3CC(F)(F)C4)nc3c(F)c(-c4cc(O)cc5cccc(F)c45)c(Cl)cc23)C[C@@H]1CC#N ; C=C(F)C(=O)N1CCN(c2nc(OC[C@@]34CCCN3CC(F)(F)C4)nc3c(F)c(-c4cc(O)cc5cccc(F)c45)c(Cl)cc23)C[C@@H]1CC#N ; C=C(F)C(=O)N1CCN(c2nc(OC[C@@]34CCCN3CC(F)(F)C4)nc3c(F)c(-c4cccc5cccc(F)c45)c(Cl)cc23)C[C@@H]1CC#N]

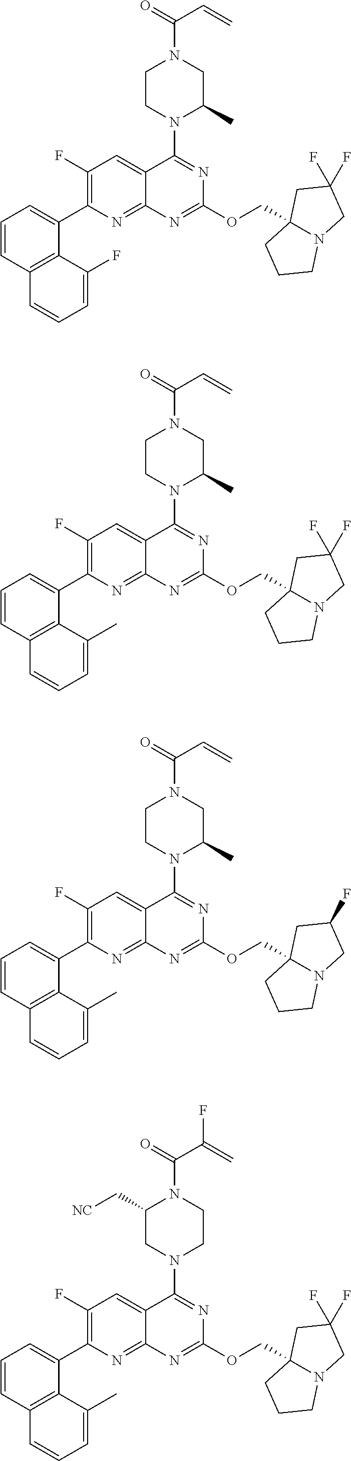 [SMILES: C=CC(=O)N1CCN(c2nc(OC[C@@]34CCCN3CC(F)(F)C4)nc3nc(-c4cccc5cccc(F)c45)c(F)cc23)[C@H](C)C1 ; C=CC(=O)N1CCN(c2nc(OC[C@@]34CCCN3CC(F)(F)C4)nc3nc(-c4cccc5cccc(C)c45)c(F)cc23)[C@H](C)C1 ; C=CC(=O)N1CCN(c2nc(OC[C@@]34CCCN3C[C@H](F)C4)nc3nc(-c4cccc5cccc(C)c45)c(F)cc23)[C@H](C)C1 ; C=C(F)C(=O)N1CCN(c2nc(OC[C@@]34CCCN3CC(F)(F)C4)nc3nc(-c4cccc5cccc(C)c45)c(F)cc23)C[C@@H]1CC#N]

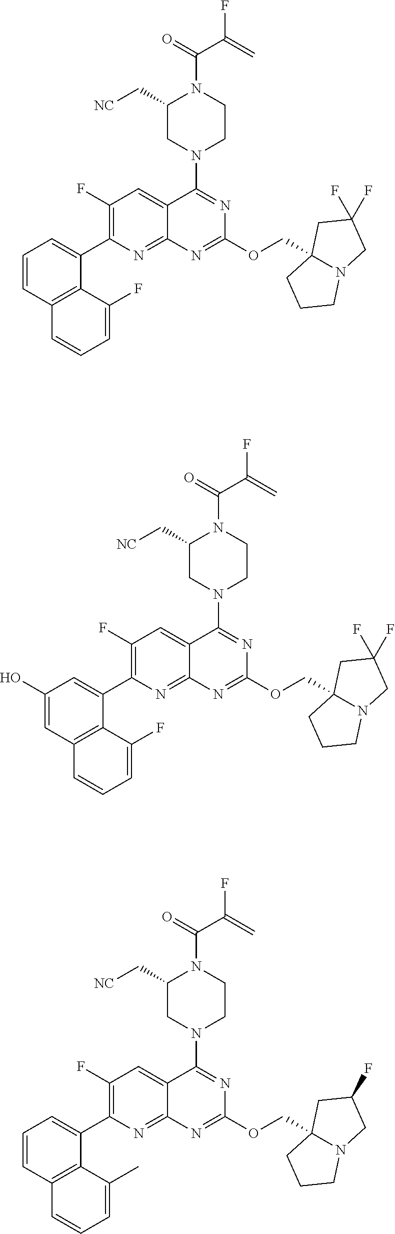 [SMILES: C=C(F)C(=O)N1CCN(c2nc(OC[C@@]34CCCN3CC(F)(F)C4)nc3nc(-c4cccc5cccc(F)c45)c(F)cc23)C[C@@H]1CC#N ; C=C(F)C(=O)N1CCN(c2nc(OC[C@@]34CCCN3CC(F)(F)C4)nc3nc(-c4cc(O)cc5cccc(F)c45)c(F)cc23)C[C@@H]1CC#N ; C=C(F)C(=O)N1CCN(c2nc(OC[C@@]34CCCN3C[C@H](F)C4)nc3nc(-c4cccc5cccc(C)c45)c(F)cc23)C[C@@H]1CC#N]

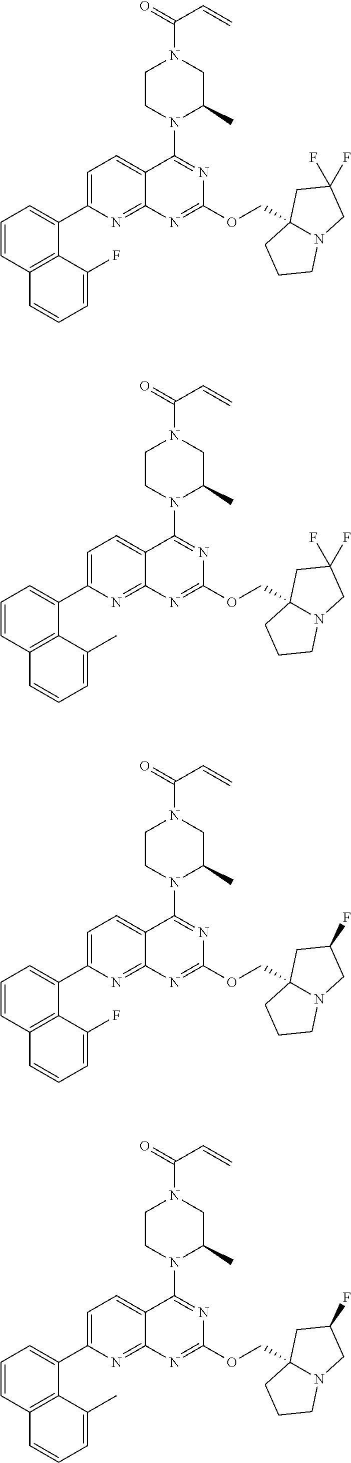 [SMILES: C=CC(=O)N1CCN(c2nc(OC[C@@]34CCCN3CC(F)(F)C4)nc3nc(-c4cccc5cccc(F)c45)ccc23)[C@H](C)C1 ; C=CC(=O)N1CCN(c2nc(OC[C@@]34CCCN3CC(F)(F)C4)nc3nc(-c4cccc5cccc(C)c45)ccc23)[C@H](C)C1 ; C=CC(=O)N1CCN(c2nc(OC[C@@]34CCCN3C[C@H](F)C4)nc3nc(-c4cccc5cccc(F)c45)ccc23)[C@H](C)C1 ; C=CC(=O)N1CCN(c2nc(OC[C@@]34CCCN3C[C@H](F)C4)nc3nc(-c4cccc5cccc(C)c45)ccc23)[C@H](C)C1]

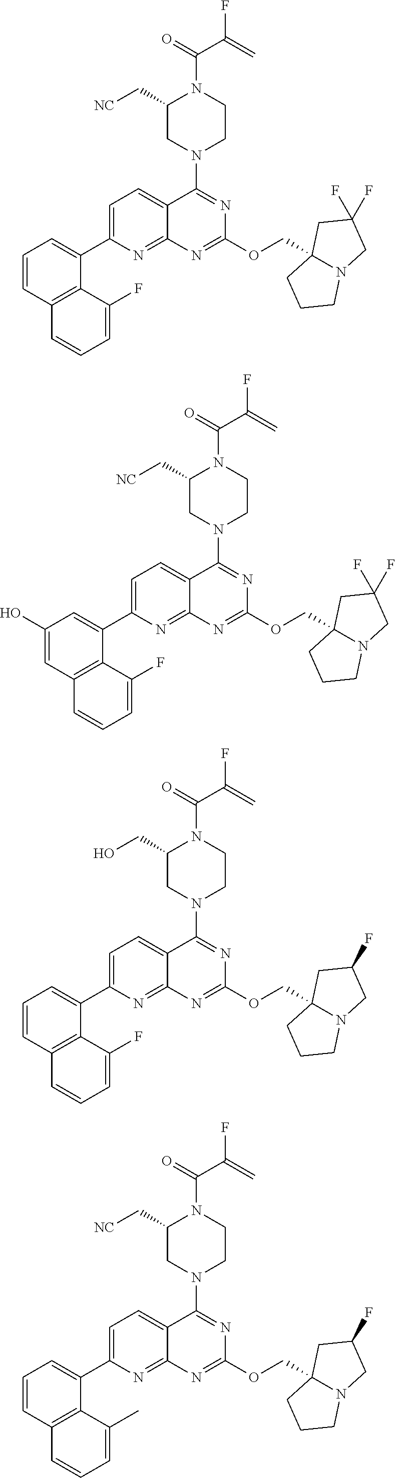 [SMILES: C=C(F)C(=O)N1CCN(c2nc(OC[C@@]34CCCN3CC(F)(F)C4)nc3nc(-c4cccc5cccc(F)c45)ccc23)C[C@@H]1CC#N ; C=C(F)C(=O)N1CCN(c2nc(OC[C@@]34CCCN3CC(F)(F)C4)nc3nc(-c4cc(O)cc5cccc(F)c45)ccc23)C[C@@H]1CC#N ; C=C(F)C(=O)N1CCN(c2nc(OC[C@@]34CCCN3C[C@H](F)C4)nc3nc(-c4cccc5cccc(F)c45)ccc23)C[C@@H]1CO ; C=C(F)C(=O)N1CCN(c2nc(OC[C@@]34CCCN3C[C@H](F)C4)nc3nc(-c4cccc5cccc(C)c45)ccc23)C[C@@H]1CC#N]

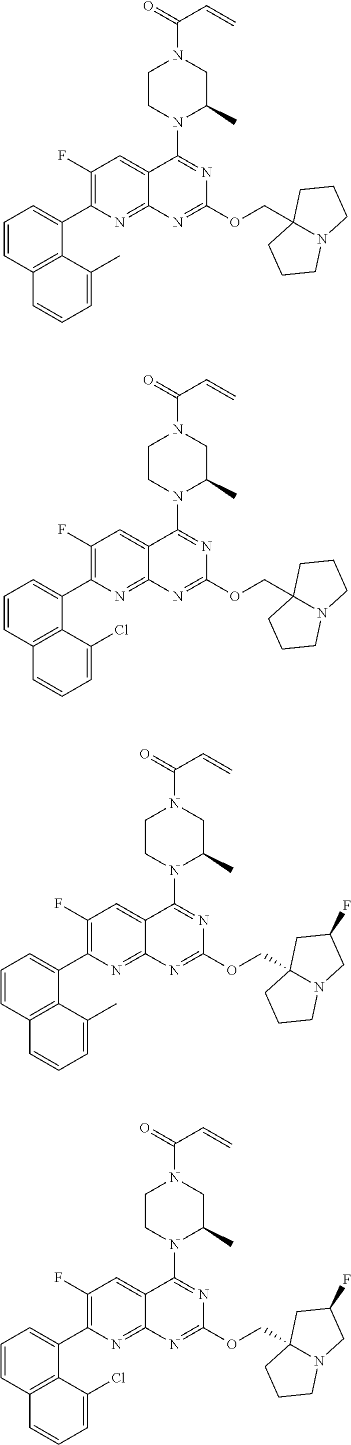 [SMILES: C=CC(=O)N1CCN(c2nc(OCC34CCCN3CCC4)nc3nc(-c4cccc5cccc(C)c45)c(F)cc23)[C@H](C)C1 ; C=CC(=O)N1CCN(c2nc(OCC34CCCN3CCC4)nc3nc(-c4cccc5cccc(Cl)c45)c(F)cc23)[C@H](C)C1 ; C=CC(=O)N1CCN(c2nc(OC[C@@]34CCCN3C[C@H](F)C4)nc3nc(-c4cccc5cccc(C)c45)c(F)cc23)[C@H](C)C1 ; C=CC(=O)N1CCN(c2nc(OC[C@@]34CCCN3C[C@H](F)C4)nc3nc(-c4cccc5cccc(Cl)c45)c(F)cc23)[C@H](C)C1]

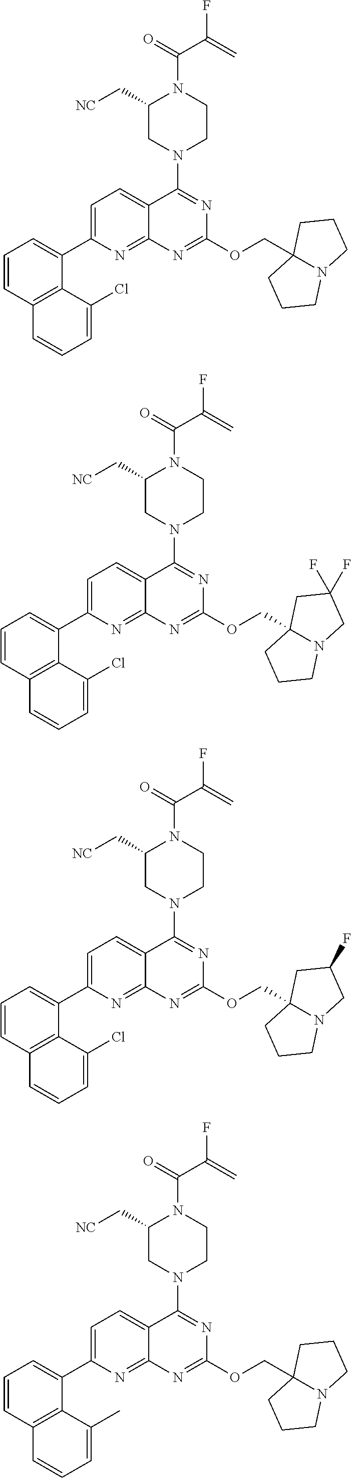 [SMILES: C=C(F)C(=O)N1CCN(c2nc(OCC34CCCN3CCC4)nc3nc(-c4cccc5cccc(Cl)c45)ccc23)C[C@@H]1CC#N ; C=C(F)C(=O)N1CCN(c2nc(OC[C@@]34CCCN3CC(F)(F)C4)nc3nc(-c4cccc5cccc(Cl)c45)ccc23)C[C@@H]1CC#N ; C=C(F)C(=O)N1CCN(c2nc(OC[C@@]34CCCN3C[C@H](F)C4)nc3nc(-c4cccc5cccc(Cl)c45)ccc23)C[C@@H]1CC#N ; C=C(F)C(=O)N1CCN(c2nc(OCC34CCCN3CCC4)nc3nc(-c4cccc5cccc(C)c45)ccc23)C[C@@H]1CC#N]

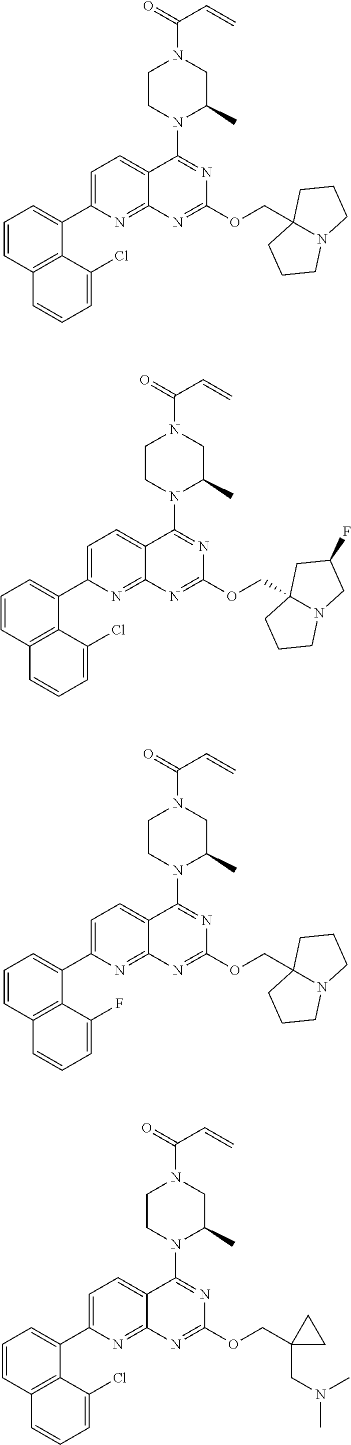 [SMILES: C=CC(=O)N1CCN(c2nc(OCC34CCCN3CCC4)nc3nc(-c4cccc5cccc(Cl)c45)ccc23)[C@H](C)C1 ; C=CC(=O)N1CCN(c2nc(OC[C@@]34CCCN3C[C@H](F)C4)nc3nc(-c4cccc5cccc(Cl)c45)ccc23)[C@H](C)C1 ; C=CC(=O)N1CCN(c2nc(OCC34CCCN3CCC4)nc3nc(-c4cccc5cccc(F)c45)ccc23)[C@H](C)C1 ; C=CC(=O)N1CCN(c2nc(OCC3(CN(C)C)CC3)nc3nc(-c4cccc5cccc(Cl)c45)ccc23)[C@H](C)C1]

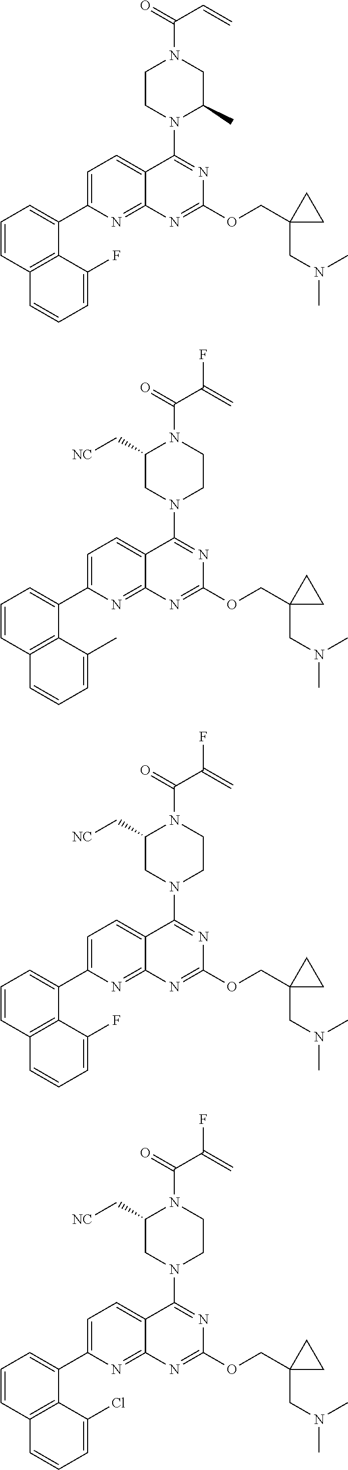 [SMILES: C=CC(=O)N1CCN(c2nc(OCC3(CN(C)C)CC3)nc3nc(-c4cccc5cccc(F)c45)ccc23)[C@H](C)C1 ; C=C(F)C(=O)N1CCN(c2nc(OCC3(CN(C)C)CC3)nc3nc(-c4cccc5cccc(C)c45)ccc23)C[C@@H]1CC#N ; C=C(F)C(=O)N1CCN(c2nc(OCC3(CN(C)C)CC3)nc3nc(-c4cccc5cccc(F)c45)ccc23)C[C@@H]1CC#N ; C=C(F)C(=O)N1CCN(c2nc(OCC3(CN(C)C)CC3)nc3nc(-c4cccc5cccc(Cl)c45)ccc23)C[C@@H]1CC#N]

On the other hand, the present invention also provides a pharmaceutical composition comprising the compound represented by formula (I) or a pharmaceutically acceptable salt thereof and a pharmaceutically acceptable excipient.

In another aspect, the present invention relates to a method for treating a disease related to KRAS G12C in a mammal, comprising administering a therapeutically effective amount of a compound represented by formula (I) or a pharmaceutically acceptable compound thereof to a mammal in need of the treatment, preferably a human being salt, or a pharmaceutical composition thereof.

In another aspect, the present invention relates to the use of the compound represented by formula (I) or a pharmaceutically acceptable salt thereof in a medicament for preventing or treating KRAS G12C-related diseases.

In another aspect, the present invention relates to a compound represented by formula (I) or a pharmaceutically acceptable salt thereof, or a pharmaceutical composition thereof for preventing or treating KRAS G12C-related diseases.

### Chemical Terms

Unless stated to the contrary, the following terms are used in the specification and claims.

“C_{x-y}” as used herein means a range of carbon atoms, where x and y are both integers, e.g. C₃₋₈ cycloalkyl means a cycloalkyl group having 3-8 carbon atoms, i.e. a cycloalkyl group having 3, 4, 5, 6, 7 or 8 carbon atoms. It should also be understood that “C₃₋₈ ” also includes any subrange therein, such as C₃₋₇, C₃₋₆, C₄₋₇, C₄₋₆, C₅₋₆, and the like.

“Alkyl” refers to a straight or chain or branched hydrocarbyl group containing 1 to 20 carbon atoms, such as 1 to 18 carbon atoms, 1 to 12 carbon atoms, 1 to 8 carbon atoms, 1 to 6 carbon atoms, or 1 to 4 carbon atoms. Non-limiting examples of alkyl groups include methyl, ethyl, n-propyl, isopropyl, n-butyl, isobutyl, tert-butyl, sec-butyl, n-pentyl, 1,1-dimethylpropyl 1,2-dimethylpropyl, 2,2-dimethylpropyl, 1-ethylpropyl, 2-methylbutyl, 3-methylbutyl, n-hexyl, 1-ethyl -2-methylpropyl, 1,1,2-trimethylpropyl, 1,1-dimethylbutyl, 1,2-dimethylbutyl, 2,2-dimethylbutyl, 1,3-dimethylbutyl and 2-ethylbutyl. The alkyl group may be substituted or unsubstituted.

“Alkenyl” refers to a straight or branched chain hydrocarbyl group containing at least one carbon-carbon double bond and usually 2 to 20 carbon atoms, such as 2 to 8 carbon atoms, 2 to 6 carbon atoms, or 2 to 4 carbon atoms. Non-limiting examples of alkenyl groups include vinyl, 1-propenyl, 2-propenyl, 1-butenyl, 2-butenyl, 3-butenyl, 2-methyl-2-propenyl, 1-butenyl, 4-pentadienyl and 1,4-butadienyl. The alkenyl group may be substituted or unsubstituted.

“Alkynyl” refers to a straight or branched chain hydrocarbon group containing at least one carbon-carbon triple bond and usually 2 to 20 carbon atoms, e.g, 2 to 8 carbon atoms, 2 to 6 carbon atoms, or 2 to 4 carbon atoms group. Non-limiting examples of alkynyl groups include ethynyl, 1-propynyl, 2-propynyl, 1-butynyl, 2-butynyl, and 3-butynyl. The alkynyl group may be substituted or unsubstituted.

“Cycloalkyl” refers to a saturated cyclic hydrocarbyl substituent containing 3 to 14 carbon ring atoms. Cycloalkyl groups may be monocarbocyclic rings, usually containing 3 to 7 carbon ring atoms. Non-limiting examples of monocyclic cycloalkyl groups include cyclopropyl, cyclobutyl, cyclopentyl, cyclohexyl, and cycloheptyl. Cycloalkyl groups may optionally be bi- or tricyclic rings fused together, such as decalinyl. The cycloalkyl may be substituted or unsubstituted.

“Heterocyclyl” , “heterocycloalkyl” and “heterocycle” refer to a stable 3-18-membered monovalent non-aromatic ring, including 2-12 carbon atoms, 1-6 atoms selected from nitrogen, oxygen and sulfur heteroatoms. Unless otherwise specified, a heterocyclyl group may be a monocyclic, bicyclic, tricyclic or tetracyclic ring system, which may contain fused, spiro or bridged ring systems, with nitrogen, carbon or sulfur selectivity on the heterocyclyl group is oxidized, the nitrogen atom can be selectively quaternized, and the heterocyclic group can be partially or fully saturated. A heterocyclyl group can be attached to the rest of the molecule by a single bond through a carbon atom or a heteroatom in the ring. A heterocyclyl group containing a fused ring may contain one or more aromatic or heteroaromatic rings, so long as the attachment to the remainder of the molecule is an atom on a non-aromatic ring. For the purposes of this application, the heterocyclyl group is preferably a stable 4-11 membered monovalent non-aromatic monocyclic or bicyclic ring containing 1-3 heteroatoms selected from nitrogen, oxygen and sulfur, more preferably a stable 4-8 membered monovalent non-aromatic monocyclic ring containing 1-3 heteroatoms selected from nitrogen, oxygen and sulfur. Non-limiting examples of heterocyclyl groups include azepanyl, azetidinyl, decahydroisoquinolyl, dihydrofuranyl, indoline, dioxolane, 1,1-Dioxo-thiomorpholinyl, imidazolidinyl, imidazolinyl, isothiazolidinyl, isoxazolidinyl, morpholinyl, octahydroindolyl, octahydroisoindolyl, oxazinyl, piper oxazinyl, piperidinyl, 4-piperidinyl, pyranyl, pyrazolidine, pyrrolidinyl, quinazinyl, quinuclidinyl, tetrahydrofuranyl, tetrahydropyranyl and the like.

“Spiroheterocyclyl” refers to a 5- to 20-membered polycyclic heterocyclic group with one atom (called a spiro atom) shared between the monocyclic rings, wherein one or more ring atoms are selected from nitrogen, oxygen or S(O)ₘ (where m is an integer from 0 to 2) and the remaining ring atoms are carbon. These may contain one or more double bonds, but none of the rings have a fully conjugated electron system preferably 6 to 14 membered, more preferably 7 to 10 membered. Spirocycloalkyl groups are classified into mono-spiroheterocyclyl, bis-spiro-heterocyclyl or poly-spiro-heterocyclyl according to the number of Spiro atoms shared between rings, preferably mono-spirocycloalkyl and bis-spirocycloalkyl. More preferably, it is a 4-membered/4-membered, 4-membered/5-membered, 4-membered/6-membered, 5-membered/5-membered or 5-membered/6-membered monospirocyclic group. Non-limiting examples of spiroheterocyclyl include:

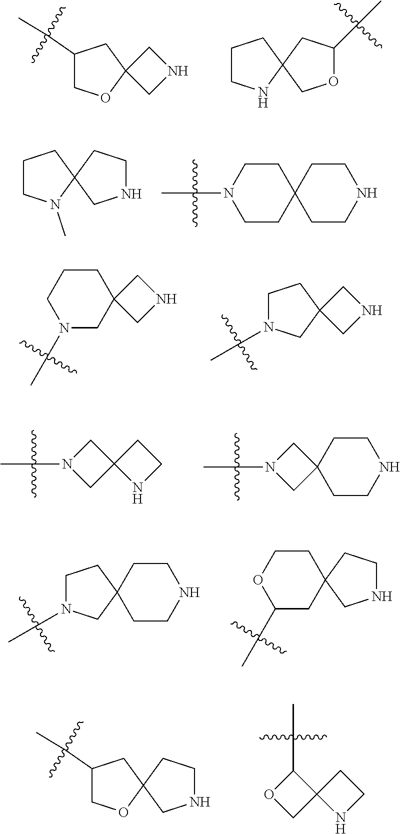

“Fused heterocyclic group” refers to a 5- to 20-membered polycyclic heterocyclic group in which each ring in the system shares an adjacent pair of atoms with other rings in the system, and one or more rings may contain one or more bicyclic bonds, but none of the rings have a fully conjugated pi electron system, where one or more ring atoms are selected from nitrogen, oxygen, or a heteroatom of S(O)ₘ (where m is an integer from 0 to 2), and the remaining ring atoms are carbon. Preferably it is 6 to 14 yuan, more preferably 7 to 10 yuan. According to the number of formed rings, it can be divided into bicyclic, tricyclic, tetracyclic or polycyclic fused heterocycloalkyl, preferably bicyclic or tricyclic, more preferably 5-membered/5-membered or 5-membered/6-membered bicyclic fused heterocyclic group. Non-limiting examples of fused heterocyclyl groups include:

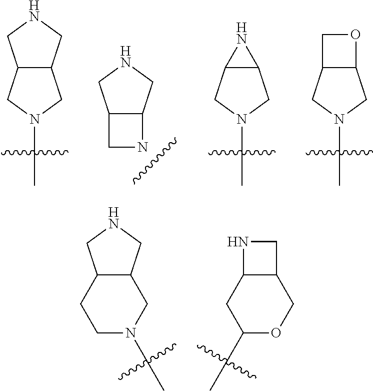

“Aryl” refers to an aromatic monocyclic or fused polycyclic group containing 6 to 14 carbon atoms, preferably 6 to 10 membered, such as phenyl and naphthyl, more preferably phenyl. The aryl ring can be fused to a heteroaryl, heterocyclyl or cycloalkyl ring, wherein the ring attached to the critical structure is an aryl ring.

“Heteroaryl” or “heteroaryl” refers to a 5-16 membered ring system comprising 1-15 carbon atoms, preferably 1-10 carbon atoms, 1-4 selected from nitrogen, oxygen and sulfur heteroatoms, at least one aromatic ring. Unless otherwise specified, a heteroaryl group may be a monocyclic, bicyclic, tricyclic or tetracyclic ring system, which may contain fused or bridged ring systems, so long as the point of attachment to the rest of the molecule is an aromatic ring atom, the Nitrogen, carbon and sulfur atoms can be selectively oxidized, and nitrogen atoms can be selectively quaternized. For the purposes of the present invention, the heteroaryl group is preferably a stable 4-11 membered monoaromatic ring containing 1-3 heteroatoms selected from nitrogen, oxygen and sulfur, more preferably a stable 5-8 membered monoaromatic ring, which contains 1-3 heteroatoms selected from nitrogen, oxygen and sulfur. Non-limiting examples of heteroaryl groups include acridinyl, azepinyl, benzimidazolyl, benzindolyl, benzodioxinyl, benzodioxinyl, benzofuranonyl, benzoyl furanyl, benzonaphthofuryl, benzopyranone, benzopyranyl, benzopyrazolyl, benzothiadiazolyl, benzothiazolyl, benzotriazolyl, furanyl, Imidazolyl, indazolyl, indolyl, oxazolyl, purinyl, pyrazinyl, pyrazolyl, pyridazinyl, pyridyl, pyrimidinyl, pyrrolyl, quinazolinyl, quinolinyl, quinine base, tetrazolyl, thiadiazolyl, thiazolyl, thienyl, triazinyl, triazolyl, etc. In the present application, the heteroaryl group is preferably a 5-8 membered heteroaryl group containing 1-3 heteroatoms selected from nitrogen, oxygen and sulfur, more preferably pyridyl, pyrimidinyl and thiazolyl. The heteroaryl group may be substituted or unsubstituted.

“Halogen” refers to fluorine, chlorine, bromine or iodine.

“Hydroxy” refers to —OH, “amino” refers to —NH₂, “amido” refers to —NHCO—, “cyano” refers to —CN, “nitro” refers to —CN, “isocyano” refers to —NC, “tri “Fluoromethyl” refers to —CF₃.

The term “heteroatom” or “hetero” as used herein, alone or as part of other components, refers to atoms other than carbon and hydrogen, the heteroatoms being independently selected from oxygen, nitrogen, sulfur, phosphorus, silicon, selenium, and tin, Without being limited to these atoms, in embodiments where two or more heteroatoms are present, the two or more heteroatoms may be the same as each other, or some or all of the two or more heteroatoms may be different.

The term “fused” or “fused ring” as used herein, alone or in combination, refers to a cyclic structure in which two or more rings share one or more bonds.

The term “spiro” or “spirocycle” as used herein, alone or in combination, refers to a cyclic structure in which two or more rings share one or more atoms.

“Optional” or “optionally” means that the subsequently described event or circumstance may but need not occur, and that the description includes instances where the event or circumstance occurs or does not occur. For example, “heterocyclyl optionally substituted with alkyl “Group” means that an alkyl group may, but need not, be present, and the description includes the case where the heterocyclic group is substituted with the alkyl group and the case where the heterocyclic group is not substituted with the alkyl group.

“Substituted” means that one or more atoms in a group, preferably 5, more preferably 1 to 3 atoms, are independently of each other substituted with the corresponding number of substituents. It goes without saying that the substituents are in their possible chemical positions, and the person skilled in the art can determine (either experimentally or theoretically) possible or impossible substitutions without undue effort. For example, carbon atoms with free amine or hydroxyl groups bound to carbon atoms with unsaturated (eg, olefinic) bonds may be unstable. The substituents include, but are not limited to, hydroxyl, amine, halogen, cyano, C₁₋₆ alkyl, C₁₋₆ alkoxy, C₂₋₆ alkenyl, C₂₋₆ alkynyl, C₃₋₈ cycloalkyl, etc.

“Pharmaceutical composition” refers to a composition comprising one or more of the compounds described herein, or a pharmaceutically acceptable salt or prodrug thereof, and other components such as pharmaceutically acceptable carriers and excipients. The purpose of the pharmaceutical composition is to facilitate the administration to the organism, facilitate the absorption of the active ingredient and then exert the biological activity.

“Isomers” refer to compounds that have the same molecular formula but differ in the nature or order in which their atoms are bonded or in the spatial arrangement of their atoms.

Stereoisomers include optical isomers, geometric isomers and conformational isomers. The compounds of the present invention may exist in the form of optical isomers. These optical isomers are of the “R” or “S” configuration, depending on the configuration of the substituents around the chiral carbon atom. Optical isomers include enantiomers and diastereomers, and methods of making and separating optical isomers are known in the art.

The compounds of the present invention may also exist as geometric isomers. The present invention contemplates various geometric isomers and mixtures thereof resulting from the distribution of substituents around carbon-carbon double bonds, carbon-nitrogen double bonds, cycloalkyl or heterocyclic groups. Substituents around a carbon-carbon double bond or carbon-nitrogen bond are designated as the Z or E configuration, and substituents around a cycloalkyl or heterocycle are designated as the cis or trans configuration.

The compounds of the present invention may also exhibit tautomerism, such as keto-enol tautomerism.

It is to be understood that the present invention includes any tautomeric or stereoisomeric form and mixtures thereof, and is not limited to any one tautomeric or stereoisomeric form used in the naming of the compounds or chemical formulae.

“Isotopes” are all isotopes of atoms occurring in the compounds of the present invention. Isotopes include those atoms with the same atomic number but different mass numbers. Examples of isotopes suitable for incorporation into the compounds of the present invention are hydrogen, carbon, nitrogen, oxygen, phosphorus, fluorine, and chlorine, such as, but not limited to, ²H, ³H , ¹³C, ¹⁴C, ¹⁵N , ¹⁸O, ³¹P, ³²P, ³⁵S, ¹⁸F, respectively, and ³⁶Cl. Isotopically labeled compounds of the present invention can generally be prepared by conventional techniques known to those skilled in the art or by methods analogous to those described in the accompanying Examples, using the appropriate isotopically labeled reagents in place of non-isotopically labeled reagents. Such compounds have various potential uses, e.g., as standards and reagents in the determination of biological activity. In the case of stable isotopes, such compounds have the potential to advantageously alter biological, pharmacological or pharmacokinetic properties.

“Prodrug” means that a compound of the present invention can be administered in the form of a prodrug. Prodrugs refer to derivatives that are converted into biologically active compounds of the present invention under physiological conditions in vivo, eg, by oxidation, reduction, hydrolysis, etc., each with or without enzymes. Examples of prodrugs are compounds in which the amine group in the compounds of the present invention is acylated, alkylated or phosphorylated, such as eicosanoylamino, propylamineamido, pivaloyloxymethylamine, or wherein the hydroxyl group is acylated, alkylated, phosphorylated or converted to a boronate salt such as acetoxy, palmitoyloxy, pivaloyloxy, succinyloxy, fumaryloxy, alanyloxy Oxygen groups, or in which the carboxyl group is esterified or amidated, or in which the sulfhydryl group forms disulfide bridges with carrier molecules such as peptides that selectively deliver drugs to the target and/or to the cytosol of cells, these compounds may be used by the present invention The compounds are prepared according to known methods.

“Pharmaceutically acceptable salt” or “pharmaceutically acceptable” means prepared from pharmaceutically acceptable bases or acids, including inorganic bases or acids and organic bases or acids. Where the compounds of the present invention contain one or more acidic or basic groups, the present invention also includes their corresponding pharmaceutically acceptable salts. Thus, the compounds of the invention which contain acidic groups can exist in salt form and can be used according to the invention, for example as alkali metal salts, alkaline earth metal salts or as ammonium salts. More precise examples of such salts include sodium, potassium, calcium, magnesium salts or with amines or organic amines such as primary, secondary, tertiary, cyclic, etc., such as ammonia, isopropylamine, trimethylamine, dimethine Particularly preferred organic bases such as ethylamine, triethylamine, tripropylamine, ethanolamine, diethanolamine, ethanolamine, dicyclohexylamine, ethylenediamine, purine, piperazine, piperidine, choline and caffeine are isopropylamine, Salts of ethylamine, ethanolamine, trimethylamine, dicyclohexylamine, choline and caffeine. The compounds of the invention which contain basic groups can exist in salt form and can be used according to the invention in the form of their additions to inorganic or organic acids. Examples of suitable acids include hydrochloric acid, hydrobromic acid, phosphoric acid, sulfuric acid, phosphoric acid, methanesulfonic acid, p-toluenesulfonic acid, naphthalenedisulfonic acid, oxalic acid, acetic acid, tartaric acid, lactic acid, salicylic acid, benzoic acid, formic acid, propylene acid, pivalic acid, malonic acid, succinic acid, pimelic acid, fumaric acid, maleic acid, malic acid, sulfamic acid, phenylpropionic acid, gluconic acid, ascorbic acid, isonicotinic acid, citric acid, adipic acid and other acids known to those skilled in the art. If the compounds of the present invention contain both acidic and basic groups in the molecule, the present invention includes, in addition to the salt forms mentioned, inner or betaine salts. The respective salts are obtained by conventional methods known to those skilled in the art, for example by contacting these with organic or inorganic acids or bases in solvents or dispersants or by anion exchange or cation exchange with other salts.

Accordingly, when referring to “a compound”, “a compound of the present invention” or “a compound of the present invention” in this application, it includes all such compound forms, such as prodrugs, stable isotope derivatives, pharmaceutically acceptable salts, Isomers, mesomers, racemates, enantiomers, diastereomers and mixtures thereof.

As used herein, the term “tumor” includes benign tumors and malignant tumors (e.g., cancer).

As used herein, the term “cancer” includes various malignancies in which KRASG¹²C mutation is involved in the occurrence, including but not limited to non-small cell lung cancer, esophageal cancer, melanoma, rhabdomyosarcoma, cell carcinoma, multiple myeloma, Breast ovarian cancer, uterine lining cancer, cervical cancer, stomach cancer, node cancer, bladder cancer, pancreatic cancer, lung cancer, breast cancer, prostate cancer and liver cancer (e.g. hepatocellular carcinoma), more particularly liver cancer, stomach cancer and bladder cancer.

As used herein, the terms “effective amount,” “therapeutically effective amount,” or “pharmaceutically effective amount” refer to at least one agent or compound sufficient to alleviate, to some extent, one or more symptoms of the disease or disorder being treated upon administration. amount. The result can be a reduction and/or remission of signs, symptoms or causes or any other desired change in a biological system. For example, an “effective amount” for treatment is that amount of a composition comprising a compound disclosed herein required to provide clinically significant relief of a condition. An effective amount appropriate in any individual case can be determined using techniques such as dose escalation assays.

As used herein, the term “polymorph” or “polymorph (phenomenon)” means that the compounds of the present invention have multiple crystal lattice forms. Some compounds of the present invention may have more than one crystal form. The present invention covers All polymorphs or mixtures thereof.

Intermediate compounds of the compounds of the present invention and polymorphs thereof are also within the scope of the present invention.

Crystallization often results in solvates of the compounds of the present invention, and the term “solvate” as used herein refers to a complex consisting of one or more molecules of the compound of the present invention in combination with one or more molecules of a solvent.

The solvent may be water, in which case the solvate is a hydrate. In addition, an organic solvent may be used. Accordingly, the compounds of the present invention may exist as hydrates, including monohydrates, dihydrates, hemihydrates, trihydrates, tetrahydrates, and the like, as well as the corresponding solvated forms. The compounds of the present invention may be true solvates, but in other cases, the compounds of the present invention may only accidentally retain water or a mixture of water and some other solvent. The compounds of the present invention may be reacted in a solvent or in a solvent Precipitation or crystallization. Solvates of the compounds of the present invention are also included within the scope of the present invention.

The term “acceptable” as used herein in relation to a formulation, composition or ingredient means no persistent detrimental effect on the general health of the subject being treated.

As used herein, the term “pharmaceutically acceptable” refers to a substance (e.g., a carrier or diluent) that does not affect the biological activity or properties of the compounds of the present invention, and is relatively non-toxic, ie, the substance can be administered to an individual without causing adverse biological effects React or interact in an undesirable manner with any component contained in the composition.

“Pharmaceutically acceptable carrier” includes, but is not limited to, adjuvants, carriers, excipients, auxiliaries, deodorants, diluents, preservatives, Dyes/colorants, flavor enhancers, surfactants and wetting agents, dispersing agents, suspending agents, stabilizers, isotonic agents, solvents, or emulsifiers.

The terms “subject”, “patient”, “subject” or “individual” as used herein refer to an individual suffering from a disease, disorder or condition, etc., including mammals and non-mammals, examples of mammals include, but are not limited to, the class of mammals Any member of: humans, non-human primates (e.g. chimpanzees and other apes and monkeys); domestic animals such as cattle, horses, sheep, goats, pigs; domestic animals such as rabbits, dogs and cats; laboratory animals, including rodents such as rats, mice and guinea pigs. Examples of non-human mammals include, but are not limited to, birds, fish, and the like. In one embodiment of the related methods and compositions provided herein, the mammal is a human.

The term “treatment” as used herein refers to the treatment of related disease conditions in mammals, particularly humans, including
preventing mammals, particularly mammals who have been previously exposed to a disease or disorder but have not been diagnosed with the disease or disorder, from developing a corresponding disease or disorder;
(ii) inhibiting the disease or disorder, i.e. controlling its development;
(iii) alleviating the disease or condition, i.e. causing regression of the disease or condition;
(iv) Relief of symptoms caused by a disease or disorder.

As used herein, the terms “disease” and “disorder” can be used interchangeably or have different meanings, because some specific diseases or conditions have no known causative agent (so the cause of the disease is not clear), so they cannot be considered as A disease can only be seen as an unwanted condition or syndrome which has more or less specific symptoms that have been confirmed by clinical researchers.

The terms “administering,” “administering,” “administering,” and the like, as used herein, refer to methods that enable the delivery of a compound or composition to the desired site of biological action. These include, but are not limited to, oral routes, duodenal routes, parenteral injection (including intravenous, subcutaneous, intraperitoneal, intramuscular, intra arterial injection or infusion), topical administration, and rectal administration. In preferred embodiments, the compounds and compositions discussed herein are administered orally.

### Specific Implementation Method

The present invention also provides methods for preparing the compounds. The preparation of the compounds of the general formula (I) of the present invention can be accomplished by the following exemplary methods and examples, but these methods and examples should not be construed as limiting the scope of the present invention in any way. The compounds of the present invention can also be synthesized using synthetic techniques known to those skilled in the art, or a combination of methods known in the art and methods described in the present invention. The products obtained in each step are obtained by separation techniques known in the art, including but not limited to extraction, filtration, distillation, crystallization, chromatographic separation, and the like. The starting materials and chemical reagents required for the synthesis can be routinely synthesized or purchased according to the literature (reaxys).

The compound described in the general formula (I) of the present invention or its isomer, solvate or its precursor, or their pharmaceutically acceptable salt is as follows:

### Method A

The starting material A1 and the precursor H-U-Y-P undergo an aromatic nucleophilic substitution reaction under the action of a base to generate A2;

2 [symbol] A3 and precursor R₁ undergo aromatic nucleophilic substitution reaction under the action of alkali to form A3;

3 [symbol] A3 is coupled to obtain intermediate A4;

4 [symbol] A4 removes the protecting group to obtain A5;

5 [symbol] The amine group in A5 is treated with a chemical reagent (e.g., acryloyl chloride, etc.) containing a functional group that reacts with a cysteine residue in the ligand binding domain of the kinase to obtain the compound of general formula (I).

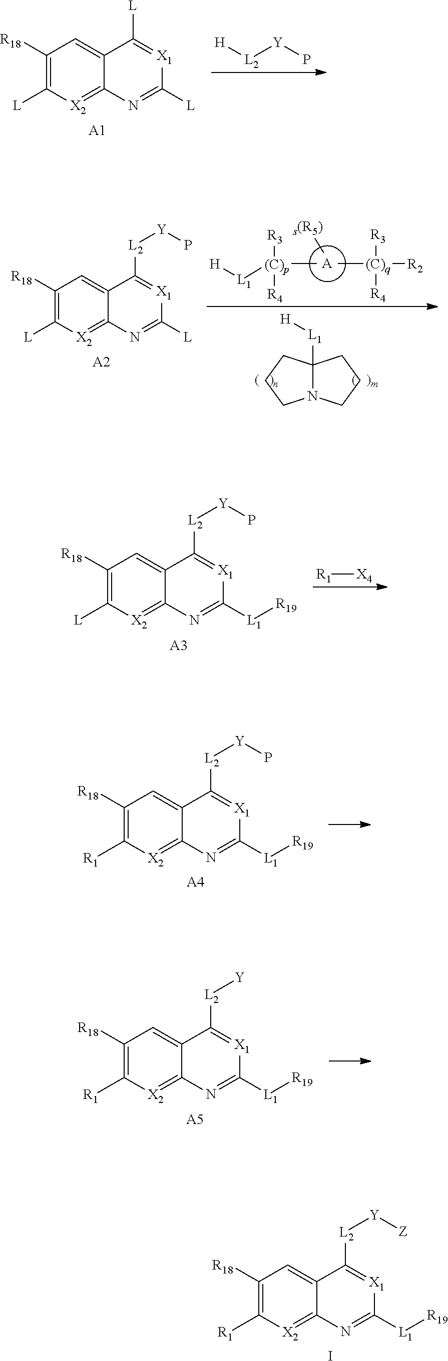

### Method B

1 [symbol] Starting material B1 and intermediate B2 are obtained by coupling;

2 [symbol] B2 and POCl₃ or POBr₃ obtain intermediate B3;

3 [symbol] B3 and the precursor H-U-Y-P undergo aromatic nucleophilic substitution reaction under the action of alkali to form B4;

4 [symbol] B4 and precursor R₁ undergo aromatic nucleophilic substitution reaction under the action of alkali to form B5;

5 [symbol] B5 removes the protecting group to obtain A5;

The compound of general formula (I) is then prepared by the method.

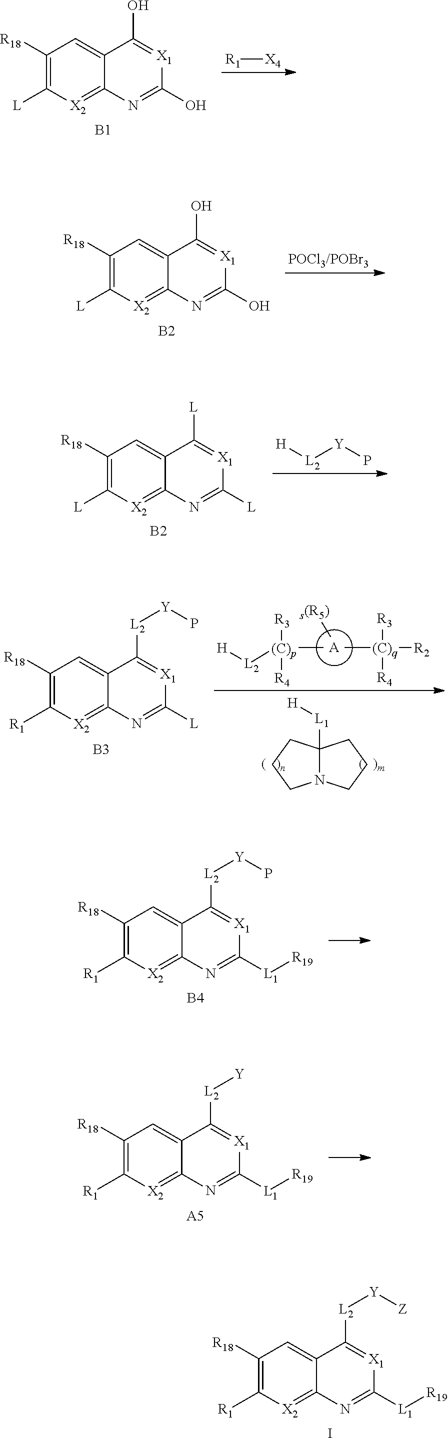

Unless otherwise stated, temperatures are in degrees Celsius. Reagents were purchased from commercial suppliers such as Chem blocks Inc, Astatech Inc or Maclean and these reagents were used without further purification unless otherwise stated.

Unless otherwise stated, the following reactions were performed at room temperature, in anhydrous solvent, under positive pressure of nitrogen or gas, or using a drying tube; glassware drying and/or heat drying.

Unless otherwise stated, column chromatography used 200-300 mesh silica gel from Qingdao Ocean Chemical Factory; preparative thin layer chromatography used thin layer chromatography silica gel prefabricated plate (HSGF254) produced by Yantai Chemical Industry Research Institute; MS was measured with Therno LCD Fleet type (ESI) liquid chromatography-mass spectrometer.

Nuclear magnetic data (¹H NMR) use Bruker Avance-400 MHz or Varian Oxford-400 Hz nuclear magnetic instrument, the solvents used in nuclear magnetic data are CDCl₃, CD₃OD, D₂O, DMSO-d₆, etc., based on tetramethylsilane (0.000 ppm) or residual Solvent-based (CDCl₃: 7.26 ppm; CD₃OD: 3.31 ppm; D₂O: 4.79 ppm; d₆-DMSO: 2.50 ppm) When the peak shape diversity is indicated, the following abbreviations indicate different peak shapes: s (single peak), d (double peak) peak), t (triplet), q (quartet), m (multiplet), br (broad), dd (double doublet), dt (double triplet). If a coupling constant is given, it is in Hertz (Hz).

### Methods of Preparation

### 7-chloro-6-fluoropyrido[2,3-d]pyrimidine-2,4(1H,3H)-dione

A mixture of 2,6-dichloro-5-fluoronicotinic acid (54.6 g, 260 mmol) and 2N NaOH (625 ml) was stirred at reflux for 2 hours, then at 128° C. for 6 hours. The reaction mixture was cooled to 0° C. and acidified with 6N HCl. The mixture was cooled in an ice bath for 30 minutes, the solids were filtered and washed with H2O. The isolated solid was slurried in warm ethanol, filtered, and washed with warm ethanol. The solid was collected and dried under vacuum overnight to give the desired product 6-hydroxy-2-chloro-5-fluoronicotinic acid (43 g, 87%) as a beige solid. LC/MS(ESI): m/z=192 [M+H]+.

6-Hydroxy-2-chloro-5-fluoronicotinic acid (40.1 g, 210 mmol) and thionyl chloride (200 mL) were added to a round-bottomed baking flask, and the mixture was stirred and refluxed for 4 hours. The reaction mixture was cooled to room temperature and concentrated under reduced pressure. Then 250 mL of anhydrous methanol was added, concentrated under reduced pressure, and slurried in water to obtain methyl 6-hydroxy-2-chloro-5-fluoronicotinate (42.4 g, 98%). LC/MS(ESI): m/z=207 [M+H]+.

Add methyl 6-hydroxy-2-chloro-5-fluoronicotinate (41.8 g, 200 mmol) to a round-bottomed baking bottle, dissolve in 500 mL of DMF, add p-methoxybenzylamine (32.9 g, 240 mmol), potassium iodide (2 g) and cuprous iodide (1 g), and the reaction mixture was stirred at reflux for 6 hours. The reaction mixture was cooled to room temperature, poured into water, filtered to obtain the crude product, and slurried in water to obtain methyl 6-hydroxy-2-p-methoxybenzylamino-5-fluoronicotinate (47.7 g, 78%). LC/MS (ESI): m/z=307 [M+H]+.

Under nitrogen protection at 27° C., methyl 6-hydroxy-2-p-methoxybenzylamino-5-fluoronicotinate (45.9 g, 150 mmol) was dissolved in EtOH (300 mL), and 10% Pd was added proportionally/C (11.2 g). The reaction mixture was purged with hydrogen and then stirred under balloon pressure hydrogen for 16 hours. Upon completion, the reaction mixture was filtered through a small pad of celite, and the filter cake was extracted with DCM (200 mL). The filtrate was concentrated under reduced pressure to obtain crude product. The crude product was purified by silica gel column chromatography to give methyl 6-hydroxy-2-amino-5-fluoronicotinate (27.3 g, 98% yield) as a yellow solid. LC/MS (ESI): m/z=187 [M+H]+.

6-Hydroxy-2-amino-5-fluoronicotinic acid methyl ester (26.0 g 140 mmol) was dissolved in POCl 3 (250 mL), 10 mL of N,N-dimethylaniline was added, and the reaction was stirred under reflux for 10 h. Then poured into ice water to quench, filtered to obtain solid product, washed with water, and dried to obtain crude yellow solid 6-chloro-2-amino-5-fluoronicotinic acid methyl ester (24.1 g, 84%), and the next step was carried out without further purification. reaction. LC/MS (ESI): m/z=206 [M+H]+.

Methyl 6-chloro-2-amino-5-fluoronicotinate (22.6 g, 110 mmol) was added to 2N lithium hydroxide (250 mL) in a round-bottomed baking flask, the mixture was stirred at room temperature for 6 hours, and the reaction mixture was washed with 6N hydrochloric acid. The pH was adjusted to 7, then the solid was filtered and slurried in water to give 6-chloro-2-amino-5-fluoronicotinic acid (16.6 g, 79%). LC/MS (ESI): m/z=192 [M+H]+.

6-Chloro-2-amino-5-fluoronicotinic acid (15.2 g, 80 mmol) and thionyl chloride (100 mL) were added to a round-bottomed baking flask, and the mixture was stirred and refluxed for 4 hours. The reaction mixture was cooled to room temperature and concentrated under reduced pressure. Then, 100 mL of anhydrous tetrahydrofuran was added, ammonia gas was introduced, the mixture was stirred at room temperature for 2 hours, and concentrated under reduced pressure to obtain 6-chloro-2-amino-5-fluoronicotinamide (14.7 g, 97%). LC/MS (ESI): m/z=191 [M+H]+.

Under nitrogen, 6-chloro-2-amino-5-fluoronicotinamide (13.3 g 70 mmol) was added to 150 m dry toluene, followed by dropwise addition of oxalyl chloride (1.2 eq). The resulting mixture was then heated to reflux (115° C.), cooled for 4 hours, and then stirred for an additional 16 hours. The crude reaction mixture was then concentrated in vacuo to half its volume and filtered to give 7-chloro-6-fluoro-pyrido[2,3-d]pyrimidine-2,4(1H,3H)-dione (13.9 g, 92%), the resulting product did not require any further purification. LC/MS (ESI): m/z=217 [M+H]+.

### 2,4,7-trichloro-6-fluoropyridine[2,3-d]lopyrimidine

A mixture of 2,6-dichloro-5-fluoronicotinamide (54.6 g, 260 mmol) and 2N NaOH (625 ml) was stirred at reflux for 2 hours, then at 128° C. for 6 hours. The reaction mixture was cooled to 0° C. and acidified with 6N HCl. The mixture was cooled in an ice bath for 30 minutes, the solids were filtered and washed with H₂O. The isolated solid was slurried in warm ethanol, filtered, and washed with warm ethanol. The solid was collected and dried under vacuum overnight to give the desired product 6-hydroxy-2-chloro-5-fluoronicotinamide (42.3 g, 78%) as a tan solid. LC/MS (ESI): m/z=210 [M+H]+.

6-Hydroxy-2-chloro-5-fluoronicotinamide (42 g, 200 mmol) was added to a round-bottomed baking bottle, dissolved in 500 mL DMF, p-methoxybenzylamine (32.9 g, 240 mmol), potassium iodide (2 g) and iodide were added. cuprous (1 g), and the reaction mixture was stirred at reflux for 6 hours. The reaction mixture was cooled to room temperature, poured into water, and filtered to obtain crude product, which was slurried in water to obtain 6-hydroxy-2-p-methoxybenzylamino-5-fluoronicotinamide (44.1 g, 76%). LC/MS (ESI): m/z=291 [M+H]+.

Under nitrogen protection at 27° C., 6-hydroxy-2-p-methoxybenzylamino-5-fluoronicotinamide (43.5 g, 150 mmol) was dissolved in EtOH (300 mL), and 10% Pd/C was added proportionally (11.2 g). The reaction mixture was purged with hydrogen and then stirred under balloon pressure hydrogen for 16 hours. Upon completion, the reaction mixture was filtered through a small pad of celite, and the filter cake was extracted with DCM (200 mL). The filtrate was concentrated under reduced pressure to obtain crude product. The crude product was purified by silica gel column chromatography to give 6-hydroxy-2-amino-5-fluoronicotinamide as a yellow solid (25 g, 97% yield). LC/MS (ESI): m/z=172 [M+H]+.

Under nitrogen protection, 2-amino-6-hydroxy-5-fluoronicotinamide (23.9 g, 140 mmol, 1 eq) was added to 200 mL of dry toluene, followed by dropwise addition of oxalyl chloride (1.2 eq). The resulting mixture was then heated to reflux for 4 hours, then cooled and stirred for an additional 16 hours. The crude reaction mixture was then concentrated in vacuo to half its volume and filtered to give 7-hydroxy-6-fluoro-pyrido[2,3-d]pyrimidine-2,4(1H,3H)-dione (26.2 g 95%), the resulting product did not require any further purification. LC/MS (ESI): m/z=198 [M+H]+.

7-Hydroxy-6-fluoro-pyrido[2,3-d]pyrimidine-2,4(1H,3H)-dione (23.8 g 120 mmol) was dissolved in POCl3 (250 mL), 10 mL N,N- The xylene amine was heated under reflux and stirred for 10 h. Then poured into ice water to quench, filtered to obtain solid product, washed with water and dried to obtain crude yellow solid 6-fluoro-2,4,7-trichloropyrido[2,3-d]pyrimidine (24.7 g, 82%), The next reaction was carried out without further purification. LC/MS(ESI): m/z=253 [M+H]+.

### 3-(dimethylamino)cyclobutan-1-ol

HCHO (6 mL) and HCOOH (6 mL) were mixed with 3-aminocyclobutanol (600 mg, 6.89 mmol) at room temperature and heated to reflux for 3 h. Condensation reduction afforded the crude product, which was dissolved in methanol (40 mL) and treated with Dowex 50WX4 (type H) resin for 30 minutes. The resin was then filtered and washed with methanol (2×20 mL). After this time, the resin was eluted with 7.0 M NH₃ in MeOH (200 mL) to give 3-(dimethylamino)cyclobutanol (221 mg, 28% yield, two steps) as a pale yellow sticky substance. LC/MS(ESI): m/z=116 [M+H]+.

### 1-(dimethylaminomethyl)cyclopropane-1-methanol

HCHO (6 mL) and HCOOH (6 mL) were mixed with 3-aminocyclobutanol (600 mg, 6.89 mmol) at room temperature and heated to reflux for 3 h. Condensation reduction afforded the crude product, which was dissolved in methanol (40 mL) and treated with Dowex 50WX4 (type H) resin for 30 minutes. The resin was then filtered and washed with methanol (2×20 mL). After this time, the resin was eluted with 7.0M NH3 in MeOH (200 mL) and reduced distillation to give 1-(dimethylaminomethyl)cyclopropane-1-methanol (270 mg, 35% yield, two steps). LC/MS(ESI): m/z=130 [M+H]+.

### 1-(1-pyrrolidinylmethyl)cyclopropane-1-methanol

A solution of oxalyl chloride (12.5 mL, 2M) in dichloromethane was added to methyl cyclopropane-1,1-dicarboxylate (2.90 g, 20 mmol) in dichloromethane (50 mL) under ice/water bath cooling with stirring To the solution, DMF (100 μl) was then added and the reaction was stirred for about 2 hours. At room temperature, a pale yellow solution was obtained. The solution was concentrated to a yellow semisolid. Under ice/water cooling and stirring, the yellow semi-solid was dissolved in (20 mL of THF, then pyrrolidine (6 mL, 71 mmol) was slowly added and stirred for about 60 min. Ethyl acetate (150 mL) was added, water (2×75 mL) and saturated chlorinated The organic phase was washed with aqueous sodium solution (75 mL). The organic phase was dried over anhydrous magnesium sulfate and concentrated to give methyl 1-(pyrrolidin-1-acyl)-cyclopropanecarboxylate (2.0 g, 50%) as a yellow-brown oil.

A solution of lithium aluminum hydride in THF (20 mL, 1 M) was slowly added to 25 mL of THF in methyl 1-(pyrrolidin-1-acyl)-cyclopropanecarboxylate (2.0 g, 10 mmol) under nitrogen blanket and ice/water bath The solution was then warmed to room temperature, and the resulting solution was stirred and reacted for 3 hours. The solution was cooled in an ice/water bath and sodium sulfate decahydrate (4.9 g, 15 mmol) was added portionwise to give a white suspension. Diethyl ether (25 mL) was added and the suspension was stirred for about 18 hours. in room temperature. The resulting suspension was filtered through celite and the solids were washed with ether (2×50 mL). The combined filtrates were concentrated, and the residue was separated and purified by silica gel column chromatography (eluent: ethyl acetate: petroleum ether=1:20≈1:1) to obtain yellow oily compound 1-(pyrrolidin-1-ylmethyl) Cyclopropyl-1-methanol (1.15 g, 64%). LC/MS(ESI): m/z=156 [M+H]+.

(S)-1-(Methyl-d3)pyrrolidine-2-methanol

Deuterated iodomethane (1.44 g, 0.01 mol), (S)-2-((((tert-butyldiphenylsilyl)oxy)methyl)pyrrolidine (3.74 g, 0.011 mol), carbonic acid Potassium (2.2 g, 0.02 mmol) and DMF (200 mL) were mixed, heated to 120° C., and stirred for 4 hours. Cooled to room temperature, evaporated under reduced pressure to obtain yellow filtration and concentrated under reduced pressure to obtain (S)-2-((((tert-butyldiphenylsilyl)oxy)methyl)-1-(methyl-d3)pyrrolidine (3.11 g, 88% yield).

CsF (1.75 g, 1.5 mmol) was added to (S)-2-((((tert-butyldiphenylsilyl)oxy)methyl)-1-(methyl-d3)pyrrolidine (1.4 g, 3.95 mmol) in DMF (15 mL) and stirred at 50° C. for 20 h. The reaction mixture was then cooled to room temperature, diluted with H₂O₂O (20 mL) and extracted with ethyl acetate (3×30 mL). Washed with 80 mL of saturated brine The combined organic layers were dried over anhydrous Na2SO4 and filtered. The filtrate was concentrated under reduced pressure. The residue was purified by column chromatography (petroleum ether/ethyl acetate, 10:1 to 1:1) to give (S) as a pale yellow oil-1-(Methyl-d3)pyrrolidine-2-methanol (270 mg, 58% yield). LC/MS(ESI): m/z=119 [M+H]+.

### 8-fluoronaphthalene Boronic Acid

At 0° C., 48% HBF4 (100 mL) was added to a solution of 8-bromo-1-naphthylamine (10 g, 45.2 mmol) in 100 mL of THF followed by NaNO2 (4.9 g, 135.8 mmol) in 20 mL of water solution. The reaction was stirred at 0° C. for 1 h, then NaBF4 (24.9 g, 226 mmol). The mixture was warmed to room temperature and filtered. The solid was washed with ether and dried under high vacuum overnight to give the diazonium salt as a green solid, the diazonium salt obtained in the previous step was suspended in xylene (50 mL) and refluxed for 1 h. The filtrate was concentrated under reduced pressure, and the residue was purified by column chromatography to give 8-bromo-1-fluoronaphthalene (4.6 g, 45%).

Dissolve 8-bromo-1-fluoronaphthalene (2.79 g, 0.0124 mol) in anhydrous tetrahydrofuran (20.0 mL), add triisopropyl borate (2.68 g, 0.0142 mol), cool to −78° C., add n-butyl base lithium (0.95 g, 0.0149 mol), and the reaction was stirred for 0.5 h, then returned to room temperature. The reaction was quenched by adding saturated aqueous ammonium chloride solution. The pH was adjusted to strong acidity, and extracted with ethyl acetate (20.0 ml×3). The organic phases were combined, washed with saturated brine, dried over anhydrous sodium sulfate, and the solvent was distilled off under reduced pressure. Slurry with n-hexane and filter to give 8-fluoronaphthalene-1-boronic acid (1.98 g, 84%). LC/MS(ESI): m/z=191 [M+H]+.

### EXAMPLE 1

7-(2-Fluoro-6-hydroxyphenyl)-4-(((R)-4-acryloyl-2-methylpiperazine)-1-yl)-2-(((S)-1-methylpyrrolidin-2-yl)methoxy)-1,8-naphthyridine (compound 1)

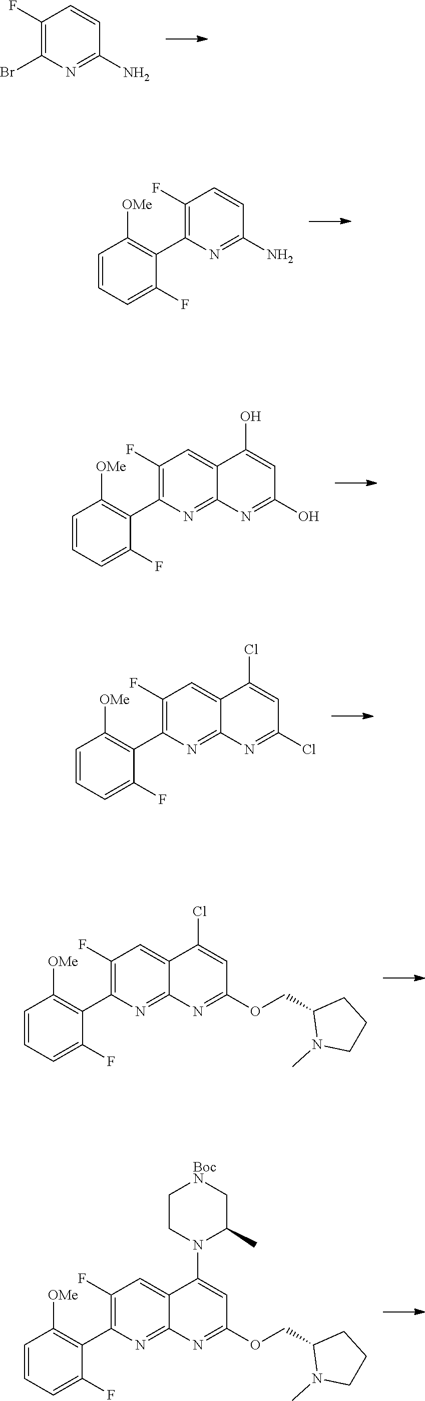 [SMILES: Nc1ccc(F)c(Br)n1 ; COc1cccc(F)c1-c1nc(N)ccc1F ; COc1cccc(F)c1-c1nc2nc(O)cc(O)c2cc1F ; COc1cccc(F)c1-c1nc2nc(Cl)cc(Cl)c2cc1F ; COc1cccc(F)c1-c1nc2nc(OC[C@@H]3CCCN3C)cc(Cl)c2cc1F ; COc1cccc(F)c1-c1nc2nc(OC[C@@H]3CCCN3C)cc(N3CCN(C(=O)OC(C)(C)C)C[C@H]3C)c2cc1F]

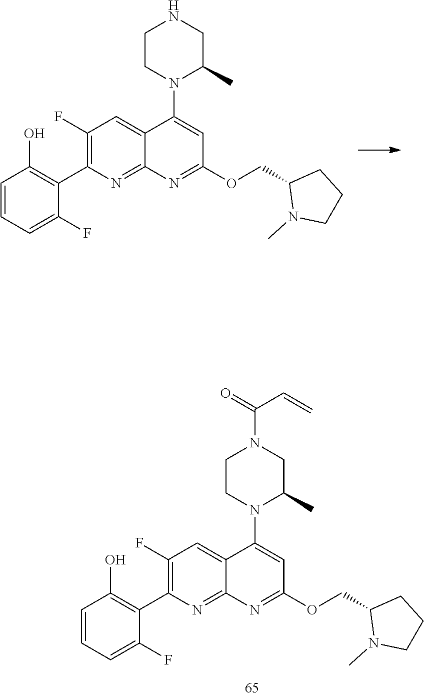 [SMILES: C[C@@H]1CNCCN1c1cc(OC[C@@H]2CCCN2C)nc2nc(-c3c(O)cccc3F)c(F)cc12 ; C=CC(=O)N1CCN(c2cc(OC[C@@H]3CCCN3C)nc3nc(-c4c(O)cccc4F)c(F)cc23)[C@H](C)C1]

### Step 1: 2-(2-fluoro-6-methoxyphenyl)-3-fluoro-6-aminopyridine

Combine 6amino-2-bromo-3-fluoropyridine (9.55 g, 0.05 mol), 6-methoxy-2-fluorophenylboronic acid (10.7 g, 0.05 mo ), tris(dibenzylideneacetone)dipalladium (4 g, 4.4 mmol), cesium carbonate, 1,4-dioxane (500 mL) and water (100 mL) were mixed, then heated to 120° C. under reflux, and the reaction was stirred for 16 hours. The reaction was cooled to room temperature and stirred overnight to give a pale yellow precipitate. The reaction mixture was diluted with water (10 mL) and the solid was collected by filtration. The crude product was slurried with methanol (50 mL) to give 2-(2-fluoro-6-methoxyphenyl)-3-fluoro-6-aminopyridine (7.22 g, 61%) as a yellow solid, which was carried on to the next step without further purification. reaction. LC/MS(ESI): m/z=237 [M+H]+

### Step 2: 7-(2-fluoro-6-methoxyphenyl)-6-fluoro-2,4-dihydroxy-1,8-naphthyridine

2-(2-Fluoro-6-methoxyphenyl)-3-fluoro-6-aminopyridine (7.11 g, 30 mmol) and diethyl malonate (5.51 g, 3.3 mmol) were suspended in diphenyl ether (30 mL), the reaction was heated at 150° C. for 0.5 h, where the reactants became a homogeneous solution. The reaction was then refluxed for 2 hours, then cooled to room temperature, poured into water (300 mL) and extracted with ethyl acetate (300 mL). The organic phase was dried over anhydrous MgSO4, filtered and concentrated. The residue was heated under reduced pressure at 220° C. for 2 hours, and the mixture solidified. The reaction was cooled to room temperature and slurried with ethanol to give 7-(2-fluoro-6-methoxyphenyl)-6-fluoro-2,4-dihydroxy-1,8-naphthyridine (4.65 g, 51%) as a yellow solid). LC/MS(ESI): m/z=305 [M+H]+

### Step 3: 7-(2-fluoro-6-methoxyphenyl)-6-fluoro-2,4-dichloro-1,8-naphthyridine

7-(2-Fluoro-6-methoxyphenyl)-6-fluoro-2,4-dihydroxy-1,8-naphthyridine (1.83 g, 6 mmol) was dissolved in POCl₃ (30 mL), a small amount of N was added, N-Xylidine, heated and refluxed for 10 h with stirring. Then poured into ice water to quench, filtered to obtain solid product, washed with water, and dried to obtain crude yellow solid 7-(2-fluoro-6-methoxyphenyl)-6-fluoro-2,4-dichloro-1,8-naphthyridine (1.45 g, 71%), proceeded to the next reaction without further purification. LC/MS(ESI): m/z=342 [M+H]+.

### Step 4: 7-(2-Fluoro-6-methoxyphenyl)-6-fluoro-2-(((S)-1-methylpyrrolidin-2-yl)methoxy)-4-chloro-1,8-naphthyridine

7-(2-Fluoro-6-methoxyphenyl)-6-fluoro-2,4-dichloro-1,8-naphthyridine (1.37 g, 4 mmol), N-methyl-L-prolinol (0.88 g, 4.4 mmol), potassium carbonate (0.88 g, 6.4 mmol) catalytic potassium iodide and DMF (80 mL) were mixed, heated to 120° C., and the reaction was stirred for 4 hours. Cool to room temperature and evaporate under reduced pressure to obtain a yellow solid 7-(2-fluoro-6-methoxyphenyl)-6-fluoro-2-(((S)-1-methylpyrrolidin-2-yl) methoxy)-4-chloro-1,8-naphthyridine (1.16 g, 69%), LC/MS (ESI): m/z=421 [M+H]+.

### Step 5: 7-(2-Fluoro-6-methoxyphenyl)-6-fluoro-2-(((S)-1-methylpyrrolidin-2-yl)methoxy)-4-(((R)-4-boc-2-methylpiperazine)-1-yl)-1,8-naphthyridine

(R)-4-Boc-2-methylpiperazine (0.44 g, 2.2 mmol), 7-(2-fluoro-6-methoxyphenyl)-6-fluoro-2-(((S)-1-Methylpyrrolidin-2-yl)methoxy)-4-chloro-1,8-naphthyridine (0.842 g, 2 mmol), cesium carbonate (1.3 g, 4 mmol) and BINAP (0.124 g, 0.2 mmol) Dissolved in 1.4-dioxane (25 mL), the mixture was degassed by bubbling nitrogen for 5 min. To the reaction was added tris(dibenzylideneacetone)dipalladium (0.1 g, 0.11 mmol) and the reaction mixture was stirred at reflux for 24 hours. After completion of the reaction, the reaction mixture was diluted with ethyl acetate (750 mL), washed with water (100 mL), washed with brine (10 mL), and dried over anhydrous sodium sulfate. Concentration under reduced pressure gave the crude product as a brown solid, which was passed through silica gel containing ethyl acetate (900 mL) to remove any inorganics. It was then recrystallized from acetonitrile to give 7-(2-fluoro-6-methoxyphenyl)-6-fluoro-2-(((S)-1-methylpyrrolidin-2-yl)methoxy)-4-(((R)-4-boc-2-methylpiperazine)-1-yl)-1,8-naphthyridine (0.99 g, 85%). LC/MS(ESI): m/z=584.2 [M+H]+.

### Step 6: 7-(2-Fluoro-6-hydroxyphenyl)-6-fluoro-2-(((S)-1-methylpyrrolidin-2-yl)methoxy)-4-(((R) 2-methylpiperazine)-1-yl)-1,8-naphthyridine

7-(2-Fluoro-6-methoxyphenyl)-6-fluoro-2-(((S)-1-methylpyrrolidin-2-yl)methoxy)-4-((((R)-4-boc-2-methylpiperazine)-1-yl)-1,8-naphthyridine (584 mg, 1 mmol) was dissolved in 10 mL of DCM, at −78° C., BBr (0.8 mL) was added, then liters The reaction was stirred to room temperature overnight. The reaction was quenched with water and extracted with DCM (2*15 mL), the organic layers were combined, dried over anhydrous sodium sulfate, and concentrated to obtain the target product as a yellow solid 7-(2-fluoro-6-hydroxyphenyl)-6-fluoro-2-(((S)-1-Methylpyrrolidin-2-yl)methoxy)-4-(((R)2-methylpiperazine)-1-yl)-1,8-naphthyridine (365 mg, 78%). LC/MS (ESI): m/z=470.2 [M+H]+.

### Step 7: 7-(2-Fluoro-6-hydroxyphenyl)-4-((((R)-4-acryloyl-2-methylpiperazine)-1-yl)-2-((((S)-1-methylpyrrolidin-2-yl)methoxy)-1,8-naphthyridine

7-(2-Fluoro-6-hydroxyphenyl)-6-fluoro-2-(((S)-1-methylpyrrolidin-2-yl)methoxy)-4-(((R)₂-Methylpiperazine)-1-yl)-1,8-naphthyridine (235 mg, 0.5 mmol), triethylamine (81 mg, 0.8 mmol), 20 ml of tetrahydrofuran, cooled in an ice-water bath and slowly added dropwise Acryloyl chloride (72 mg, 0.8 mmol) in 0.5 ml of tetrahydrofuran. After the addition was complete, stifling was continued for 4 hours. The reaction solution was quenched with methanol and evaporated to dryness under reduced pressure. The residue was purified by column chromatography to give 7-(2-fluoro-6-hydroxyphenyl)-4-(((R)-4-acryloyl-2-methylpiperazine)-1-yl)-2-(((S)-1-methylpyrrolidin-2-yl)methoxy)-1,8-naphthyridine (107 mg, 42% yield) was a yellow solid. LC/MS(ESI): m/z=524.2 [M+H]+.

### EXAMPLE 2

7-(2-Fluoro-6-hydroxyphenyl)-6-fluoro-4-(((R)-4-(2-fluoroacryloyl)-2-methylpiperazine)-1-yl)-2-(((S)-1-methylpyrrolidin-2-yl)methoxy)-1,8-naphthyridine (Compound 2)

Compound 2 was obtained in a similar manner to Example 1(110 mg, 41% yield). LC/MS (ESI): m/z=542.2 [M+H]+.

### EXAMPLE 3

7-(2-Fluoro-6-hydroxyphenyl)-6-fluoro-4-((((S)-4-acryloyl-3-carbonitrileethylpiperazine)-1-yl)-2-((((S)-1-methylpyrrolidin-2-yl)methoxy)-1,8-naphthyridine (Compound 3)

Compound 3 was obtained in a similar manner to Example 1(126 mg, 46% yield). LC/MS (ESI): m/z=549.2 [M+H]+.

### EXAMPLE 4

7-(2-Fluoro-6-hydroxyphenyl)-6-fluoro-4-(((S)-4-(2-fluoroacryloyl)-3-carbonitrileethylpiperazine)-1-yl)-2-(((S)-1-methylpyrrolidin-2-yl)methoxy)-1,8-naphthyridine (Compound 4)

Compound 4 was obtained in a similar manner to Example 1 (96 mg, 34% yield). LC/MS (ESI): m/z=567.2 [M+H]+.

### EXAMPLE 5

7-(8-Fluoronaphthyl)-6-fluoro-4-(((S)-4-(2-fluoroacryloyl)-3-carbonitrileethylpiperazine)-1-yl)-2-((((S)-1-methylpyrrolidin-2-yl)methoxy)-1,8-naphthyridine (Compound 5)

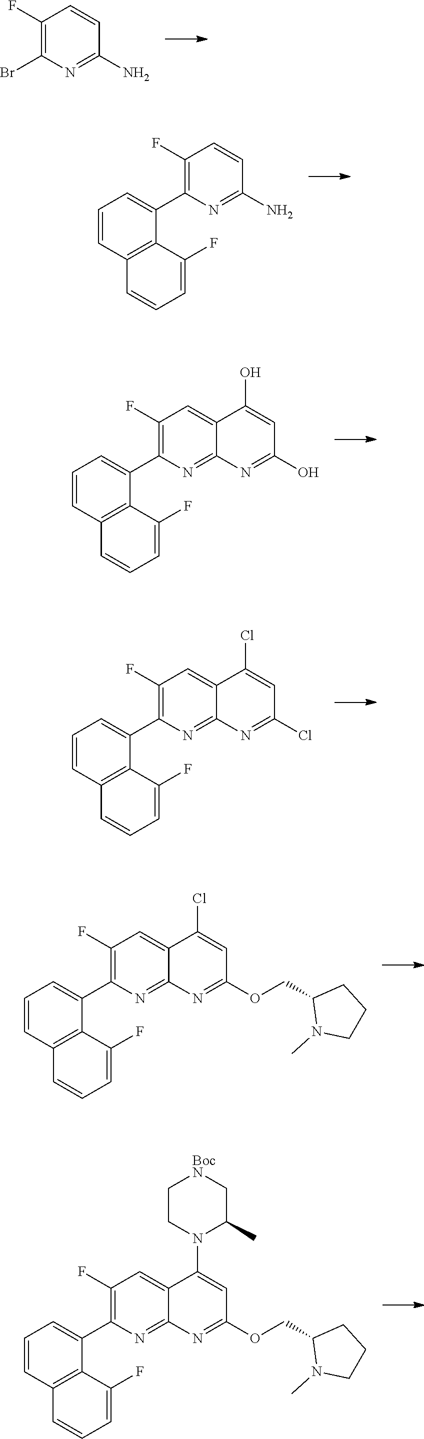 [SMILES: Nc1ccc(F)c(Br)n1 ; Nc1ccc(F)c(-c2cccc3cccc(F)c23)n1 ; Oc1cc(O)c2cc(F)c(-c3cccc4cccc(F)c34)nc2n1 ; Fc1cc2c(Cl)cc(Cl)nc2nc1-c1cccc2cccc(F)c12 ; CN1CCC[C@H]1COc1cc(Cl)c2cc(F)c(-c3cccc4cccc(F)c34)nc2n1 ; C[C@@H]1CN(C(=O)OC(C)(C)C)CCN1c1cc(OC[C@@H]2CCCN2C)nc2nc(-c3cccc4cccc(F)c34)c(F)cc12]

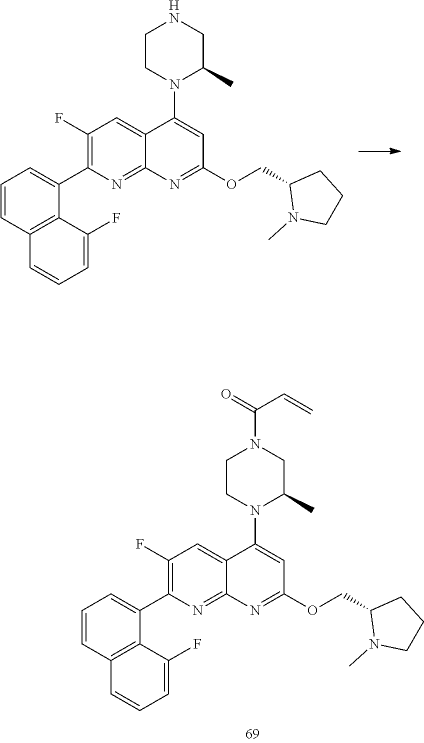 [SMILES: C[C@@H]1CNCCN1c1cc(OC[C@@H]2CCCN2C)nc2nc(-c3cccc4cccc(F)c34)c(F)cc12 ; C=CC(=O)N1CCN(c2cc(OC[C@@H]3CCCN3C)nc3nc(-c4cccc5cccc(F)c45)c(F)cc23)[C@H](C)C1]

### Step 1: 2-(8-fluoronaphthyl)-3-fluoro-6-aminopyridine

6-amino-2-bromo-3-fluoropyridine (9.55 g, 0.05 mol ), 8-fluoronaphthaleneboronic acid (12.8 g, 0.05 mol), tris(dibenzylideneacetone)dipalladium (4 g, 4.4 mmol), Cesium carbonate, 1,4-dioxane (500 mL) and water (100 mL) were mixed, then heated to 120° C. under reflux, and the reaction was stirred for 16 hours. The reaction was cooled to room temperature and stirred overnight to give a pale yellow precipitate. The reaction mixture was diluted with water (10 mL) and the solid was collected by filtration. The crude product was slurried with methanol (50 mL) to give 2-(8-fluoronaphthyl)-3-fluoro-6-aminopyridine (8.7 g, 68%) as a beige solid which was carried to the next reaction without further purification. LC/MS(ESI): m/z=257 [M+H]+.

### Step 2: 7-(8-fluoronaphthyl)-6-fluoro-2,4-dihydroxy-1,8-naphthyridine

2-(8-Fluoronaphthyl)-3-fluoro-6-aminopyridine (7.5 g, 30 mmol) and diethyl malonate (5.51 g, 3.3 mmol) were suspended in diphenyl ether (30 mL) in The reaction was heated at 150° C. for 0.5 hours, where the reactants became a homogeneous solution. The reaction was then refluxed for 2 hours, then cooled to room temperature, poured into water (300 mL) and extracted with ethyl acetate (300 mL). The organic phase was dried over anhydrous MgSO₄, filtered and concentrated. The residue was heated under reduced pressure at 220° C. for 2 hours, and the mixture solidified. The reaction was cooled to room temperature and slurried with ethanol to give 7-(8-fluoronaphthyl)-6-fluoro-2,4-dihydroxy-1,8-naphthyridine (4.94 g, 54%) as a yellow solid. LC/MS(ESI): m/z=325 [M+H]+

### Step 3: 7-(8-fluoronaphthyl)-6-fluoro-2,4-dichloro-1,8-naphthyridine

Dissolve 7-(8-fluoronaphthyl)-6-fluoro-2,4-dihydro xy-1,8-naphthyridine (1.83 g 6 mmol) in POCl3 (30 mL), add a small amount of N,N-xylidine , heated under reflux and stirred for 10 h. Then poured into ice water to quench, filtered to obtain solid product, washed with water, and dried to obtain crude yellow solid 7-(8-fluoronaphthyl)-6-fluoro-2,4-dichloro-1,8-naphthyridine (1.51 g, 74%) and proceeded to the next reaction without further purification. LC/MS (ESI): m/z=343 [M+H]+.

### Step 4: 7-(8-fluoronaphthyl)-6-fluoro-2-(((S)-1-methylpyrrolidin-2-yl)methoxy)-4-chloro-1,8-naphthyridine

Combine 7-(8-fluoronaphthyl)-6-fluoro-2,4-dichloro-1,8-naphthyridine (1.37 g, 4 mmol), N-methyl-L-prolinol (0.88 g, 4.4 mmol), potassium carbonate (0.88 g, 6.4 mmol) catalytic potassium iodide and DMF (80 mL) were mixed, heated to 120° C., and the reaction was stirred for 4 hours. Cool to room temperature and evaporate under reduced pressure to obtain a yellow solid 7-(8-fluoronaphthyl)-6-fluoro-2-(((S)-1-methylpyrrolidin-2-yl)methoxy)-4-Chloro-1,8-naphthyridine (1.09 g, 62%), LC/MS (ESI): m/z=441 [M+H]+.

### Step 5: 7-(8-fluoronaphthyl)-6-fluoro-2-(((S)-1-methylpyrrolidin-2-yl)methoxy)-4-(((R)-Preparation of 4-boc-2-methylpiperazine)-1-yl)-1,8-naphthyridine

(R)-4-Boc-2-methylpiperazine (0.44 g, 2.2 mmol), 7-(2-fluoro-6-methoxyphenyl)-6-fluoro-2-(((S)-1-Methylpyrrolidin-2-yl)methoxy)-4-chloro-1,8-naphthyridine (0.882 g, 2 mmol), cesium carbonate (1.3 g, 4 mmol) and BINAP (0.124 g, 0.2 mmol) Dissolved in 1.4-dioxane (25 mL), the mixture was degassed by bubbling nitrogen for 5 min. To the reaction was added tris(dibenzylideneacetone)dipalladium (0.1 g, 0.11 mmol) and the reaction mixture was stirred at reflux for 24 hours. After completion of the reaction, the reaction mixture was diluted with ethyl acetate (750 mL), washed with water (100 mL), washed with brine (10 mL), and dried over anhydrous sodium sulfate. Concentration under reduced pressure gave the crude product as a brown solid, which was passed through silica gel containing ethyl acetate (900 mL) to remove any inorganics. It was then recrystallized from acetonitrile to give 7-(8-fluoronaphthyl)-6-fluoro-2-(((S)-1-methylpyrrolidin-2-yl)methoxy)-4-((((R)-4-boc-2-methylpiperazin)-1-yl)-1,8-naphthyridine (1.09 g, 82%). LC/MS (ESI): m/z=604.2 [M+H]+.

### Step 6: 7-(8-fluoronaphthyl)-6-fluoro-2-(((S)-1-methylpyrrolidin-2-yl)methoxy)-4-(((R)2-methylpiperazine)-1-yl)-1,8-naphthyridine

7-(8-Fluoronaphthyl)-6-fluoro-2-(((S)-1-methylpyrrolidin-2-yl)methoxy)-4-(((R)-4-boc-2-Methylpiperazin)-1-yl)-1,8-naphthyridine (302 mg, 0.5 mmol), 2 ml of ethyl acetate, 4 ml of 1 N HCl in 1,4-dioxane. After stirring at room temperature for 2 hours, the reaction solution was neutralized with IN sodium hydroxide solution and extracted with ethyl acetate. The obtained organic phase was washed with saturated sodium bicarbonate and saturated brine, dried over anhydrous sodium sulfate, and the organic phase was evaporated to dryness under reduced pressure. 7-(8-Fluoronaphthyl)-6-fluoro-4-(((R)-2-methylpiperazine)-1-yl)-2-(((S)-1-methylpyrrolidine was obtained -2-yl)methoxy)pyridine[2,3-d]lopyrimidine 25e (183 mg, 73% yield) was used directly in the next step. LC/MS (ESI): m/z=504.2 [M+H]+.

### Step 7: 7-(8-Fluoronaphthyl)-4-(4-acryloyl-2-methylpiperazine)-1-yl)-2-(((S)-1-methylpyrrolidine-2-yl)methoxy)-1,8-naphthyridine

Add 7-(8-fluoronaphthyl)-6-fluoro-2-(((S)-1-methylpyrrolidin-2-yl)methoxy)-4-((((R) 2-Methylpiperazine)-1-yl)-1,8-naphthyridine (60 mg, 0.135 mmol), triethylamine (20.4 mg, 0.2 mmol), 4 ml of tetrahydrofuran, after cooling in an ice-water bath, slowly add acryloyl chloride (18 mg, 0.2 mmol) in 0.5 ml of tetrahydrofuran. After the addition was complete, stirring was continued for 4 hours. The reaction solution was quenched with methanol and evaporated to dryness under reduced pressure. The residue was purified by column chromatography to give 7-(8-fluoronaphthyl)-4-(((R)-4-acryloyl-2-methylpiperazine)-1-yl)-2-((((S)-1-Methylpyrrolidin-2-yl)methoxy)-1,8-naphthyridine (31 mg, 41% yield) was a yellow solid. LC/MS(ESI): m/z=558.2 [M+H]+.

### EXAMPLE 6

7-(8-Fluoronaphthyl)-6-fluoro-4-WS)-4-(2-fluoroacryloyl)-3-carbonitrileethylpiperazine)-1-yl)-2-((((S)-1-methylpyrrolidin-2-yl)methoxy)-1,8-naphthyridine (Compound 6)

Compound 6 was obtained in a similar manner to Example 5 (40 mg, 49% yield). LC/MS (ESI): m/z=601.2 [M+H]+.

### EXAMPLE 7

7-(8-Fluoronaphthyl)-6-fluoro-4-(((R)-4-acryloyl-2-methylpiperazine)-1-yl)-2-(((S)-1-methyl) pyrrolidin-2-yl)methoxy)-1,8-naphthyridine (compound 7)

Compound 7 (29 mg, 38% yield) was obtained in a similar manner to Example 5. LC/MS (ESI): m/z=558.2 [M+H]+.

### EXAMPLE 8

7-(8-Fluoronaphthyl)-6-fluoro-4-(((R)-4-(2-fluoroacryloyl)-2-methylpiperazine)-1-yl)-2-((((S)-1-methylpyrrolidin-2-yl)methoxy)-1,8-naphthyridine (Compound 8)

Compound 8 was obtained in a similar manner to Example 5 (26 mg, 33% yield). LC/MS (ESI): m/z=576.2 [M+H]+.

### EXAMPLE 9

7-(8-Fluoronaphthyl)-6-fluoro-4-(((S)-4-(2-acryloyl)-3-carbonitrileethylpiperazine)-1-yl)-2-((((S)-1-(methyl-d3)pyrrolidin-2-yl)methoxy)pyridine[2,3-d]lopyrimidine (Compound 9)

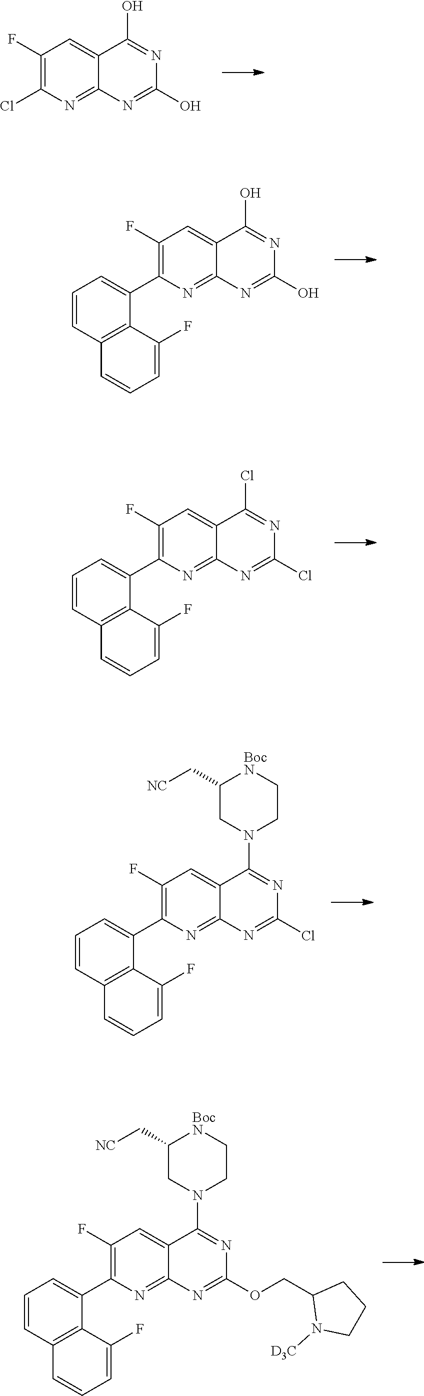 [SMILES: Oc1nc(O)c2cc(F)c(Cl)nc2n1 ; Oc1nc(O)c2cc(F)c(-c3cccc4cccc(F)c34)nc2n1 ; Fc1cc2c(Cl)nc(Cl)nc2nc1-c1cccc2cccc(F)c12 ; CC(C)(C)OC(=O)N1CCN(c2nc(Cl)nc3nc(-c4cccc5cccc(F)c45)c(F)cc23)C[C@@H]1CC#N ; [2H]C([2H])([2H])N1CCCC1COc1nc(N2CCN(C(=O)OC(C)(C)C)[C@@H](CC#N)C2)c2cc(F)c(-c3cccc4cccc(F)c34)nc2n1]

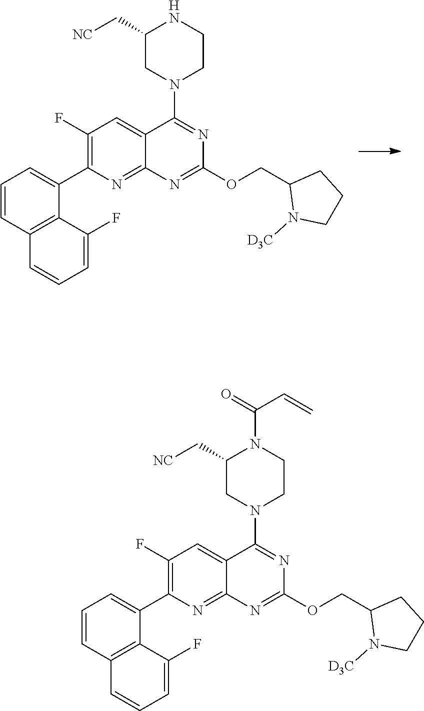 [SMILES: [2H]C([2H])([2H])N1CCCC1COc1nc(N2CCN[C@@H](CC#N)C2)c2cc(F)c(-c3cccc4cccc(F)c34)nc2n1 ; [2H]C([2H])([2H])N1CCCC1COc1nc(N2CCN(C(=O)C=C)[C@@H](CC#N)C2)c2cc(F)c(-c3cccc4cccc(F)c34)nc2n1]

### Step 1: 7-(8-fluoronaphthyl)-6-fluoro-4-(((S)-4-boc-3-carbonitrileethylpiperazine)-1-yl)-2-(((S)-1-(methyl-d3)pyrrolidin-2-yl)methoxy)pyridine[2,3-d]lopyrimidine

Intermediate 7-(2-fluoro-6-methoxyphenyl)-6-fluoro-2,4-dichloropyrido [2,3-d]pyrimidine (0.55 g, 1 mmol), N-(Methyl-d3)-L-prolinol (0.13 g, 1.1 mmol), potassium carbonate (0.28 g, 2 mmol) catalytic potassium iodide and DMF (20 mL) were mixed, heated to 120° C., and stirred for 4 hours. Cool to room temperature and evaporate under reduced pressure to obtain a yellow solid 7-(8-fluoronaphthyl)-6-fluoro-4-(((S)-4-boc-3-carbonitrileethylpiperazine)-1-yl)-2-(((S)-1-(methyl-d3)pyrrolidin-2-yl)methoxy)pyridine[2,3-d]lopyrimidine (0.398 g, 63%), LC/MS (ESI): m/z=633 [M+H]+.

### Step 2: 7-(8-fluoronaphthyl)-6-fluoro-4-(((S)-3-carbonitrile ethylpiperazine)-1-yl)-2-(((S)-1-(methyl-d3)pyrrolidin-2-yl)methoxy)pyridine[2,3-d]lopyrimidine

Add 7-(8-fluoronaphthyl)-6-fluoro-4-(((S)-4-boc-3-carbonitrileethylpiperazine)-1-yl)-2-(((S)-1-(methyl-d3)pyrrolidin-2-yl)methoxy)pyridine[2,3-d]lopyrimidine (0.317 g, 0.5 mmol), 2 ml ethyl acetate, 1 N HCl in 1,4-Dioxane solution 4 ml. After stirring at room temperature for 2 hours, the reaction solution was neutralized with 1N sodium hydroxide solution and extracted with ethyl acetate. The obtained organic phase was washed with saturated sodium bicarbonate and saturated brine, dried over anhydrous sodium sulfate, and the organic phase was evaporated to dryness under reduced pressure. The compound 7-(8-fluoronaphthyl)-6-fluoro-4-(((S)-3-carbonitrileethylpiperazine)-1-yl)-2-(((S)-1-(methyl) was obtained yl-d3)pyrrolidin-2-yl)methoxy)pyridine[2,3-d]lopyrimidine (0.191 g, 72% yield) was used directly in the next step. LC/MS (ESI): m/z=533.2 [M+H]+.

### Step 3: 7-(8-fluoronaphthyl)-6-fluoro-4-(((S)-4-(2-acryloyl)-3-carbonitrile ethylpiperazine)-1-yl)-2-(((S)-1-(methyl-d3)pyrrolidin-2-yl)methoxy)pyridine[2,3-d]lopyrimidine

Add 7-(8-fluoronaphthyl)-6-fluoro-4-(((S)-3-carbonitrileethylpiperazine)-1-yl)-2-(((S)-1 to the reaction flask-(Methyl-d3)pyrrolidin-2-yl)methoxy)pyridine[2,3-d]lopyrimidine (60 mg, 0.135 mmol), triethylamine (20.4 mg, 0.2 mmol), 4 ml tetrahydrofuran, ice After cooling in a water bath, a solution of 2-acryloyl chloride (20 mg, 0.2 mmol) in 0.5 ml of tetrahydrofuran was slowly added dropwise. After the addition was complete, stirring was continued for 4 hours. The reaction solution was quenched with methanol and evaporated to dryness under reduced pressure. The residue was purified by column chromatography to give 7-(8-fluoronaphthyl)-6-fluoro-4-(((S)-4-(2-acryloyl)-3-carbonitrileethylpiperazine)-1-yl)-2-(((S)-1-(methyl-d3)pyrrolidin-2-yl)methoxy)pyridine[2,3-d]lopyrimidine 9 (34 mg, 43% yield) For the yellow solid. LC/MS(ESI): m/z=587.3 [M+H]+.

### EXAMPLE 10

7-(8-Fluoronaphthyl)-6-fluoro-4-(((S)-4-(2-acryloyl)-2-methylpiperazine)-1-yl)-2-((((S)-1-(methyl-d3)pyrrolidin-2-yl)methoxy)pyridine[2,3-d]lopyrimidine (Compound 10)

Compound 10 (42 mg, 56% yield) was obtained in a similar manner to Example 5. LC/MS (ESI): m/z=562.3 [M+H]F.

### EXAMPLE 11

7-(2-Fluoro-6-hydroxyphenyl)-6-fluoro-4-(((S)-4-(2-fluoroacryloyl)-3-carbonitrileethylpiperazine)-1-yl)-2-(((S)-1-(methyl-d3)pyrrolidin-2-yl)methoxy)pyridine[2,3-d]lopyrimidine (Compound 11)

Compound 11 was obtained in a similar manner to Example 1 (30 mg, 41% yield). LC/MS (ESI): m/z=546.2 [M+H]F.

### EXAMPLE 12

7-(2-Fluoro-6-hydroxyphenyl)-6-fluoro-4-(((S)-4-(2-acryloyl)-2-methylpiperazine)-1-yl)-2-(((S)-1-(methyl-d3)pyrrolidin-2-yl)methoxy)pyridine[2,3-d]lopyrimidine (Compound 12)

Compound 12 was obtained in a similar manner to Example 1 (42 mg, 59% yield). LC/MS (ESI): m/z=529.2 [M+H]+.

### EXAMPLE 13

7-(8-Fluoronaphthalene)-4-(((R)-4-Acryloyl-2-methylpiperazine)-1-yl)-2-(((1-(pyrrolidin-1-yl)methyl cyclopropan-1-yl)methoxy)pyridine[2,3-d]lopyrimidine (Compound 13)

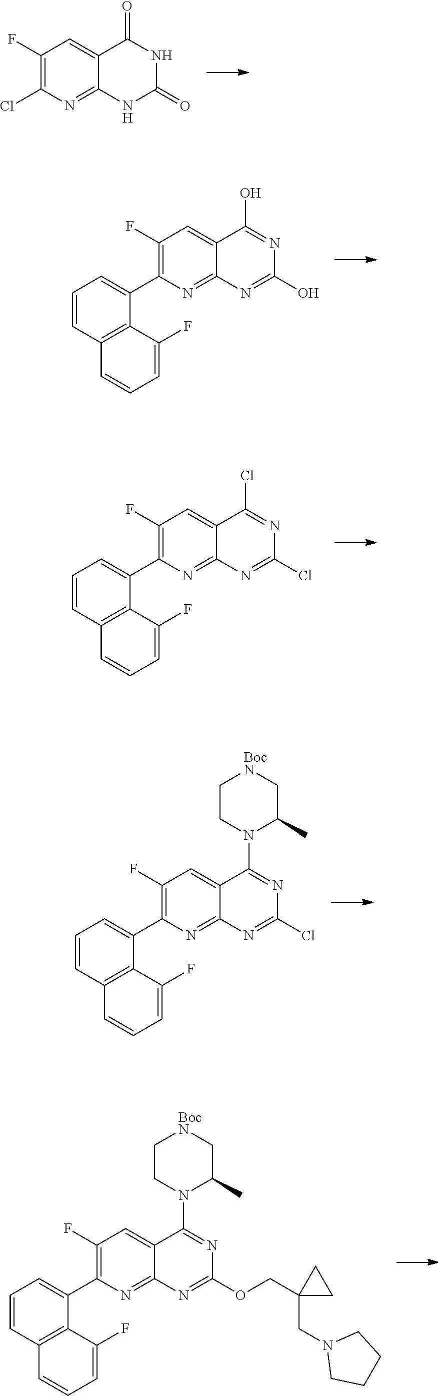 [SMILES: O=c1[nH]c(=O)c2cc(F)c(Cl)nc2[nH]1 ; Oc1nc(O)c2cc(F)c(-c3cccc4cccc(F)c34)nc2n1 ; Fc1cc2c(Cl)nc(Cl)nc2nc1-c1cccc2cccc(F)c12 ; C[C@@H]1CN(C(=O)OC(C)(C)C)CCN1c1nc(Cl)nc2nc(-c3cccc4cccc(F)c34)c(F)cc12 ; C[C@@H]1CN(C(=O)OC(C)(C)C)CCN1c1nc(OCC2(CN3CCCC3)CC2)nc2nc(-c3cccc4cccc(F)c34)c(F)cc12]

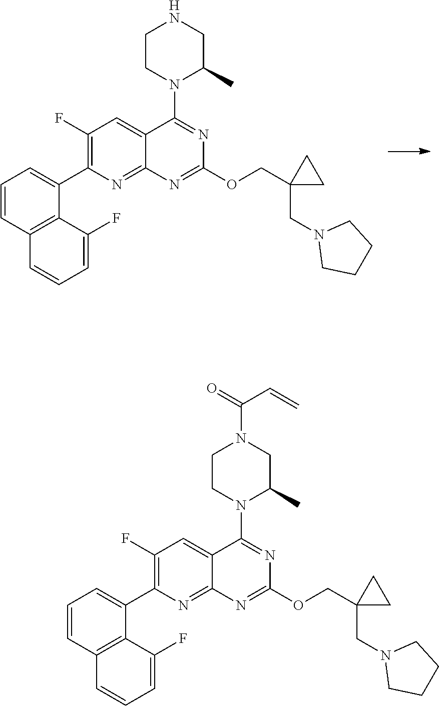 [SMILES: C[C@@H]1CNCCN1c1nc(OCC2(CN3CCCC3)CC2)nc2nc(-c3cccc4cccc(F)c34)c(F)cc12 ; C=CC(=O)N1CCN(c2nc(OCC3(CN4CCCC4)CC3)nc3nc(-c4cccc5cccc(F)c45)c(F)cc23)[C@H](C)C1]

### Step 1: 7-(8-fluoronaphthyl)-6-fluoro-pyrido [2,3-d]pyrimidine-2,4(1H,3H)-dione

6-Fluoro-7-chloropyrido[2,3-d]pyrimidine-2,4(1H,3H)-dione(2.15 g, 0.01 mol), 8-fluoronaphthaleneboronic acid (1.9 g, 0.01 mol)), tris(dibenzylideneacetone)dipalladium (0.8 g, 0.88 mmol), cesium carbonate, 1,4-dioxane (100 mL) and water (20 mL) were mixed, then heated to 120° C. under reflux, stirred The reaction was carried out for 16 hours. The reaction was cooled to room temperature and stirred overnight to give a pale yellow precipitate. The reaction mixture was diluted with water (2 mL) and the solid was collected by filtration. The crude product was slurried with methanol (10 mL) to give 7-(8-fluoronaphthyl)-6-fluoro-pyrido[2,3-d]pyrimidine-2,4(1H,3H)-dione as a beige solid (2.48 g, 76%) and proceeded to the next reaction without further purification.

LC/MS(ESI): m/z=326 [M+H]+.

### Step 2: 7-(8-fluoronaphthyl)-6-fluoro-2,4-dichloropyrido [2,3-d]pyrimidine

7-(8-Fluoronaphthyl)-6-fluoro-pyrido[2,3-d]pyrimidine-2,4(1H,3H)-dione (2.28 g 7 mmol) was dissolved in POCl₃ (30 mL) , add a small amount of N,N-xylene amine, heat under reflux and stir the reaction for 10 h. Then poured into ice water to quench, filtered to obtain solid product, washed with water, and dried to obtain crude yellow solid 7-(8-fluoronaphthyl)-6-fluoro-2,4-dichloropyrido [2,3-d]pyrimidine (1.87 g, 74%) and proceeded to the next reaction without further purification. LC/MS (ESI): m/z=363 [M+H]+.

### Step 3: 7-(8-fluoronaphthyl)-6-fluoro-4-(((R)-4-boc-2-methylpiperazine)-1-yl)-2-chloropyrido [2,3-d]pyrimidine

7-(8-Fluoronaphthyl)-6-fluoro-2,4-dichloropyrido[2,3-d]pyrimidine (181 mg, 0.5 mmol), (R)-4-Boc-2-methyl piperazine (110 mg, 0.55 mmol), potassium carbonate (103 mg, 0.75 mmol) catalytic potassium iodide and DMF (15 mL) were mixed, heated to 120° C., and the reaction was stirred for 4 hours. Cool to room temperature and evaporate under reduced pressure to obtain a yellow solid 7-(8-fluoronaphthyl)-6-fluoro-4-(((R)-4-boc-2-methylpiperazine)-1-yl)-2-Chloropyrido[2,3-d]pyrimidine (200 mg, 76%), LC/MS (ESI): m/z=527 [M+H]+.

### Step 4: 7-(8-fluoronaphthyl)-6-fluoro-4-(((R)-4-boc-2-methylpiperazine)-1-yl)-2-(((1-(pyrrolidin-1-yl)methylcyclopropan-1-yl)methoxy)pyrido[2,3-d]pyrimidine

7-(8-Fluoronaphthyl)-6-fluoro-4-(((R)-4-boc-2-methylpiperazine)-1-yl)-2-chloropyrido[2,3-d] Pyrimidine (174 mg, 0.3 mmol), 0.3 mmol), 1-(1-pyrrolidinylmethyl)cyclopropane-1-methanol (51 mg, 0.33 mmol), potassium carbonate (62 mg, 0.45 mmol) catalytic potassium iodide and DMF (10 mL) Mix, heat to 120° C., and stir the reaction for 4 hours. Cooled to room temperature, evaporated under reduced pressure, and column chromatography gave a yellow solid 7-(8-fluoronaphthyl)-6-fluoro-4-(((R)-4-boc-2-methylpiperazine)-1-yl)-2-(((1-(pyrrolidin-1-yl)methylcyclopropan-1-yl)methoxy)pyrido[2,3-d]pyrimidine (132 mg, 68%). LC/MS (ESI): m/z=645 [M+H]+.

### Step 5: 7-(8-fluoronaphthalene)-4-(((R)-2-methylpiperazine)-1-yl)-2-(((1-(pyrrolidin-1-yl)methylcyclopropan-1-yl)methoxy)pyridine[2,3-d]lopyrimidine

Add the intermediate 7-(8-fluoronaphthyl)-6-fluoro-4-(((R)-4-boc-2-methylpiperazine)-1-yl)-2-(((1-(pyrrolidin-1-yl)methylcyclopropan-1-yl)methoxy)pyrido[2,3-d]pyrimidine (132 mg, 0.2 mmol), 2 ml ethyl acetate, 1 N HCl 4 ml of the 1,4-dioxane solution of the solution. Stir at room temperature for 2 hours, neutralize the reaction solution with 1N sodium hydroxide solution, and extract with ethyl acetate. The obtained organic phase is washed with saturated sodium bicarbonate and saturated brine. Dry over sodium sulfate, and evaporate the organic phase to dryness under reduced pressure. Compound 7-(8-fluoronaphthalene)-4-(((R)-2-methylpiperazine)-1-yl)-2-((((1)-(pyrrolidin-1-yl)methylcyclopropan-1-yl)methoxy)pyridine[2,3-d]lopyrimidine (85 mg, 78% yield) was used directly in the next step. LC/MS (ESI): m/z=545 [M+H]+.

### Step 6: 7-(8-fluoronaphthalene)-4-(((R)-4-acryloyl-2-methylpiperazine)-1-yl)-2-(((1-(pyrrolidine-1-yl)methylcyclopropan-1-yl)methoxy)pyridine[2,3-d]lopyrimidine

Add 7-(8-fluoronaphthyl)-6-fluoro-4-(((R)-2-trifluoromethylpiperazine)-1-yl)-2-chloropyrido[2, 3-d]pyrimidine (76 mg, 0.135 mmol), triethylamine (20.4 mg, 0.2 mmol), 4 ml tetrahydrofuran, after cooling in an ice-water bath, a solution of acryloyl chloride (18 mg, 0.2 mmol) in 0.5 ml tetrahydrofuran was slowly added dropwise. After the addition was complete, stirring was continued for 4 hours. The reaction solution was quenched with methanol and evaporated to dryness under reduced pressure. The residue was purified by column chromatography to give compound 13 (35 mg, 43% yield) as a yellow solid. LC/MS (ESI): m/z=599.3 [M+H]+.

### EXAMPLE 14

7-(8-Chloronaphthalene)-4-(((R)-4-Acryloyl-2-methylpiperazine)-1-yl)-2-(((1-(pyrrolidin-1-yl)methancycloprop an-1-yl)methoxy)pyridine[2,3-d]lopyrimidine (Compound 14)

Compound 14 was obtained in a similar manner to Example 13(48 mg, 58% yield). LC/MS (ESI): m/z=615.3 [M+H]+.

### EXAMPLE 15

7-(8-Fluoronaphthalene)-4-(((R)-4-Acryloyl-3-carbonitrileethylpiperazine)-1-y1)-2-(((1-(pyrrolidin-1-yl)methylcyclopropan-1-yl)methoxy)pyridine[2,3-d]lopyrimidine (Compound 15)

Compound 15 was obtained in a similar manner to Example 13 (47 mg, 56% yield). LC/MS (ESI): m/z=624.3 [M+H]+.

### EXAMPLE 16

7-(8-Chloronaphthalene)-4-(((R)-4-Acryloyl-3-carbonitrileethylpiperazine)-1-yl)-2-(((1-(pyrrolidin-1-yl)methylcyclopropan-1-yl)methoxy)pyridine[2,3-d]lopyrimidine (Compound 16)

Compound 16 was obtained in a similar manner to Example 13 (53 mg, 61% yield). LC/MS (ESI): m/z=640.3 [M+H]+.

### EXAMPLE 17

7-(8-Methylnaphthalene)-4-(((R)-4-Acryloyl-2-methylpiperazine)-1-yl)-2-(((1-(pyrrolidin-1-yl)methylcyclopropan-1-yl)methoxy)pyridine[2,3-d]lopyrimidine (Compound 17)

Compound 17 was obtained in a similar manner to Example 13 (46 mg, 57% yield). LLC/MS (ESI): m/z=595.3 [M+H]+.

### EXAMPLE 18

7-(8-Methylnaphthalene)-4-(((R)-4-Acryloyl-3-carbonitrileethylpiperazine)-1-yl)-2-(((1-(pyrrolidin-1-yl))methylcyclopropan-1-yl)methoxy)pyridine[2,3-d]lopyrimidine (Compound 18)

Compound 18 was obtained in a similar manner to Example 13 (46 mg, 55% yield). LC/MS (ESI): m/z=620.3 [M+H]+.

### EXAMPLE 19

7-(8-Methylnaphthalene)-4-(((R)-4-Acryloyl-3-carbonitrileethylpiperazine)-1-yl)-24(1-(N,N-Dimethyl)amino)methylcyclopropan-1-yl)methoxy)pyridine[2,3-d]lopyrimidine (Compound 19)

Compound 19 was obtained in a similar manner to Example 13 (46 mg, 58% yield). LC/MS (ESI): m/z=594.3 [M+H]+.

### EXAMPLE 20

7-(8-Chloronaphthalene)-4-(((R)-4-Acryloyl-3-carbonitrileethylpiperazine)-1-yl)-24(1-(N,N-dimethylaminyl)methylcyclopropan-1-yl)methoxy)pyridine[2,3-d]lopyrimidine (Compound 20)

Compound 20 was obtained in a similar manner to Example 13 (51 mg, 62% yield). LC/MS (ESI): m/z=614.2 [M+H]+.

### EXAMPLE 21

7-(8-Fluoronaphthalene)-4-(((R)-4-Acryloyl-3-carbonitrileethylpiperazine)-1-yl)-24(1-(N,N-dimethylamineyl)methylcyclopropan-1-yl)methoxy)pyridine[2,3-d]lopyrimidine (Compound 21)

Compound 21 was obtained in a similar manner to Example 13 (52 mg, 64% yield). LC/MS (ESI): m/z=599.3 [M+H]+.

### EXAMPLE 22

7-(8-Methylnaphthalene)-4-(((R)-4-Acryloyl-2-methylpiperazine)-1-yl)-24(1-(N,N-dimethylamineyl)methylcyclopropan-1-yl)methoxy)pyridine[2,3-d]lopyrimidine (Compound 22)

Compound 22 was obtained in a similar manner to Example 13 (44 mg, 57% yield). LC/MS (ESI): m/z=569.3 [M+H]+.

### EXAMPLE 23

7-(8-Chloronaphthalene)-4-(((R)-4-Acryloyl-2-methylpiperazine)-1-yl)-24(1-(N,N-dimethylamino)) methylcyclopropan-1-yl)methoxy)pyridine[2,3-d]lopyrimidine (Compound 23)

Compound 23 was obtained in a similar manner to Example 13(46 mg, 58% yield). LC/MS (ESI): m/z=589.2 [M+H]+.

### EXAMPLE 24

7-(8-Fluoronaphthalene)-4-(((R)-4-Acryloyl-2-methylpiperazine)-1-yl)-24(1-(N,N-dimethylamino)) methylcyclopropan-1-yl)methoxy)pyridine[2,3-d]lopyrimidine (Compound 24)

Compound 24 was obtained in a similar manner to Example 13 (49 mg, 64% yield). LC/MS (ESI): m/z=573.3 [M+H]+.

### EXAMPLE 25

7-(8-Fluoronaphthyl)-6-fluoro-4-(((R)-4-acryloyl-3-carbonitrileethylpiperazine)-1-yl)-2-(((2R,7aS)-2-fluorotetrahydro-1H-pyrincycl-7a(5H)-yl)methoxy)pyridine[2,3-d]lopyrimidine (Compound 25)

Compound 25 was obtained in a similar manner to Example 13 (49 mg, 58% yield). LC/MS (ESI): m/z=628.3 [M+H]+.

### EXAMPLE 26

7-(8-Fluoronaphthyl)-6-fluoro-4-(((R)-4-acryloyl-2-methylpiperazine)-1-yl)-2-(((2R,7aS)-2-Fluorotetrahydro-1H-pyrincycl-7a(5H)-yl)methoxy)pyridine[2,3-d]lopyrimidine (Compound 26)

Compound 26 was obtained in a similar manner to Example 13(46 mg, 56% yield). LC/MS (ESI): m/z=603.3 [M+H]+.

### EXAMPLE 27

7-(8-Fluoronaphthyl)-6-fluoro-4-(8-acryloyl-3,8-diazabicyclo[3.2.1]octan-3-yl)-2-((((2R,7aS)-2-Fluorotetrahydro-1H-pyrincyclo-7a(5H)-yl)methoxy)pyridine[2,3-d]lopyrimidine (Compound 27)

Compound 27 was obtained in a similar manner to Example 13 (45 mg, 54% yield). LC/MS (ESI): m/z=615.3 [M+H]+.

### EXAMPLE 28

7-(8-Fluoronaphthyl)-6-fluoro-4-(8-acryloyl-3,8-diazabicyclo[3.2.1]octan-3-yl)-2-((((2R,7aS)-2-Fluorotetrahydro-1H-pyrincycline-7a(5H)-yl)methoxy)-1,8-naphthyridine (Compound 28)

Compound 28 was obtained in a similar manner to Example 1 (54 mg, 65% yield). LC/MS (ESI): m/z=614.3 [M+H]+.

### EXAMPLE 29

7-(8-Fluoronaphthyl)-6-fluoro-4-(((S)-4-(2-fluoroacryloyl)-3-carbonitrileethylpiperazine)-1-yl)-2-((((2R,7aS)-2-fluorotetrahydro-1H-pyrinyl-7a(5H)-yl)methoxy)pyridine[2,3-d]lopyrimidine (Compound 29)

Compound 29 was obtained in a similar manner to Example 13 (36 mg, 41% yield). LC/MS (ESI): m/z=646.3 [M+H]+.

### EXAMPLE 30

7-(8-Fluoronaphthyl)-6-fluoro-4-(((R)-4-(2-fluoroacryloyl)-2-methylpiperazine)-1-yl)-2-((((2R,7aS)-2-fluorotetrahydro-1H-pyrincycline-7a(5H)-yl)methoxy)pyridine[2,3-d]lopyrimidine (Compound 29)

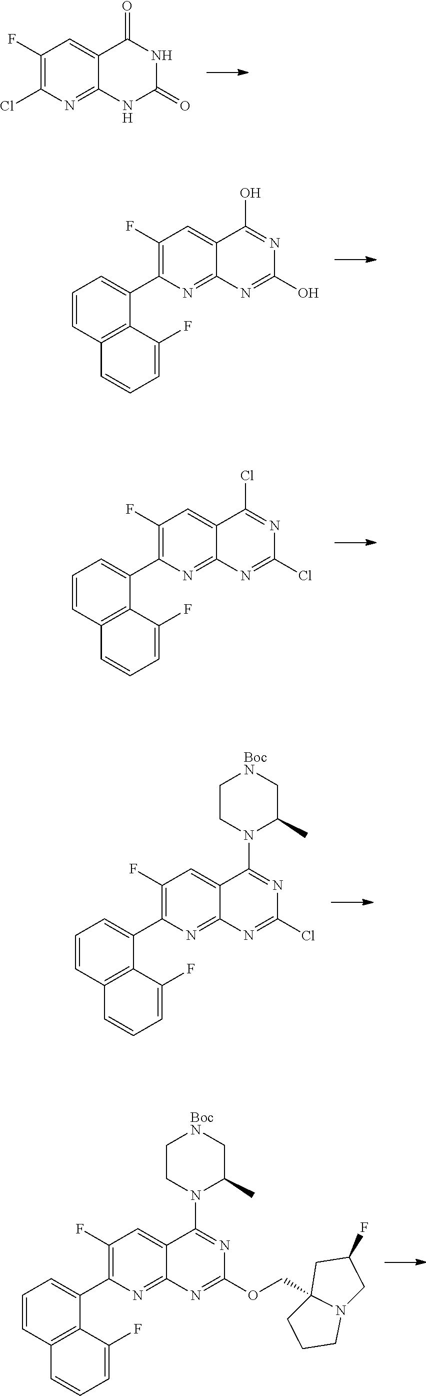 [SMILES: O=c1[nH]c(=O)c2cc(F)c(Cl)nc2[nH]1 ; Oc1nc(O)c2cc(F)c(-c3cccc4cccc(F)c34)nc2n1 ; Fc1cc2c(Cl)nc(Cl)nc2nc1-c1cccc2cccc(F)c12 ; C[C@@H]1CN(C(=O)OC(C)(C)C)CCN1c1nc(Cl)nc2nc(-c3cccc4cccc(F)c34)c(F)cc12 ; C[C@@H]1CN(C(=O)OC(C)(C)C)CCN1c1nc(OC[C@@]23CCCN2C[C@H](F)C3)nc2nc(-c3cccc4cccc(F)c34)c(F)cc12]

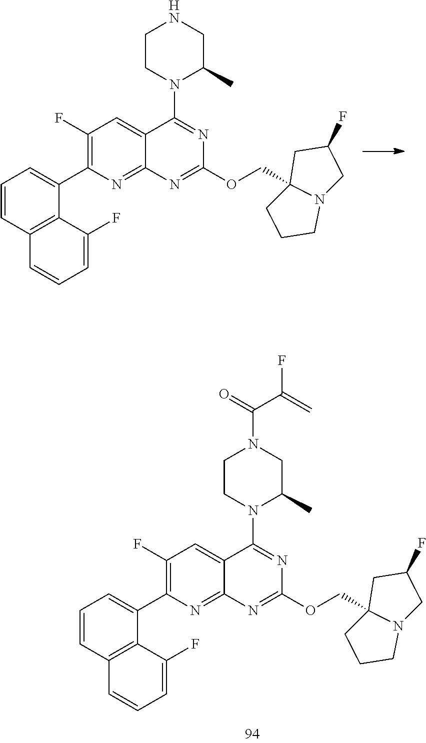 [SMILES: C[C@@H]1CNCCN1c1nc(OC[C@@]23CCCN2C[C@H](F)C3)nc2nc(-c3cccc4cccc(F)c34)c(F)cc12 ; C=C(F)C(=O)N1CCN(c2nc(OC[C@@]34CCCN3C[C@H](F)C4)nc3nc(-c4cccc5cccc(F)c45)c(F)cc23)[C@H](C)C1]

### Step 1: 7-(8-fluoronaphthyl)-6-fluoro-2,4-dichloropyrido [2,3-d]pyrimidine

7-(2-Fluoro-6-methoxyphenyl)-6-fluoro-pyrido [2,3-d]pyrimidine-2,4(1H,3H)-dione (1.95 g 6 mmol) was dissolved in POCl₃ (30 mL), a small amount of N,N-xylene amine was added, and the reaction was stirred under reflux for 10 h. Then poured into ice water to quench, filtered to obtain solid product, washed with water, and dried to obtain crude yellow solid 7-(8-fluoronaphthyl)-6-fluoro-2,4-dichloropyrido [2,3-d]pyrimidine (1.72 g, 79%) and proceeded to the next reaction without further purification. LC/MS(ESI): m/z=363 [M+H]+.

### Step 2: 7-(8-fluoronaphthyl)-6-fluoro -4-(((R)-4-boc -2-methylpiperazine)-1-yl)-2-chloropyrido [2,3-d]pyrimidine

7-(8-Fluoronaphthyl)-6-fluoro-2,4-dichloropyrido[2,3-d]pyrimidine (1.45 g, 4 mmol), (R)-4-Boc-2-methyl Piperazine (0.88 g, 4.4 mmol), potassium carbonate (0.88 g, 6.4 mmol) catalytic potassium iodide and DMF (80 mL) were mixed, heated to 120° C., and the reaction was stirred for 4 hours. Cool to room temperature and evaporate under reduced pressure to obtain a yellow solid 7-(8-fluoronaphthyl)-6-fluoro-4-(((R)-4-boc-2-methylpiperazine)-1-yl)-2-Chloropyrido [2,3-d]pyrimidine (1.56 g, 74%), LC/MS (ESI): m/z=527 [M+H]+.

### Step 3: 7-(8-fluoronaphthyl)-6-fluoro-4-(((R)-4-boc-2-methylpiperazine)-1-yl)-2-(((2R, 7aS)-2-fluorotetrahydro-1H-pyrincycline-7a(5H)-yl)methoxy)pyrido [2,3-d]pyrimidine

7-(8-Fluoronaphthyl)-6-fluoro-4-(((R)-4-boc-2-methylpiperazine)-1-yl)-2-chloropyrido [2,3-d] Pyrimidine (158 mg, 0.3 mmol), (2R,8S)-2-fluorotetrahydro-1H-pyrinyl-7a(5H)-yl)methanol (53 mg, 0.33 mmol), potassium carbonate (62 mg, 0.45 mmol)) A catalytic amount of potassium iodide and DMF (10 mL) were mixed, heated to 120° C., and the reaction was stirred for 4 hours. Cooled to room temperature, evaporated under reduced pressure, and column chromatography gave a yellow solid 7-(8-fluoronaphthyl)-6-fluoro-4-(((R)-4-boc-2-methylpiperazine)-1-yl)-2-(((2R,7aS)-2-fluorotetrahydro-1H-pyrincycline-7a(5H)-yl)methoxy)pyrido[2,3-d]pyrimidine (130 mg, 67%). LC/MS (ESI): m/z=649.3 [M+H]+.

### Step 4: 7-(8-fluoronaphthyl)-6-fluoro-4-(((R)-2-methylpiperazine)-1-yl)-2-(((2R,7aS)-2-fluorotetrahydro-1H-pyrincycl-7a(5H)-yl)methoxy))pyrido[2,3-d]pyrimidine

7-(8-Fluoronaphthyl)-6-fluoro-4-(((R)-4-boc-2-methylpiperazine)-1-yl)-2-(((2R,7aS)-2-Fluorotetrahydro-1H-pyrincycl-7a(5H)-yl)methoxy)pyrido[2,3-d]pyrimidine (117 mg, 0.18 mmol) was dissolved in 1 ml of ethyl acetate and 1N HCl in 1,4-dioxane solution 2 ml. After stirring at room temperature for 2 hours, the reaction solution was neutralized with 1N sodium hydroxide solution and extracted with ethyl acetate. The obtained organic phase was washed with saturated sodium bicarbonate and saturated brine, dried over anhydrous sodium sulfate, and the organic phase was evaporated to dryness under reduced pressure. The compound 7-(8-fluoronaphthyl)-6-fluoro-4-(((R)-2-methylpiperazine)-1-yl)-2-((((2R,7aS)-2-fluorotetrahydro-1H-pyrincycl-7a(5H)-yl)methoxy))pyrido[2,3-d]pyrimidine (82 mg, 84% yield) was used directly in the next step. LC/MS (ESI): m/z=549.3 [M+H]+.

### Step 5: 7-(8-fluoronaphthyl)-6-fluoro-4-(((R)-4-(2-fluoroacryloyl)-2-methylpiperazine)-1-yl)-2-(((2R,7aS)-2-fluorotetrahydro-1H-pyrinyl-7a(5H)-yl)methoxy)pyridine[2,3-d]lopyrimidine

Add 7-(8-fluoronaphthyl)-6-fluoro-4-(((R)-2-methylpiperazine)-1-yl)-2-(((2R,7aS)-2-Fluorotetrahydro-1H-pyrincycl-7a(5H)-yl)methoxy))pyrido[2,3-d]pyrimidine (74 mg, 0.135 mmol), triethylamine (20.4 mg, 0.2 mmol)), 4 ml of tetrahydrofuran, after cooling in an ice-water bath, a solution of 2-fluoroacryloyl chloride (18 mg, 0.2 mmol) in 0.5 ml of tetrahydrofuran was slowly added dropwise. After the addition was complete, stirring was continued for 4 hours. The reaction solution was quenched with methanol and evaporated to dryness under reduced pressure. The residue was purified by column chromatography to give compound 30 (32 mg, 53% yield) as a yellow solid. LC/MS(ESI): m/z=621.3 [M+H]+.

### EXAMPLE 31

7-(8-Fluoronaphthyl)-6-fluoro-4-(8-(2-fluoroacryloyl)-3,8-diazabicyclo[3.2.1]octan-3-yl)-2-(((2R,7aS)-2-fluorotetrahydro-1H-pyrinyl-7a(5H)-yl)methoxy)pyridine[2,3-d]lopyrimidine (Compound 31)

Compound 31 was obtained in a similar manner to Example 30 (50 mg, 59% yield). LC/MS (ESI): m/z=623.3 [M+H]+.

### EXAMPLE 32

7-(3-Hydroxy-8-fluoronaphthyl)-6-fluoro-4-(8-(2-fluoroacryloyl)-3,8-diazabicyclo[3.2.1]octan-3-yl)-2-(((2R,7aS)-2-fluorotetrahydro-1H-pyrincycl-7a(5H)-yl)methoxy)pyridine[2,3-d]lopyrimidine (Compound 32)

Compound 32 was obtained in a similar manner to Example 30 (47 mg, 54% yield). LC/MS (ESI): m/z=649.3 [M+H]+.

### EXAMPLE 33

7-(3-Hydroxy-8-fluoronaphthyl)-6-fluoro-4-(((S)-4-(2-fluoroacryloyl)-3-carbonitrileethylpiperazine)-1-yl)-2-(((2R,7aS)-2-fluorotetrahydro-1H-pyrincycl-7a(5H)-yl)methoxy)pyridine[2,3-d]lopyrimidine (Compound 33)

Compound 33 (32 mg, 36% yield) was obtained in a similar manner to Example 30. LC/MS (ESI): m/z=662.3 [M+H]+.

### EXAMPLE 34

7-(3-Hydroxy-8-fluoronaphthyl)-6-fluoro-4-(((R)-4-(2-fluoroacryloyl)-2-methylpiperazine)-1-yl)-2-(((2R,7aS)-2-fluorotetrahydro-1H-pyrincyclin-7a(5H)-yl)methoxy)pyridine[2,3-d]lopyrimidine (Compound 34)

Compound 34 was obtained in a similar manner to Example 30 (33 mg, 39% yield). LC/MS (ESI): m/z=637.3 [M+H]+.

### EXAMPLE 35

7-(3-Hydroxy-8-fluoronaphthyl)-6-fluoro-4-(((S)-4-acryloyl-3-carbonitrileethylpiperazine)-1-yl)-2-(((2R,7aS)-2-fluorotetrahydro-1H-pyrinyl-7a(5H)-yl)methoxy)pyridine[2,3-d]lopyrimidine (Compound 35)

Compound 35 was obtained in a similar manner to Example 30 (28 mg, 32% yield). LC/MS (ESI): m/z=644.3 [M+H]+.

### EXAMPLE 36

7-(3-Hydroxy-8-fluoronaphthyl)-6-fluoro-4-(((R)-4-acryloyl-2-methylpiperazine)-1-yl)-2-(((2R,7aS)-2-fluorotetrahydro-1H-pyrincyclo-7a(5H)-yl)methoxy)pyridine[2,3-d]lopyrimidine (Compound 36)

Compound 36 was obtained in a similar manner to Example 30 (46 mg, 55% yield). LC/MS (ESI): m/z=619.3 [M+H]+.

### EXAMPLE 37

6-Chloro-7-(8-fluoronaphthyl)-8-fluoro-4-(((R)-4-acryloyl-2-methylpiperazine)-1-yl)-2-(((2R,7aS)-2-fluorotetrahydro-1H-pyrincycline-7a(5H)-yl)methoxy)quinazoline (compound 37)

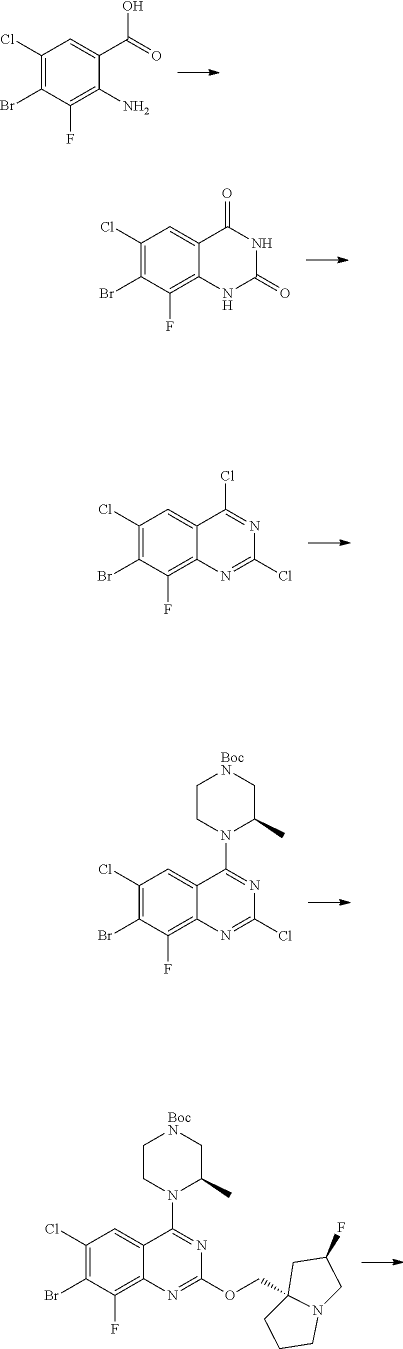 [SMILES: Nc1c(C(=O)O)cc(Cl)c(Br)c1F ; O=c1[nH]c(=O)c2cc(Cl)c(Br)c(F)c2[nH]1 ; Fc1c(Br)c(Cl)cc2c(Cl)nc(Cl)nc12 ; C[C@@H]1CN(C(=O)OC(C)(C)C)CCN1c1nc(Cl)nc2c(F)c(Br)c(Cl)cc12 ; C[C@@H]1CN(C(=O)OC(C)(C)C)CCN1c1nc(OC[C@@]23CCCN2C[C@H](F)C3)nc2c(F)c(Br)c(Cl)cc12]

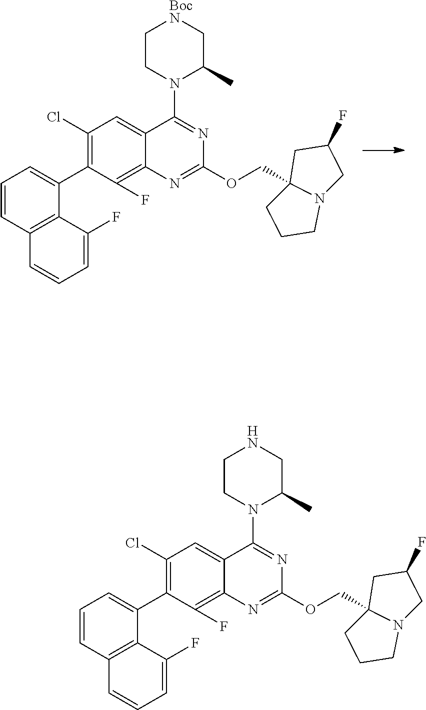 [SMILES: C[C@@H]1CN(C(=O)OC(C)(C)C)CCN1c1nc(OC[C@@]23CCCN2C[C@H](F)C3)nc2c(F)c(-c3cccc4cccc(F)c34)c(Cl)cc12 ; C[C@@H]1CNCCN1c1nc(OC[C@@]23CCCN2C[C@H](F)C3)nc2c(F)c(-c3cccc4cccc(F)c34)c(Cl)cc12]

### Step 1: 7-bromo-8-fluoro-6-chloro-2,4-quinazolinedione

3-Fluoro-4-bromo-5-chloro-2-aminobenzoic acid (13.4 g, 0.05 mol ) and urea (45 g, 0.75 mol ) were heated to 150° C., stirred for 12 hours, then cooled to 95° C., and then 200 mL of water was added, stirred for half an hour, filtered, slurried with acetic acid, and then dried to obtain as a yellow solid 7-bromo-8-fluoro-6-chloro-2,4-quinazolinedione (12.62 g, 86%). LC/MS(ESI): m/z=294.5 [M+H]+.

### Step 2: 7-bromo-8-fluoro-2,4,6-trichloroquinazoline

7-Bromo-8-fluoro-6-chloro-2,4-quinazolinedione (1.76 g 6 mmol) was dissolved in POCl3 (30 mL), a small amount of N,N-xylidine was added, and the reaction was stirred under reflux for 10 h. Then poured into ice water to quench, filtered to obtain solid product, washed with water, and dried to obtain crude yellow solid 7-bromo-8-fluoro-2,4,6-trichloroquinazoline (1.70 g, 86%), which was carried out without further purification. next reaction. LC/MS(ESI): m/z=331 [M+H]+.

### Step 3: 2,6-dichloro-7-bromo-8-fluoro-4-(((R)-4-boc-2-methylpiperazine)-1-yl))quinazoline

7-Bromo-8-fluoro-2,4,6-trichloroquinazoline (1.32 g, 4 mmol), (R)-4-Boc-2-methylpiperazine (0.88 g, 4.4 mmol), potassium carbonate (0.88 g, 6.4 mmol) catalytic potassium iodide and DMF (80 mL) were mixed, heated to 120° C., and the reaction was stirred for 4 hours. Cooled to room temperature, evaporated under reduced pressure to obtain yellow solid 2,6-dichloro-7-bromo-8-fluoro-4-(((R)-4-boc-2-methylpiperazine)-1-yl)) quinazoline (1.56 g, 79%), LC/MS (ESI): m/z=495 [M-FH]+.

### Step 4: 6-chloro-7-bromo-8-fluoro-4-(((R)-4-boc-2-methylpiperazine)-1-yl))-2-(((2R,7aS)-2-fluorotetrahydro-1H-pyrincycline-7a(5H)-yl)methoxy)quinazoline

2,6-Dichloro-7-bromo-8-fluoro-4-(((R)-4-boc-2-methylpiperazin)-1-yl))quinazoline (148 mg, 0.3 mmol), (2R,8S)-2-fluorotetrahydro-1H-pyrinyl-7a(5H)-yl)methanol (53 mg, 0.33 mmol), potassium carbonate (62 mg, 0.45 mmol) catalytic potassium iodide and DMF (10 mL) Mix, heat to 120° C., and stir the reaction for 4 hours. Cooled to room temperature, evaporated under reduced pressure, and column chromatography gave yellow solid 6-chloro-7-bromo-8-fluoro-4-(((R)-4-boc-2-methylpiperazine)-1-yl))-2-(((2R,7aS)-2-fluorotetrahydro-1H-pyrincyclin-7a(5H)-yl)methoxy)quinazoline (137 mg, 74%). LC/MS (ESI): m/z=618 [M-FH]+.

### Step 5: 6-chloro-7-(8-fluoronaphthyl)-8-fluoro-4-(((R)-4-boc-2-methylpiperazine)-1-yl))-2-(((2R,7aS)-2-fluorotetrahydro-1H-pyrincycline-7a(5H)-yl)methoxy)quinazoline

6-Chloro-7-bromo-8-fluoro-4-(((R)-4-boc-2-methylpiperazine)-1-yl))-2-(((2R,7aS)-2-Fluorotetrahydro-1H-pyrinyl-7a(5H)-yl)methoxy)quinazoline (123 mg, 0.2 mmol), 8-fluoronaphthalene-1-boronic acid (38 mg, 0.2 mmol), tris(di) benzylideneacetone)dipalladium (0.017 g, 0.018 mmol), cesium carbonate, 1,4-dioxane (4 mL) and water (1 mL) were mixed, then heated to 120° C. under reflux, and the reaction was stirred for 16 hours. The reaction was cooled to room temperature and stirred overnight to give a pale yellow precipitate. The reaction mixture was diluted with water (2 mL) and the solid was collected by filtration. Drying gave 6-chloro-7-(8-fluoronaphthyl)-8-fluoro-4-(((R)-4-boc-2-methylpiperazine)-1-yl))-2- as a yellow solid (((2R,7aS)-2-fluorotetrahydro-1H-pyrincycl-7a(5H)-yl)methoxy)quinazoline (119 mg, 87%) was carried to the next reaction without further purification. LC/MS (ESI): m/z=683 [M+H]+.

### Step 6: 6-chloro-7-(8-fluoronaphthyl)-8-fluoro-4-(((R)-2-methylpiperazine)-1-yl))-2-((((2R),7aS)-2-fluorotetrahydro-1H-pyrinyl-7a(5H)-yl)methoxy)quinazoline

6-Chloro-7-(8-fluoronaphthyl)-8-fluoro-4-(((R)-4-boc-2-methylpiperazine)-1-yl))-2-((((2R,7aS)-2-fluorotetrahydro-1H-pyrinyl-7a(5H)- yl)methoxy)quinazoline (116 mg, 0.17 mmol) in 1 ml of ethyl acetate and 1N HCl in 1,4-Dioxane solution 2 ml. After stirring at room temperature for 2 hours, the reaction solution was neutralized with 1N sodium hydroxide solution and extracted with ethyl acetate. The obtained organic phase was washed with saturated sodium bicarbonate and saturated brine, dried over anhydrous sodium sulfate, and the organic phase was evaporated to dryness under reduced pressure. The compound 6-chloro-7-(8-fluoronaphthyl)-8-fluoro-4-(((R)-2-methylpiperazin)-1-yl))-2-((((2R,7aS) was obtained)-2-fluorotetrahydro-1H-pyrincycl-7a(5H)-yl)methoxy)quinazoline (83 mg, 84% yield) was used directly in the next step. LC/MS (ESI): m/z=583 [M+H]+.

### Step 7: 6-chloro-7-(8-fluoronaphthyl)-4-(((R)-4-acryloyl-2-methylpiperazine)-1-yl))-8-fluoro-2-(((2R,7aS)-2-fluorotetrahydro-1H-pyrincycline-7a(5H)-yl)methoxy)quinazoline

Add 6-chloro-7-(8-fluoronaphthyl)-8-fluoro-4-(((R)-2-methylpiperazine)-1-yl))-2-((((2R,7aS)-2-fluorotetrahydro-1H-pyrinyl-7a(5H)-yl)methoxy)quinazoline (79 mg, 0.135 mmol), triethylamine (20.4 mg, 0.2 mmol), 4 ml Tetrahydrofuran, after cooling in an ice-water bath, a solution of 2-fluoroacryloyl chloride (18 mg, 0.2 mmol) in 0.5 ml of tetrahydrofuran was slowly added dropwise. After the addition was complete, stirring was continued for 4 hours. The reaction solution was quenched with methanol and evaporated to dryness under reduced pressure. The residue was purified by column chromatography to give compound 37 (44 mg, yield 51%) as a yellow solid. LC/MS(ESI): m/z=637.2 [M+H]+.

### EXAMPLE 38

6-Chloro-7-(8-fluoronaphthyl)-8-fluoro-4-(((R)-4-(2-fluoroacryloyl)-2-methylpiperazine)-1-yl)-2-(((2R,7aS)-2-fluorotetrahydro-1H-pyrinyl-7a(5H)-yl)methoxy)quinazoline (Compound 38)

Compound 38 was obtained in a similar manner to Example 37 (51 mg, 58% yield). LC/MS (ESI): m/z=654.2 [M+H]+.

### EXAMPLE 39

6-Chloro-7-(8-fluoronaphthyl)-8-fluoro-4-(8-(2-fluoroacryloyl)-3,8-diazabicyclo[3.2.1]octan-3-yl)-2-(((2R,7aS)-2-fluorotetrahydro-1H-pyrincycline-7a(5H)-yl)methoxy)quinazoline (compound 39)

Compound 39 (29 mg, 32% yield) was obtained in a similar manner to Example 37. LC/MS (ESI): m/z=666.2 [M+H]+.

### EXAMPLE 40

6-Chloro-7-(8-fluoronaphthyl)-8-fluoro-4-(((S)-4-acryloyl-3-carbonitrileethylpiperazine)-1-yl)-2-(((2R,7aS)-2-fluorotetrahydro-1H-pyrinyl-7a(5H)-yl)methoxy)quinazoline (compound 40)

Compound 40 was obtained in a similar manner to Example 37 (52 mg, 58% yield). LC/MS (ESI): m/z=661.2 [M+H]+.

### EXAMPLE 41

6-Chloro-7-(8-fluoronaphthyl)-8-fluoro-4-(((S)-4-(2-fluoroacryloyl)-3-carbonitrileethylpiperazine)-1-yl)-2-(((2R,7aS)-2-fluorotetrahydro-1H-pyrincycline-7a(5H)-yl)methoxy)quinazoline (Compound 41)

Compound 41 was obtained in a similar manner to Example 37 (35 mg, 38% yield). LC/MS (ESI): m/z=679.2 [M+H]+.

### EXAMPLE 42

6-Chloro-74(3-hydroxy-8-fluoronaphthyl)-8-fluoro-4-(8-(2-fluoroacryloyl)-3,8-diazabicyclo[3.2.1]octane-3-yl)-2-(((2R,7aS)-2-fluorotetrahydro-1H-pyrincycline-7a(5H)-yl)methoxy)quinazoline (Compound 42)

Compound 42 (33 mg, 36% yield) was obtained in a similar manner to Example 37. LC/MS (ESI): m/z=682.2 [M+H]+.

### EXAMPLE 43

6-Chloro-74(3-hydroxy-8-fluoronaphthyl)-8-fluoro-4-(((S)-4-(2-fluoroacryloyl)-3-carbonitrileethylpiperazine)-1-yl)-2-(((2R,7aS)-2-fluorotetrahydro-1H-pyrincycline-7a(5H)-yl)methoxy)quinazoline (Compound 43)

Compound 43 was obtained in a similar manner to Example 37 (28 mg, 31% yield). LC/MS (ESI): m/z=678.2 [M+H]+.

### EXAMPLE 44

6-Chloro-74(3-hydroxy-8-fluoronaphthyl)-8-fluoro-4-(((R)-4-(2-fluoroacryloyl)-2-methylpiperazine)-1-yl)-2-(((2R,7aS)-2-fluorotetrahydro-1H-pyrincycline-7a(5H)-yl)methoxy)quinazoline (Compound 44)

Compound 44 was obtained in a similar manner to Example 37 (26 mg, 29% yield). LC/MS (ESI): m/z=670.2 [M+H]+.

### EXAMPLE 45

6-Chloro-74(3-hydroxy-8-fluoronaphthyl)-8-fluoro-4-(((S)-4-(2-fluoroacryloyl)-3-carbonitrileethylpiperazine)-1-yl)-2-(((2R,7aS)-2-fluorotetrahydro-1H-pyrincycline-7a(5H)-yl)methoxy)quinazoline (Compound 45)

Compound 45 was obtained in a similar manner to Example 37 (53 mg, 58% yield). LC/MS (ESI): m/z=677.2 [M+H]+.

### EXAMPLE 46

6-Chloro-7-(3-hydroxy-8-fluoronaphthyl)-8-fluoro-4-(((R)-4-acryloyl-2-methylpiperazine)-1-yl)-2-(((2R,7aS)-2-fluorotetrahydro-1H-pyrinyl-7a(5H)-yl)methoxy)quinazoline (Compound 46)

Compound 46 was obtained in a similar manner to Example 37 (56 mg, 64% yield). LC/MS (ESI): m/z=652.2 [M+H]+.

### EXAMPLE 47

7-(8-Fluoronaphthyl)-8-fluoro-4-(((R)-4-acryloyl-2-methylpiperazine)-1-yl)-2-(((2R,7aS)-2-fluorotetrahydro-1H-pyrincyclo-7a(5H)-yl)methoxy)quinazoline (compound 47)

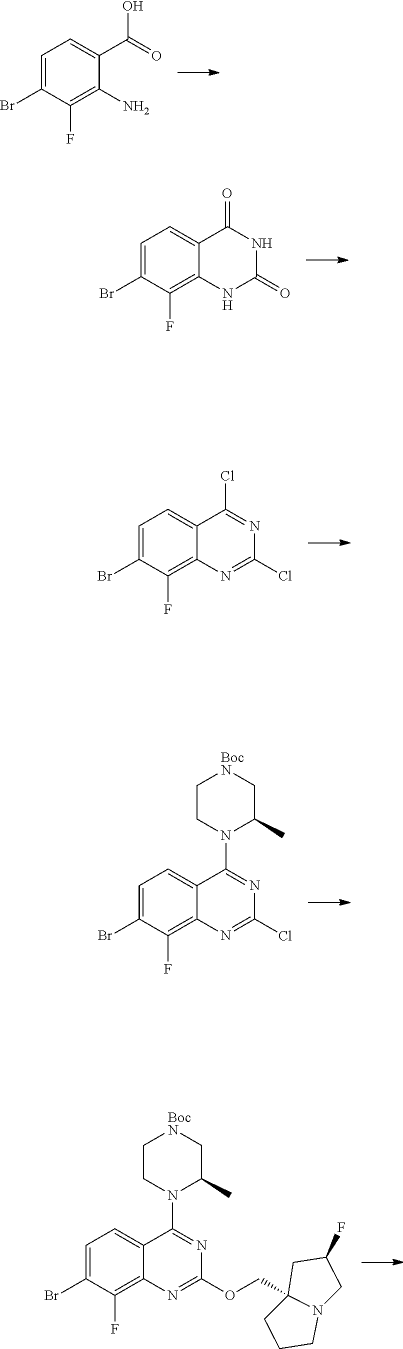 [SMILES: Nc1c(C(=O)O)ccc(Br)c1F ; O=c1[nH]c(=O)c2ccc(Br)c(F)c2[nH]1 ; Fc1c(Br)ccc2c(Cl)nc(Cl)nc12 ; C[C@@H]1CN(C(=O)OC(C)(C)C)CCN1c1nc(Cl)nc2c(F)c(Br)ccc12 ; C[C@@H]1CN(C(=O)OC(C)(C)C)CCN1c1nc(OC[C@@]23CCCN2C[C@H](F)C3)nc2c(F)c(Br)ccc12]

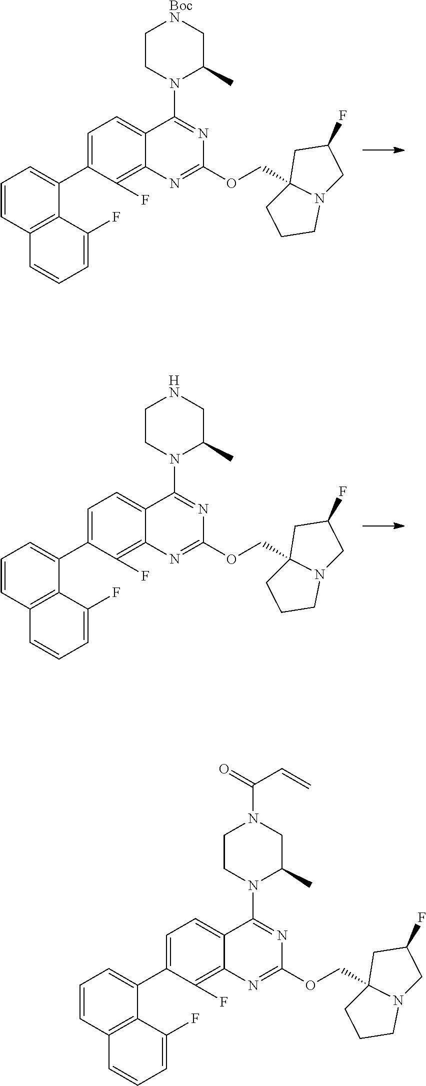 [SMILES: C[C@@H]1CN(C(=O)OC(C)(C)C)CCN1c1nc(OC[C@@]23CCCN2C[C@H](F)C3)nc2c(F)c(-c3cccc4cccc(F)c34)ccc12 ; C[C@@H]1CNCCN1c1nc(OC[C@@]23CCCN2C[C@H](F)C3)nc2c(F)c(-c3cccc4cccc(F)c34)ccc12 ; C=CC(=O)N1CCN(c2nc(OC[C@@]34CCCN3C[C@H](F)C4)nc3c(F)c(-c4cccc5cccc(F)c45)ccc23)[C@H](C)C1]

### Step 1: 7-bromo-8-fluoro-2,4-quinazolinedione

3-Fluoro-4-bromo-2-aminobenzoic acid (11.7 g, 0.05 mol ) and urea (45 g, 0.75 mol ) were heated to 150° C., stirred and reacted for 12 hours, then cooled to 95° C., and then 200 mL of water was added, It was stirred for half an hour, filtered, slurried with acetic acid, and dried to give 7-bromo-8-fluoro-2,4-quinazolinedione (11.88 g, 87%) as a yellow solid. LC/MS(ESI): m/z=274 [M+H]+.

### Step 2: 7-bromo-8-fluoro-2,4-dichloroquinazoline

7-Bromo-8-fluoro-2,4-quinazolinedione (10.92 g 40 mmol) was dissolved in POCl₃ (100 mL), a small amount of N,N-xylidine was added, and the reaction was stirred under reflux for 10 h. Then poured into ice water to quench, filtered to obtain a solid product, washed with water, and dried to obtain a crude yellow solid 7-bromo-8-fluoro-2,4-dichloroquinazoline (9.94 g, 84%), and the next step was carried out without further purification. reaction. LC/MS(ESI): m/z=297 [M+H]+.

### Step 3: 2-chloro-7-bromo-8-fluoro-4-(((R)-4-boc-2-methylpiperazine)-1-yl))quinazoline

7-Bromo-8-fluoro-2,4-dichloroquinazoline (1.18 g, 4 mmol), (R)-4-Boc-2-methylpiperazine (0.88 g, 4.4 mmol), potassium carbonate (0.88 g g, 6.4 mmol) catalytic potassium iodide and DMF (80 mL) were mixed, heated to 120° C., and the reaction was stirred for 4 hours. Cooled to room temperature, evaporated under reduced pressure to obtain yellow solid 2-chloro-7-bromo-8-fluoro-4-(((R)-4-boc-2-methylpiperazine)-1-yl))quinoline oxazoline (1.51 g, 82%), LC/MS (ESI): m/z=460 [M-FH]+.

### Step 4: 6-chloro-7-bromo-8-fluoro-4-(((R)-4-boc-2-methylpiperazine)-1-yl))-2-(((2R,7aS)-2-fluorotetrahydro-1H-pyrincycline-7a(5H)-yl)methoxy)quinazoline

2-Chloro-7-bromo-8-fluoro-4-(((R)-4-boc-2-methylpiperazin)-1-yl))quinazoline (275 mg, 0.6 mmol), (2R ,8S)-2-fluorotetrahydro-1H-pyrinyl-7a(5H)-yl)methanol (106 mg, 0.66 mmol), potassium carbonate (124 mg, 0.90 mmol) catalytic potassium iodide and DMF (20 mL) were mixed, heated To 120° C., the reaction was stirred for 4 hours. Cooled to room temperature, evaporated under reduced pressure, and obtained by column chromatography as a yellow solid 7-bromo-8-fluoro-4-(((R)-4-boc-2-methylpiperazine)-1-yl))-2-(((2R,7aS)-2-fluorotetrahydro-1H-pyrincycl-7a(5H)-yl)methoxy)quinazoline (310 mg, 89%). LC/MS (ESI): m/z=583.2 [M-FH]+.

### Step 5: 7-(8-fluoronaphthyl)-8-fluoro-4-(((R)-4-boc-2-methylpiperazine)-1-yl))-2-(((2R,7aS)-2-fluorotetrahydro-1H-pyrinyl-7a(5H)-yl)methoxy)quinazoline

7-Bromo-8-fluoro-4-(((R)-4-boc-2-methylpiperazine)-1-yl))-2-(((2R,7aS)-2-fluorotetrahydro -1H-Pyridine-7a(5H)-yl)methoxy)quinazoline (175 mg, 0.3 mmol), 8-fluoronaphthalene-1-boronic acid (57 mg, 0.3 mmol), tris(dibenzylideneacetone)) Dipalladium (0.026 g, 0.027 mmol), cesium carbonate, 1,4-dioxane (6 mL) and water (1.5 mL) were mixed, then heated to 120° C. under reflux, and the reaction was stirred for 16 hours. The reaction was cooled to room temperature and stirred overnight to give a pale yellow precipitate. The reaction mixture was diluted with water (2 mL) and the solid was collected by filtration. Drying gave 7-(8-fluoronaphthyl)-8-fluoro-4-(((R)-4-boc-2-methylpiperazine)-1-yl))-2-(((2R) as a yellow solid,7aS)-2-Fluorotetrahydro-1H-pyrincycline-7a(5H)-yl)methoxy)quinazoline (145 mg, 75%) and the next reaction was carried out without further purification. LC/MS(ESI): m/z=648.3 [M+H]+.

### Step 6: 7-(8-fluoronaphthyl)-8-fluoro-4-(((R)-2-methylpiperazine)-1-yl))-2-(((2R,7aS)-2-Fluorotetrahydro-1H-pyrinyl-7a(5H)-yl)methoxy)quinazoline

7-(8-Fluoronaphthyl)-8-fluoro-4-(((R)-4-boc-2-methylpiperazin)-1-yl))-2-(((2R,7aS)-2-Fluorotetrahydro-1H-pyrincyclo-7a(5H)-yl)methoxy)quinazoline (129 mg, 0.2 mmol) in 1 ml ethyl acetate and 1N HCl in 1,4-dioxane Ring solution 2 ml. After stirring at room temperature for 2 hours, the reaction solution was neutralized with 1N sodium hydroxide solution and extracted with ethyl acetate. The obtained organic phase was washed with saturated sodium bicarbonate and saturated brine, dried over anhydrous sodium sulfate, and the organic phase was evaporated to dryness under reduced pressure. The compound 7-(8-fluoronaphthyl)-8-fluoro-4-(((R)-2-methylpiperazine)-1-yl))-2-(((2R,7aS)-2-Fluorotetrahydro-1H-pyrincycl-7a(5H)-yl)methoxy)quinazoline (94 mg, 86% yield) was used directly in the next step. LC/MS (ESI): m/z=548.3 [M+H]+.

### Step 7: 7-(8-fluoronaphthyl)-4-(((R)-4-acryloyl-2-methylpiperazine)-1-yl))-8-fluoro-2-((((2R,7aS)-2-fluorotetrahydro-1H-pyrinyl-7a(5H)-yl)methoxy)quinazoline

Add 7-(8-fluoronaphthyl)-8-fluoro-4-(((R)-2-methylpiperazine)-1-yl))-2-(((2R,7aS) to the reaction flask -2-Fluorotetrahydro-1H-pyrincyclo-7a(5H)-yl)methoxy)quinazoline (82 mg, 0.15 mmol), triethylamine (20.4 mg, 0.2 mmol), 4 ml tetrahydrofuran, ice water bath After cooling, a solution of 2-acryloyl chloride (18 mg, 0.2 mmol) in 0.5 ml of tetrahydrofuran was slowly added dropwise. After the addition was complete, stirring was continued for 4 hours. The reaction solution was quenched with methanol and evaporated to dryness under reduced pressure. The residue was purified by column chromatography to give compound 47 (43 mg, 48% yield) as a yellow solid. LC/MS(ESI): m/z=602.3 [M+H]+.

### EXAMPLE 48

7-(3-Hydroxy-8-fluoronaphthyl)-8-fluoro-4-(((R)-4-acryloyl-2-methylpiperazine)-1-yl)-2-(((2R, 7aS)-2-fluorotetrahydro-1H-pyrincycline-7a(5H)-yl)methoxy)quinazoline (compound 48)

Compound 48 was obtained in a similar manner to Example 47 (55 mg, 59% yield). LC/MS (ESI): m/z=618.3 [M+H]+.

### EXAMPLE 49

7-(3-Hydroxy-8-fluoronaphthyl)-8-fluoro-4-(((R)-4-acryloyl-2-methylpiperazine)-1-yl)-2-((tetrahydro-1H-pyrinyl-7a(5H)-yl)methoxy)quinazoline (compound 49)

Compound 49 was obtained in a similar manner to Example 47 (59 mg, 66% yield). LC/MS (ESI): m/z=600.3 [M+H]+.

### EXAMPLE 50

7-(8-fluoronaphthyl)-8-fluoro-4-(((R)-4-acryloyl-2-methylpiperazine)-1-yl)-2-((tetrahydro-1H-pyrinecyclo-7a(5H)-yl)methoxy)quinazoline (Compound 50)

Compound 50 was obtained in a similar manner to Example 47 (56 mg, 64% yield). LC/MS (ESI): m/z=584.3 [M+H]+.

### EXAMPLE 51

7-(8-fluoronaphthyl)-8-fluoro-4-(((S)-4-(2-fluoroacryloyl)-3-carbonitrileethylpiperazine)-1-yl)-2-(((2R,7aS)-2-fluorotetrahydro-1H-pyrinyl-7a(5H)-yl)methoxy)quinazoline (Compound 51)

Compound 51 was obtained in a similar manner to Example 47 (51 mg, 54% yield). LC/MS (ESI): m/z=627.3 [M+H]+.

### EXAMPLE 52

7-(3-Hydroxy-8-fluoronaphthyl)-8-fluoro-4-(((S)-4-(2-fluoroacryloyl)-3-carbonitrileethylpiperazine)-2-(((2R,7aS)-2-fluorotetrahydro-1H-pyrinyl-7a(5H)-yl)methoxy)quinazoline (Compound 52)

Compound 52 was obtained in a similar manner to Example 47 (57 mg, 59% yield). LC/MS (ESI): m/z=643.3 [M+H]+.

### EXAMPLE 53

7-(3-hydroxy-8-fluoronaphthyl)-8-fluoro-4-(((S)-4-(2-fluoroacryloyl)-3-carbonitrileethylpiperazine)-2-(((2R,7aS)-2-fluorotetrahydro-1H-pyrinyl-7a(5H)-yl)methoxy)quinazoline (Compound 53)

Compound 53 was obtained in a similar manner to Example 47 (33 mg, 33% yield). LC/MS (ESI): m/z=661.3 [M+H]+.

### EXAMPLE 54

7-(8-fluoronaphthyl)-8-fluoro-4-(((S)-4-(acryloyl)-3-carbonitrileethylpiperazine)-1-yl)-2-((tetrahydro-1H-pyrin-7a(5H)-yl)methoxy)quinazoline (compound 54)

Compound 54 was obtained in a similar manner to Example 47 (40 mg, 41% yield). 1H NMR (400 MHz, CDCl3)δ: 7.98(td,1H), 7.91(d,1H) 7.81-7.70(m,2H), 7.64-7.40(m,3H), 7.11(m,1H), 5.57-5.23(m,3H),4.91-4.84(m,1H),4.58-4.42(m,3H),4.29(d,1H),4.19-3.77(m,3H),3.52(br,1H),3.20-2.75 (m, 4H), 2.68-2.54 (m, 2H), 2.22-2.12 (m, 1H), 2.01-1.69 (m, 5H); LC/MS (ESI): m/z=645.3 [M+H]+.

### EXAMPLE 55

7-(3-hydroxy-8-fluoronaphthyl)-8-fluoro-4-(((S)-4-(acryloyl)-3-carbonitrileethylpiperazine)-2-((tetrahydro-1H- pyrincyclo-7a(5H)-yl)methoxy)quinazoline (compound 55)

Compound 55 was obtained in a similar manner to Example 47 (52 mg, 56% yield). LC/MS (ESI): m/z=625.3 [M+H]+.

### EXAMPLE 56

7-(8-fluoronaphthyl)-8-fluoro-4-(((S)-4-(acryloyl)-3-carbonitrileethylpiperazine)-1-yl)-2-((tetrahydro-1H pyrincyclo-7a(5H)-yl)methoxy)quinazoline (compound 56)

Compound 56 was obtained in a similar manner to example 47 (56 mg, 61% yield). LC/MS (ESI): m/z=609.3 [M+H]+.

### EXAMPLE 57

7-(3-Hydroxy-8-fluoronaphthyl)-8-fluoro-4-(((S)-4-(2-fluoroacryloyl)-3-carbonitrileethylpiperazine)-2-((tetrahydro-1H-pyrinyl-7a(5H)-yl)methoxy)quinazoline (compound 57)

Compound 57 was obtained in a similar manner to Example 47 (37 mg, 38% yield). LC/MS (ESI): m/z=643.3 [M+H]+.

### EXAMPLE 58

7-(8-fluoronaphthyl)-8-fluoro-4-(((S)-4-(2-fluoroacryloyl)-3-carbonitrileethylpiperazine)-1-yl)-2-((tetrakis Preparation of Hydro-1H-Pyrin-7a(5H)-yl)methoxy)quinazoline (Compound 58)

Compound 58 was obtained in a similar manner to Example 47 (34 mg, 36% yield). LC/MS (ESI): m/z=627.3 [M+H]+.

### EXAMPLE 59

7-(8-fluoronaphthyl)-8-fluoro-4-(((R)-4-acryloyl-2-methylpiperazine)-1-yl)-2-(((S)-2,2-difluorotetrahydro-1H-pyrincyclo-7a(5H)-yl)methoxy)quinazoline (59)

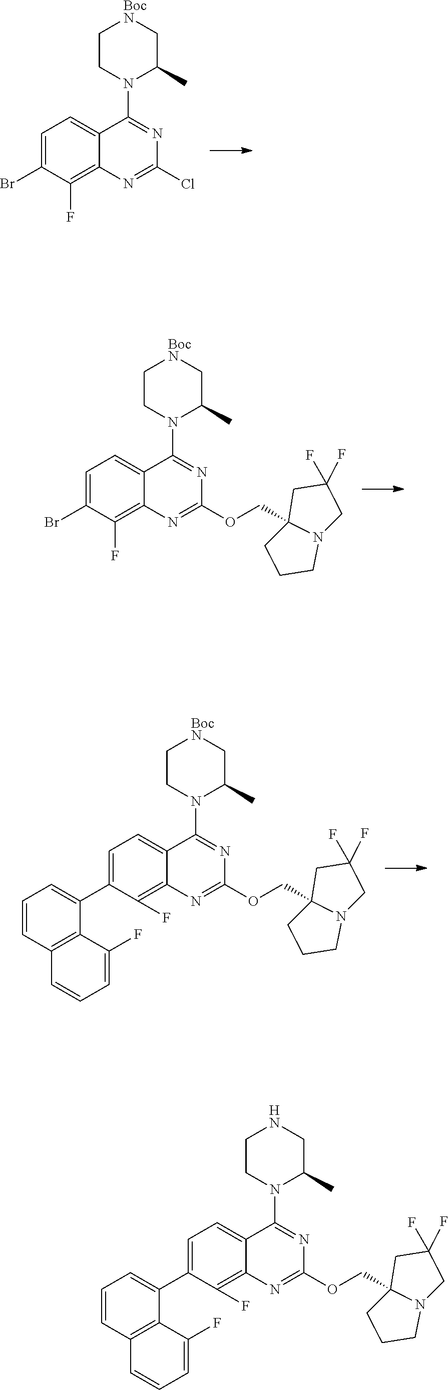 [SMILES: C[C@@H]1CN(C(=O)OC(C)(C)C)CCN1c1nc(Cl)nc2c(F)c(Br)ccc12 ; C[C@@H]1CN(C(=O)OC(C)(C)C)CCN1c1nc(OC[C@@]23CCCN2CC(F)(F)C3)nc2c(F)c(Br)ccc12 ; C[C@@H]1CN(C(=O)OC(C)(C)C)CCN1c1nc(OC[C@@]23CCCN2CC(F)(F)C3)nc2c(F)c(-c3cccc4cccc(F)c34)ccc12 ; C[C@@H]1CNCCN1c1nc(OC[C@@]23CCCN2CC(F)(F)C3)nc2c(F)c(-c3cccc4cccc(F)c34)ccc12]

### Step 1: 7-bromo-8-fluoro-4-(((R)-4-boc-2-methylpiperazine)-1-yl))-2-(((S)-2,2-Difluorotetrahydro-1H-pyrinyl-7a(5H)-yl)methoxy)quinazoline

7-Bromo-8-fluoro-4-((((R)-4-boc-2-methylpiperazine)-1-yl))quinazoline (276 mg, 0.6 mmol), (8S)-2, 2-Difluorotetrahydro-1H-pyrinyl-7a(5H)-yl)methanol (117 mg, 0.66 mmol), potassium carbonate (124 mg, 0.90 mmol) catalytic potassium iodide and DMF (20 mL) were mixed and heated to 120° C., and the reaction was stirred for 4 hours. Cooled to room temperature, evaporated under reduced pressure, and obtained by column chromatography as a yellow solid 7-bromo-8-fluoro-4-(((R)-4-boc-2-methylpiperazine)-1-yl))-2-(((S)-2,2-Difluorotetrahydro-1H-pyrinyl-7a(5H)-yl)methoxy)quinazoline (327 mg, 91%). LC/MS (ESI): m/z=601.2 [M-FH]+.

### Step 2: 7-(8-fluoronaphthyl)-8-fluoro-4-(((R)-4-boc-2-methylpiperazine)-1-yl))-2-(((S)-2,2-difluorotetrahydro-1H-pyrinyl-7a(5H)-yl)methoxy)quinazoline

7-Bromo-8-fluoro-4-(((R)-4-boc-2-methylpiperazine)-1-yl))-2-(((S)-2,2-difluorotetra Hydro-1H-pyrin-7a(5H)-yl)methoxy)quinazoline (271 mg, 0.45 mmol), 8-fluoronaphthalene-1-boronic acid (86 mg, 0.45 mmol), tris(dibenzylidene) Acetone)dipalladium (0.04 g, 0.04 mmol), cesium carbonate, 1,4-dioxane (6 mL) and water (1.5 mL) were mixed, then heated to 120° C. under reflux, and the reaction was stirred for 16 hours. The reaction was cooled to room temperature and stirred overnight to give a pale yellow precipitate. The reaction mixture was diluted with water (2 mL) and the solid was collected by filtration. Dry to give a yellow solid 7-(8-fluoronaphthyl)-8-fluoro-4-(((R)-4-boc-2-methylpiperazin)-1-yl))-2-WS)-2,2-difluorotetrahydro-1H-pyrincycline-7a(5H)-yl)methoxy)quinazoline (234 mg, 78%) and the next reaction was carried out without further purification. LC/MS(ESI): m/z=666.2 [M+H]+.

### Step 3: 7-(8-fluoronaphthyl)-8-fluoro-4-(((R)-2-methylpiperazine)-1-yl))-2-(((S)-2, 2-difluorotetrahydro-1H-pyrinyl-7a(5H)-yl)methoxy)quinazoline

7-(8-Fluoronaphthyl)-8-fluoro-4-(((R)-4-boc-2-methylpiperazine)-1-yl))-2-(((S)-2, 2-difluorotetrahydro-1H-pyrinyl-7a(5H)-yl)methoxy)quinazoline (199 mg, 0.3 mmol) was dissolved in 1 ml of ethyl acetate and 1N HCl in 1,4-dioxane Six ring solution 2 ml. After stirring at room temperature for 2 hours, the reaction solution was neutralized with IN sodium hydroxide solution and extracted with ethyl acetate. The obtained organic phase was washed with saturated sodium bicarbonate and saturated brine, dried over anhydrous sodium sulfate, and the organic phase was evaporated to dryness under reduced pressure. The compound 7-(8-fluoronaphthyl)-8-fluoro-4-(((R)-2-methylpiperazine)-1-yl))-2-(((S)-2,2-difluorotetrahydro-1H-pyrincycl-7a(5H)-yl)methoxy)quinazoline (144 mg, 85% yield) was used directly in the next step. LC/MS (ESI): m/z=566.2 [M+H]+.

### Step 4: 7-(8-fluoronaphthyl)-4-(((R)-4-acryloyl-2-methylpiperazine)-1-yl))-8-fluoro-2-((((S)-2,2-difluorotetrahydro-1H-pyrincycline-7a(5H)-yl)methoxy)quinazoline

Add 7-(8-fluoronaphthyl)-8-fluoro-4-(((R)-2-methylpiperazine)-1-yl))-2-(((S)-2 to the reaction flask, 2-difluorotetrahydro-1H-pyrinyl-7a(5H)-yl)methoxy)quinazoline (113 mg, 0.2 mmol), triethylamine (30 mg, 0.3 mmol), 5 ml tetrahydrofuran, ice water bath After cooling, a solution of 2-acryloyl chloride (27 mg, 0.3 mmol) in 0.5 ml of tetrahydrofuran was slowly added dropwise. After the addition was complete, stirring was continued for 4 hours. The reaction solution was quenched with methanol and evaporated to dryness under reduced pressure. The residue was purified by column chromatography to give compound 59 (71 mg, yield 57%) as a yellow solid. LC/MS(ESI): m/z=621.2 [M+H]+.

### EXAMPLE 60

7-(3-Hydroxy-8-fluoronaphthyl)-8-fluoro-4-(((R)-4-acryloyl-2-methylpiperazine)-1-yl)-2-(((S)-2,2-difluorotetrahydro-1H-pyrinyl-7a(5H)-yl)methoxy)quinazoline (compound 60)

Compound 60 was obtained in a similar manner to Example 59 (61 mg, 48% yield). LC/MS (ESI): m/z=637.2 [M+H]+.

### EXAMPLE 61

7-(8-Methylnaphthyl)-8-fluoro-4-(((R)-4-acryloyl-2-methylpiperazine)-1-yl)-2-(((2R,7aS)tetrakishydrogen-1H-pyrinyl-7a(5H)-yl)methoxy)quinazoline (Compound 61)

Compound 61 was obtained in a similar manner to Example 59 (65 mg, 54% yield). LC/MS (ESI): m/z=599.2 [M+H]+.

### EXAMPLE 62

7-(8-Methylnaphthyl)-8-fluoro-4-(((R)-4-acryloyl-2-methylpiperazine)-1-yl)-2-(((S)-2,2-difluorotetrahydro-1H-pyrinyl-7a(5H)-yl)methoxy)quinazoline (compound 62)

Compound 62 was obtained in a similar manner to Example 59 (77 mg, 63% yield). LC/MS (ESI): m/z=617.2 [M+H]+.

### EXAMPLE 63

7-(8-Fluoronaphthyl)-8-fluoro-4-(((S)-4-acryloyl-3-carbonitrileethylpiperazine)-1-yl)-2-(((S)-2,2-Difluorotetrahydro-1H-pyrinyl-7a(5H)-yl)methoxy)quinazoline (Compound 63)

Compound 63 was obtained in a similar manner to Example 59 (76 mg, 59% yield). LC/MS (ESI): m/z=646.2 [M+H]+.

### EXAMPLE 64

7-(3-Hydroxy-8-fluoronaphthyl)-8-fluoro-4-(((S)-4-acryloyl-3-carbonitrileethylpiperazine)-2-(((S)-2,2-difluorotetrahydro-1H-pyrincyclo-7a(5H)-yl)methoxy)quinazoline (compound 64)

Compound 64 (70 mg, 53% yield) was obtained in a similar manner to Example 59. LC/MS (ESI): m/z=662.2 [M+H]+.

### EXAMPLE 65

7-(3-Hydroxy-8-fluoronaphthyl)-8-fluoro-4-(((S)-4-(2-fluoroacryloyl)-3-carbonitrileethylpiperazine)-2-((((S)-2,2-Difluorotetrahydro-1H-pyrincycline-7a(5H)-yl)methoxy)quinazoline (Compound 65)

Compound 65 (77 mg, 57% yield) was obtained in a similar manner to Example 59. LC/MS (ESI): m/z=680.2 [M+H]+.

### EXAMPLE 66

7-(8-Fluoronaphthyl)-8-fluoro-(((S)-4-(2-fluoroacryloyl)-3-carbonitrileethylpiperazine)-1-yl)-2-((((S)-2,2-difluorotetrahydro-1H-pyrincycline-7a(5H)-yl)methoxy)quinazoline (compound 66)

Compound 66 (74 mg, 56% yield) was obtained in a similar manner to Example 59. LC/MS (ESI): m/z=664.2 [M+H]+.

### EXAMPLE 67

7-(8-Methylnaphthyl)-8-fluoro-4-(((S)-4-acryloyl-3-carbonitrileethylpiperazine)-2-(((S)-2,2-difluoro Preparation of Tetrahydro-1H-Pyrin-7a(5H)-yl)methoxy)quinazoline (Compound 67)

Compound 67 (67 mg, 52% yield) was obtained in a similar manner to Example 59. LC/MS (ESI): m/z=641.3 [M-FH]+.

### EXAMPLE 68

7-(8-Methylnaphthyl)-8-fluoro-4-(((S)-4-(2-fluoroacryloyl)-3-carbonitrileethylpiperazine)-1-yl)-2-(((S)-2,2-difluorotetrahydro-1H-pyrinyl-7a(5H)-yl)methoxy)quinazoline (Compound 68)

Compound 68 (82 mg, 62% yield) was obtained in a similar manner to Example 59. LC/MS (ESI): m/z=659.2 [M-FH]+.

### EXAMPLE 69

7-(8-Fluoronaphthyl)-8-fluoro-4-(((R)-4-acryloyl-2-methylpiperazine)-1-yl)-2-(((2R,7aS)-2-fluorotetrahydro-1H-pyrincyclo-7a(5H)-yl)methoxy)quinazoline (69)

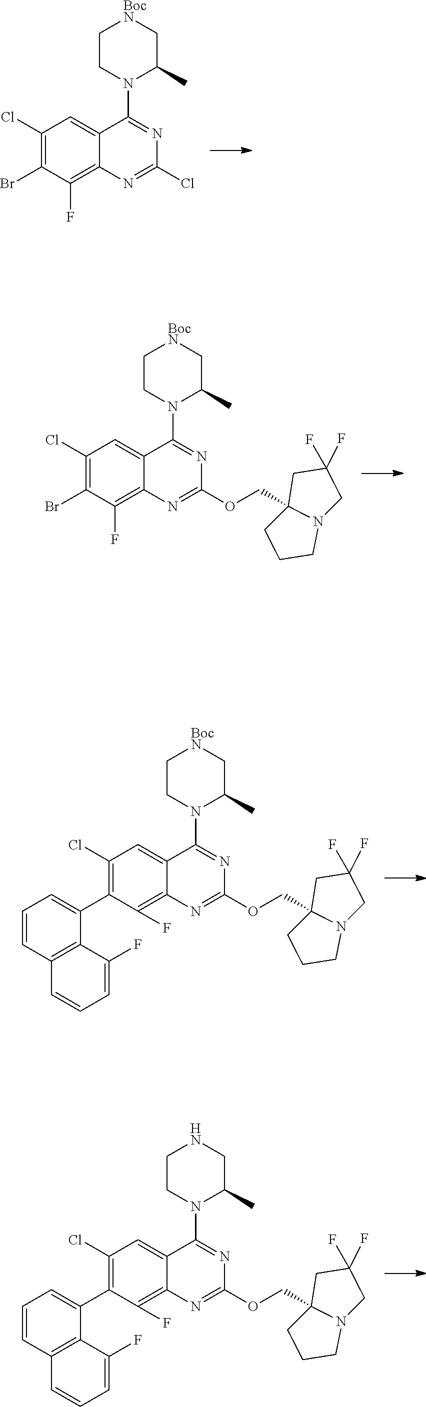 [SMILES: C[C@@H]1CN(C(=O)OC(C)(C)C)CCN1c1nc(Cl)nc2c(F)c(Br)c(Cl)cc12 ; C[C@@H]1CN(C(=O)OC(C)(C)C)CCN1c1nc(OC[C@@]23CCCN2CC(F)(F)C3)nc2c(F)c(Br)c(Cl)cc12 ; C[C@@H]1CN(C(=O)OC(C)(C)C)CCN1c1nc(OC[C@@]23CCCN2CC(F)(F)C3)nc2c(F)c(-c3cccc4cccc(F)c34)c(Cl)cc12 ; C[C@@H]1CNCCN1c1nc(OC[C@@]23CCCN2CC(F)(F)C3)nc2c(F)c(-c3cccc4cccc(F)c34)c(Cl)cc12]

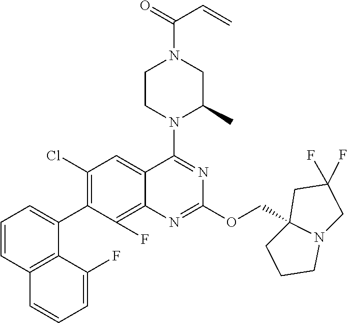 [SMILES: C=CC(=O)N1CCN(c2nc(OC[C@@]34CCCN3CC(F)(F)C4)nc3c(F)c(-c4cccc5cccc(F)c45)c(Cl)cc23)[C@H](C)C1]

### Step 1: 6-chloro-7-bromo-8-fluoro-4-(((R)-4-boc-2-methylpiperazine)-1-yl))-2-(((S)-2,2-difluorotetrahydro-1H-pyrinyl-7a(5H)-yl)methoxy)quinazoline

6-Chloro-7-bromo-8-fluoro-4-(((R)-4-boc-2-methylpiperazine)-1-yl))quinazoline (498 mg, 1 mmol), (8S)-2,2-Difluorotetrahydro-1H-pyrinyl-7a(5H)-yl)methanol (195 mg, 1.1 mmol), potassium carbonate (207 mg, 1.5 mmol) catalytic potassium iodide and DMF (20 mL) were mixed and heated To 120° C., the reaction was stirred for 4 hours. Cooled to room temperature, evaporated under reduced pressure, and column chromatography gave yellow solid 6-chloro-7-bromo-8-fluoro-4-(((R)-4-boc-2-methylpiperazine)-1-yl))-2-(((S)-2,2-difluorotetrahydro-1H-pyrincycl-7a(5H)-yl)methoxy)quinazoline (590 mg, 93%). LC/MS (ESI): m/z=636.1 [M+H]+.

### Step 2: 6-chloro-7-(8-fluoronaphthyl)-8-fluoro-4-(((R)-4-boc-2-methylpiperazine)-1-yl))-2- WS)-2,2-difluorotetrahydro-1H-pyrincycline-7a(5H)-yl)methoxy)quinazoline

6-Chloro-7-bromo -8-fluoro-4-(((R)-4-boc -2-methylpiperazine)-1-yl))-2-(((S)-2,2-Difluorotetrahydro-1H-pyrinyl-7a(5H)-yl)methoxy)quinazoline (381 mg, 0.6 mmol), 8-fluoronaphthalene-1-boronic acid (114 mg, 0.6 mmol), tris(Dibenzylideneacetone) dipalladium (0.052 g, 0.054 mmol), cesium carbonate, 1,4-dioxane (12 mL) and water (3 mL) were mixed, then heated to 120° C. under reflux, and the reaction was stirred for 16 hours. The reaction was cooled to room temperature and stirred overnight to give a pale yellow precipitate. The reaction mixture was diluted with water (4 mL) and the solid was collected by filtration. Dry to give a yellow solid 7-(8-fluoronaphthyl)-8-fluoro-4-(((R)-4-boc-2-methylpiperazin)-1-yl))-2-WS)-2,2-diflu orotetrahydro-1H-pyrincyclin-7a(5H)-yl)methoxy)quinazoline (298 mg, 71%) and the next reaction was carried out without further purification. LC/MS (ESI): m/z=701.2 [M+H]+.

### Step 3: 6-chloro-7-(8-fluoronaphthyl)-8-fluoro-4-(((R)-2-methylpiperazine)-1-yl))-2-(((S)-2,2-difluorotetrahydro-1H-pyrinyl-7a(5H)-yl)methoxy)quinazoline

6-Chloro-7-(8-fluoronaphthyl)-8-fluoro-4-(((R)-4-boc-2-methylpiperazine)-1-yl))-2-((((S)-2,2-difluorotetrahydro-1H-pyrincycline-7a(5H)-yl)methoxy)quinazoline (280 mg, 0.4 mmol) was dissolved in 2 ml of ethyl acetate and 1 N HCl in 1, 4-Dioxane solution 4 ml. After stirring at room temperature for 2 hours, the reaction solution was neutralized with 1N sodium hydroxide solution and extracted with ethyl acetate. The obtained organic phase was washed with saturated sodium bicarbonate and saturated brine, dried over anhydrous sodium sulfate, and the organic phase was evaporated to dryness under reduced pressure. The compound 6-chloro-7-(8-fluoronaphthyl)-8-fluoro-4-(((R)-2-methylpiperazine)-1-yl))-2-(((S)-2,2-Difluorotetrahydro-1H-pyrincycl-7a(5H)-yl)methoxy)quinazoline (209 mg, 87% yield) was used directly in the next step. LC/MS (ESI): m/z=601.1 [M+H]+.

### Step 4: 6-chloro-7-(8-fluoronaphthyl)-4-(((R)-4-acryloyl-2-methylpiperazine)-1-yl))-8-fluoro-2-(((S)-2,2-difluorotetrahydro-1H-pyrinyl-7a(5H)-yl)methoxy)quinazoline

Add 6-chloro-7-(8-fluoronaphthyl)-8-fluoro-4-(((R)-2-methylpiperazine)-1-yl))-2-((((S)-2,2-Difluorotetrahydro-1H-pyrinyl-7a(5H)-yl)methoxy)quinazoline (180 mg, 0.3 mmol), triethylamine (40.8 mg, 0.4 mmol), 8 ml of tetrahydrofuran, cooled in an ice-water bath, and slowly added dropwise a solution of 2-acryloyl chloride (36 mg, 0.4 mmol) in 1 ml of tetrahydrofuran. After the addition was complete, stirring was continued for 4 hours. The reaction solution was quenched with methanol and evaporated to dryness under reduced pressure. The residue was purified by column chromatography to give compound 69 (104 mg, 53% yield) as a yellow solid. LC/MS(ESI): m/z=655.2 [M+H]+.

### EXAMPLE 70

7-(3-Hydroxy-8-fluoronaphthyl)-8-fluoro-4-(((R)-4-acryloyl-2-methylpiperazine)-1-yl)-2-(((S)-2,2-difluorotetrahydro-1H-pyrincycline-7a(5H)-yl)methoxy)quinazoline (compound 70)

Compound 70 was obtained in a similar manner to Example 69 (115 mg, 57% yield). LC/MS (ESI): m/z=671.2 [M+H]+.

### EXAMPLE 71

7-(8-Methylnaphthyl)-8-fluoro-4-(((S)-4-acryloyl-3-carbonitrileethylpiperazine)-2-(((S)-2,2-difluorotetrahydro-1H-pyrinyl-7a(5H)-yl)methoxy)quinazoline (Compound 71)

Compound 71 was obtained in a similar manner to Example 69 (95 mg, 47% yield). LC/MS (ESI): m/z=676.2 [M+H]+.

### EXAMPLE 72

7-(8-Methylnaphthyl)-8-fluoro-4-(((S)-4-(2-fluoroacryloyl)-3-carbonitrileethylpiperazine)-1-yl)-2-(((S)-2,2-difluorotetrahydro-1H-pyrincyclo-7a(5H)-yl)methoxy)quinazoline (Compound 72)

Compound 72 was obtained in a similar manner to Example 69 (87 mg, 42% yield). LC/MS (ESI): m/z=694.2 [M+H]+.

### EXAMPLE 73

6-Chloro-7-(8-methylnaphthyl)-8-fluoro-4-(((R)-4-acryloyl-2-methylpiperazine)-1-yl)-2-(((2R,7aS)-2-fluorotetrahydro-1H-pyrinyl-7a(5H)-yl)methoxy)quinazoline (compound 73)

Compound 73 was obtained in a similar manner to Example 69 (102 mg, 54% yield). LC/MS (ESI): m/z=633.3 [M+H]+.

### EXAMPLE 74

6-Chloro-7-(8-methylnaphthyl)-8-fluoro-4-(((R)-4-acryloyl-2-methylpiperazine)-1-yl)-2-((((S))-2,2-Difluorotetrahydro-1H-pyrincycline-7a(5H)-yl)methoxy)quinazoline (Compound 74)

Compound 74 was obtained in a similar manner to Example 69 (105 mg, 54% yield). LC/MS (ESI): m/z=651.2 [M+H]+.

### EXAMPLE 75

6-Chloro-7-(8-fluoronaphthyl)-8-fluoro-4-(((S)-4-acryloyl-3-carbonitrileethylpiperazine)-2-(((S)-2,2- difluorotetrahydro-1H-pyrincyclo-7a(5H)-yl)methoxy)quinazoline (compound 75)

Compound 75 (116 mg, 57% yield) was obtained in a similar manner to Example 69. LC/MS (ESI): m/z=680.2 [M+H]+.

### EXAMPLE 76

6-Chloro-7-(3-hydroxy-8-fluoronaphthyl)-8-fluoro-4-(((S)-4-acryloyl-3-carbonitrileethylpiperazine)-1-yl)-2-(((S)-2,2-difluorotetrahydro-1H-pyrinyl-7a(5H)-yl)methoxy)quinazoline (Compound 76)

Compound 76 (131 mg, 63% yield) was obtained in a similar manner to Example 69. LC/MS (ESI): m/z=696.2 [M+H]+.

### EXAMPLE 77

6-Chloro-7-(3-hydroxy-8-fluoronaphthyl)-8-fluoro-4-(((S)-4-(2-fluoroacryloyl)-3-carbonitrileethylpiperazine)-1-yl)-2-(((S)-2,2-difluorotetrahydro-1H-pyrincycline-7a(5H)-yl)methoxy)quinazoline (Compound 75)

Compound 77 was obtained in a similar manner to Example 69 (88 mg, 41% yield). LC/MS (ESI): m/z=714.2 [M+H]+.

### EXAMPLE 78

6-Chloro-7-(8-fluoronaphthyl)-8-fluoro-4-(((S)-4-(2-fluoroacryloyl)-3-carbonitrileethylpiperazine)-1-yl)-2-(((S)-2,2-difluorotetrahydro-1H-pyrincycline-7a(5H)-yl)methoxy)quinazoline (Compound 78)

Compound 78 was obtained in a similar manner to Example 69 (81 mg, 39% yield). LC/MS (ESI): m/z=698.2 [M+H]+.

### EXAMPLE 79

7-(8-Fluoronaphthyl)-6-fluoro-4-(((R)-4-acryloyl-2-methylpiperazine)-1-yl)-2-(((S)-2,2-difluorotetrahydro-1H-pyrincycl-7a(5H)-yl)methoxy)pyridine[2,3-d]lopyrimidine (79)

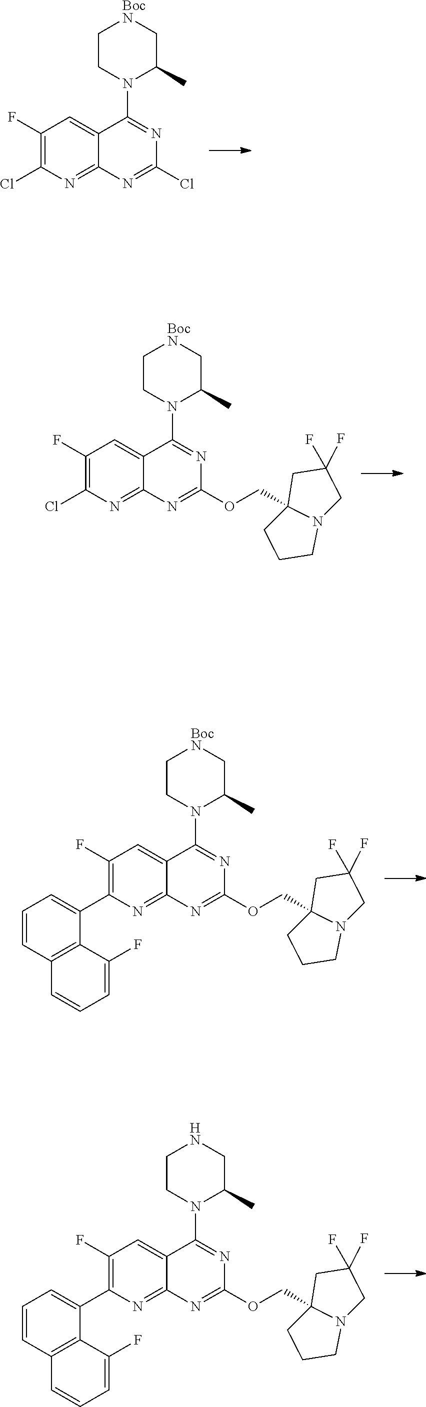 [SMILES: C[C@@H]1CN(C(=O)OC(C)(C)C)CCN1c1nc(Cl)nc2nc(Cl)c(F)cc12 ; C[C@@H]1CN(C(=O)OC(C)(C)C)CCN1c1nc(OC[C@@]23CCCN2CC(F)(F)C3)nc2nc(Cl)c(F)cc12 ; C[C@@H]1CN(C(=O)OC(C)(C)C)CCN1c1nc(OC[C@@]23CCCN2CC(F)(F)C3)nc2nc(-c3cccc4cccc(F)c34)c(F)cc12 ; C[C@@H]1CNCCN1c1nc(OC[C@@]23CCCN2CC(F)(F)C3)nc2nc(-c3cccc4cccc(F)c34)c(F)cc12]

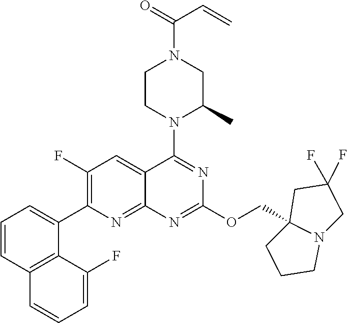 [SMILES: C=CC(=O)N1CCN(c2nc(OC[C@@]34CCCN3CC(F)(F)C4)nc3nc(-c4cccc5cccc(F)c45)c(F)cc23)[C@H](C)C1]

### Step 1: 7-chloro-6-fluoro-4-(((R)-4-boc-2-methylpiperazine)-1-yl))-2-(((S)-2,2-Difluorotetrahydro-1H-pyrincycl-7a(5H)-yl)methoxy)pyridine[2,3-d]lopyrimidine

2,7-Dichloro-6-fluoro-4-(((R)-4-boc-2-methylpiperazin)-1-yl))pyridine[2,3-d]lopyrimidine (416 mg, 1 mmol), (8S)-2,2-difluorotetrahydro-1H-pyrinyl-7a(5H)-yl)methanol (195 mg, 1.1 mmol), potassium carbonate (207 mg, 1.5 mmol) catalytic potassium iodide and DMF (20 mL) was mixed, heated to 120° C., and the reaction was stirred for 4 hours. Cool to room temperature, evaporate under reduced pressure, and obtain yellow solid 6-fluoro-7-(8-fluoronaphthyl)-4-(((R)-4-boc-2-methylpiperazine)-1 by column chromatography-yl))-2-(((S)-2,2-difluorotetrahydro-1H-pyrincycl-7a(5H)-yl)methoxy)pyridine[2,3-d]lopyrimidine (485 mg, 87%). LC/MS (ESI): m/z=558.2 [M+H]+.

### Step 2: 6-fluoro-7-(8-fluoronaphthyl)-4-(((R)-4-boc-2-methylpiperazine)-1-yl))-2-(((S)-2,2-difluorotetrahydro-1H-pyrinc ycline-7a(5H)-yl)methoxy)pyridine[2,3-d]lopyrimidine

7-Chloro-6-fluoro-4-(((R)-4-boc -2-methylpiperazine)-1-yl))-2-(((S)-2,2-difluorotetra Hydro-1H-pyrinyl-7a(5H)-yl)methoxy)pyridine[2,3-d]lopyrimidine 21b (334 mg, 0.6 mmol), 8-fluoronaphthalene-1-boronic acid (114 mg, 0.6 mmol)), tris(dibenzylideneacetone)dipalladium (0.054 g, 0.054 mmol), cesium carbonate, 1,4-dioxane (12 mL) and water (3 mL) were mixed, then heated to 120° C. under reflux, stirred The reaction was carried out for 16 hours. The reaction was cooled to room temperature and stirred overnight to give a pale yellow precipitate. The reaction mixture was diluted with water (4 mL) and the solid was collected by filtration. Dry to give a yellow solid 6-fluoro-7-(8-fluoronaphthyl)-4-(((R)-2-methylpiperazin)-1-yl))-2-(((S)-2, 2-Difluorotetrahydro-1H-pyrincycl-7a(5H)-yl)methoxy)pyridine[2,3-d]lopyrimidine (308 mg, 77%) was carried to the next reaction without further purification. LC/MS(ESI): m/z=667.3 [M+H]+.

### Step 3: 6-fluoro-7-(8-fluoronaphthyl)-4-(((R)-2-methylpiperazine)-1-yl))-2-(((S)-2, 2-Difluorotetrahydro-1H-pyrinyl-7a(5H)-yl)methoxy)pyridine[2,3-d]lopyrimidine

6-Fluoro-7-(8-fluoronaphthyl)-4-(((R)-2-methylpiperazine)-1-yl))-2-(((S)-2,2-di Fluorotetrahydro-1H-pyrincycl-7a(5H)-yl)methoxy)pyridine[2,3-d]lopyrimidine (267 mg, 0.4 mmol) was dissolved in 2 ml of ethyl acetate and 1N HCl in 1,4-Dioxane solution 4 ml. After stirring at room temperature for 2 hours, the reaction solution was neutralized with 1N sodium hydroxide solution and extracted with ethyl acetate. The obtained organic phase was washed with saturated sodium bicarbonate and saturated brine, dried over anhydrous sodium sulfate, and the organic phase was evaporated to dryness under reduced pressure. The compound 6-fluoro-7-(8-fluoronaphthyl)-4-(((R)-2-methylpiperazin)-1-yl))-2-(((S)-2,2-difluorotetrahydro-1H-pyrincycl-7a(5H)-yl)methoxy)pyridine[2,3-d]lopyrimidine (190 mg, 84% yield) was used directly in the next step. LC/MS (ESI): m/z=567.3 [M+H]+.

### Step 4: 6-fluoro-7-(8-fluoronaphthyl)-4-(((R)-4-acryloyl-2-methylpiperazine)-1-yl))-8-fluoro-2-(((S)-2,2-difluorotetrahydro-1H-pyrinyl-7a(5H)-yl)methoxy)pyridine[2,3-d]lopyrimidine

Add 6-fluoro-7-(8-fluoronaphthyl)-4-(((R)-2-methylpiperazine)-1-yl))-2-(((S)-2 to the reaction flask, 2-difluorotetrahydro-1H-pyrinyl-7a(5H)-yl)methoxy)pyridine[2,3-d]lopyrimidine (170 mg, 0.3 mmol), triethylamine (40.8 mg, 0.4 mmol), 8 ml of tetrahydrofuran, after cooling in an ice-water bath, a solution of 2-acryloyl chloride (36 mg, 0.4 mmol) in 1 ml of tetrahydrofuran was slowly added dropwise. After the addition was complete, stirring was continued for 4 hours. The reaction solution was quenched with methanol and evaporated to dryness under reduced pressure. The residue was purified by column chromatography to give compound 79 (99 mg, yield 53%) as a yellow solid. LC/MS(ESI): m/z=622.3 [M+H]+.

### EXAMPLE 80

7-(8-Methylnaphthyl)-6-fluoro-4-(((R)-4-acryloyl-2-methylpiperazine)-1-yl)-1-y1)-2-(((S)-2,2-difluorotetrahydro-1H-pyrincyclo-7a(5H)-yl)methoxy)pyridine[2,3-d]lopyrimidine (Compound 80)

Compound 80 (107 mg, 58% yield) was obtained in a similar manner to Example 79. LC/MS (ESI): m/z=618.3 [M+H]+.

### EXAMPLE 81

7-(8-Methylnaphthyl)-6-fluoro-4-(((R)-4-acryloyl-2-methylpiperazine)-1-yl)-2-(((2R,7aS)-2-fluorotetrahydro-1H-pyrincyclo-7a(5H)-yl)methoxy)pyridine[2,3-d]lopyrimidine compound 81)

Compound 81 (113 mg, 63% yield) was obtained in a similar manner to Example 79. LC/MS (ESI): m/z=600.3 [M+H]+.

### EXAMPLE 82

7-(8-Methylnaphthyl)-6-fluoro-4-(((S)-4-(2-fluoroacryloyl)-3-carbonitrileethylpiperazine)-1-yl)-2-(((2R,7aS)-2-fluorotetrahydro-1H-pyrinyl-7a(5H)-yl)methoxy)pyridine[2,3-d]lopyrimidine (Compound 82)

Compound 82 (75 mg, 38% yield) was obtained in a similar manner to Example 79. LC/MS (ESI): m/z=661.3 [M+H]+.

### EXAMPLE 83

7-(8-Fluoronaphthyl)-6-fluoro-4-(((S)-4-(2-fluoroacryloyl)-3-carbonitrileethylpiperazine)-1-yl)-2-((((S)-2,2-difluorotetrahydro-1H-pyrincycl-7a(5H)-yl)methoxy)pyridine[2,3-d]lopyrimidine (Compound 83)

Compound 83 (86 mg, 43% yield) was obtained in a similar manner to Example 79. LC/MS (ESI): m/z=664.2 [M+H]+.

### EXAMPLE 84

7-(3-Hydroxy-8-fluoronaphthyl)-6-fluoro-4-(((S)-4-(2-fluoroacryloyl)-3-carbonitrileethylpiperazine)-1-yl)-2-(((S)-2,2-difluorotetrahydro-1H-pyrinyl-7a(5H)-yl)methoxy)pyridine[2,3-d]lopyrimidine (Compound 84)

Compound 84 (77 mg, 38% yield) was obtained in a similar manner to Example 79. LC/MS (ESI): m/z=680.2 [M+H]+.

### EXAMPLE 85

7-(8-Methylnaphthyl)-6-fluoro-4-(((S)-4-(2-fluoro acryloyl)-3-carbonitrileethylpiperazine)-1-yl)-2-(((S)-2,2-difluorotetrahydro-1H-pyrincycl-7a(5H)-yl)methoxy)pyridine[2,3-d]lopyrimidine (Compound 85)

Compound 85 (69 mg, 36% yield) was obtained in a similar manner to Example 79. LC/MS (ESI): m/z=642.3 [M+H]+.

### EXAMPLE 86

7-(8-Fluoronaphthyl)-4-(((R)-4-Acryloyl-2-methylpiperazine)-1-yl)-2-(((S)-2,2-difluorotetrakisHydro-1H-pyrincycl-7a(5H)-yl)methoxy)pyridine[2,3-d]lopyrimidine (86)

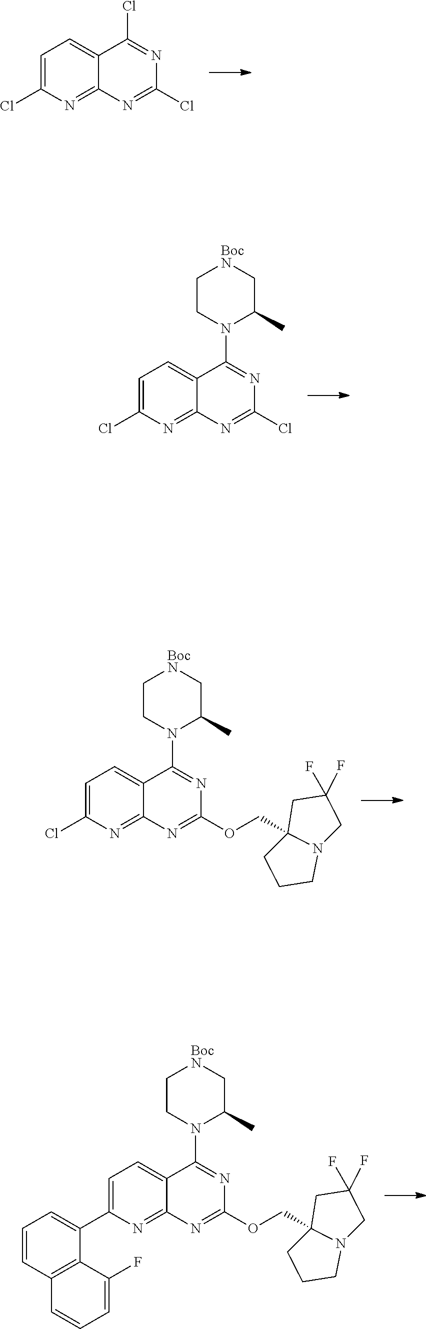 [SMILES: Clc1ccc2c(Cl)nc(Cl)nc2n1 ; C[C@@H]1CN(C(=O)OC(C)(C)C)CCN1c1nc(Cl)nc2nc(Cl)ccc12 ; C[C@@H]1CN(C(=O)OC(C)(C)C)CCN1c1nc(OC[C@@]23CCCN2CC(F)(F)C3)nc2nc(Cl)ccc12 ; C[C@@H]1CN(C(=O)OC(C)(C)C)CCN1c1nc(OC[C@@]23CCCN2CC(F)(F)C3)nc2nc(-c3cccc4cccc(F)c34)ccc12]

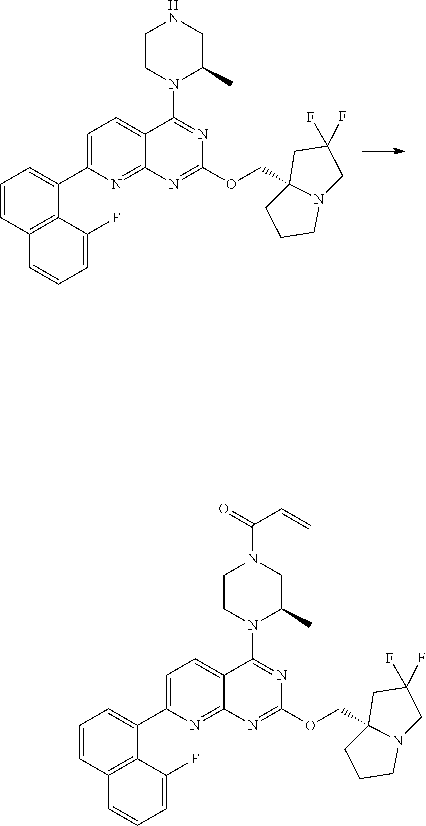 [SMILES: C[C@@H]1CNCCN1c1nc(OC[C@@]23CCCN2CC(F)(F)C3)nc2nc(-c3cccc4cccc(F)c34)ccc12 ; C=CC(=O)N1CCN(c2nc(OC[C@@]34CCCN3CC(F)(F)C4)nc3nc(-c4cccc5cccc(F)c45)ccc23)[C@H](C)C1]

### Step 1: 7-chloro-4-(((R)-4-boc-2-methylpiperazine)-1-yl))-2-(((S)-2,2-difluorotetrahydro Preparation of -1H-pyrinyl-7a(5H)-yl)methoxy)pyridine[2,3-d]lopyrimidine

2,7-Dichloro-4-(((R)-4-boc-2-methylpiperazin)-1-yl))pyridine[2,3-d]lopyrimidine (398 mg, 1 mmol), (8S)-2,2-difluorotetrahydro-1H-pyrinyl-7a(5H)-yl)methanol (195 mg, 1.1 mmol), potassium carbonate (207 mg, 1.5 mmol) catalytic potassium iodide and DMF (20 mL) were mixed, heated to 120° C., and stirred for 4 hours. Cooled to room temperature, evaporated under reduced pressure, and column chromatography gave a yellow solid 7-(8-fluoronaphthyl)-4-(((R)-4-boc-2-methylpiperazine)-1-yl))-2-(((S)-2,2-difluorotetrahydro-1H-pyrinyl-7a(5H)-yl)methoxy)pyridine[2,3-d]lopyrimidine (480 mg, 89%). LC/MS (ESI): m/z=540.2 [M+]+.

### Step 2: 7-(8-fluoronaphthyl)-4-(((R)-4-boc-2-methylpiperazine)-1-yl))-2-(((S)-2,2-Difluorotetrahydro-1H-pyrinyl-7a(5H)-yl)methoxy)pyridine[2,3-d]lopyrimidine

7-Chloro-4-(((R)-4-boc-2-methylpiperazine)-1-yl))-2-(((S)-2,2-difluorotetrahydro-1H-Pyrin-7a(5H)-yl)methoxy)pyridine[2,3-d]lopyrimidine (323 mg, 0.6 mmol), 8-fluoronaphthalene-1-boronic acid (114 mg, 0.6 mmol), tris(di) benzylideneacetone)dipalladium (0.054 g, 0.054 mmol), cesium carbonate, 1,4-dioxane (12 mL) and water (3 mL) were mixed, then heated to 120° C. under reflux, and the reaction was stirred for 16 hours. The reaction was cooled to room temperature and stirred overnight to give a pale yellow precipitate. The reaction mixture was diluted with water (4 mL) and the solid was collected by filtration. Drying gave 7-(8-fluoronaphthyl)-4-(((R)-2-methylpiperazin)-1-yl))-2-(((S)-2,2-difluoro) as a yellow solid Tetrahydro-1H-pyrincycl-7a(5H)-yl)methoxy)pyridine[2,3-d]lopyrimidine (307 mg, 79%) was carried to the next reaction without further purification. LC/MS (ESI): m/z=649.3 [M+H]+.

### Step 3: 7-(8-fluoronaphthyl)-4-(((R)-2-methylpiperazine)-1-yl))-2-WS)-2,2-difluorotetrahydro-1H-pyrinyl-7a(5H)-yl)methoxy)pyridine[2,3-d]lopyrimidine

7-(8-Fluoronaphthyl)-4-(((R)-2-methylpiperazine)-1-yl))-2-(((S)-2,2-difluorotetrahydro-1H-pyrincycl-7a(5H)-yl)methoxy)pyridine[2,3-d]lopyrimidine (259 mg, 0.4 mmol) was dissolved in 2 ml of ethyl acetate and 1N HCl in 1,4-dioxane Ring solution 4 ml. After stirring at room temperature for 2 hours, the reaction solution was neutralized with 1N sodium hydroxide solution and extracted with ethyl acetate. The obtained organic phase was washed with saturated sodium bicarbonate and saturated brine, dried over anhydrous sodium sulfate, and the organic phase was evaporated to dryness under reduced pressure. The compound 7-(8-fluoronaphthyl)-4-(((R)-2-methylpiperazine)-1-yl))-2-(((S)-2,2-difluorotetrahydro) was obtained -1H-Pyrincyclo-7a(5H)-yl)methoxy)pyridine[2,3-d]lopyrimidine (180 mg, 82% yield) was used directly in the next step. LC/MS (ESI): m/z=549.3 [M+H]+.

### Step 4: 7-(8-fluoronaphthyl)-4-(((R)-4-acryloyl-2-methylpiperazine)-1-yl))-2-(((S)-2,2-difluorotetrahydro-1H-pyrinyl-7a(5H)-yl)methoxy)pyridine[2,3-d]lopyrimidine

Add 7-(8-fluoronaphthyl)-4-(((R)-2-methylpiperazine)-1-yl))-2-(((S)-2,2-di into the reaction flask Fluorotetrahydro-1H-pyrincycl-7a(5H)-yl)methoxy)pyridine[2,3-d]lopyrimidine (165 mg, 0.3 mmol), triethylamine (40.8 mg, 0.4 mmol), 8 ml Tetrahydrofuran, after cooling in an ice-water bath, a solution of 2-acryloyl chloride (36 mg, 0.4 mmol) in 1 ml of tetrahydrofuran was slowly added dropwise. After the addition was complete, stirring was continued for 4 hours. The reaction solution was quenched with methanol and evaporated to dryness under reduced pressure. The residue was purified by column chromatography to give compound 86 (105 mg, 58% yield) as a yellow solid. LC/MS(ESI): m/z=603.3 [M+H]+.

### EXAMPLE 87

7-(8-Methylnaphthyl)-4-(((R)-4-acryloyl-2-methylpiperazine)-1-yl)-2-(((S)-2,2-difluoro tetrahydro-1H-pyrincycl-7a(5H)-yl)methoxy)pyridine[2,3-d]lopyrimidine (Compound 87)

Compound 87 was obtained in a similar manner to Example 86 (95 mg, 57% yield). LC/MS (ESI): m/z=559.3 [M+H]+.

### EXAMPLE 88

7-(8-Fluoronaphthyl)-8-fluoro-4-(((S)-4-(2-fluoro acryloyl)-3-c arbonitrileethylpiperazine)-1-yl)-2-((tetrakis Hydrogen-1H-pyrinyl-7a(5H)-yl)methoxy)quinazoline (Compound 88)

Compound 88 was obtained in a similar manner to Example 86 (98 mg, 56% yield). LC/MS (ESI): m/z=585.3 [M+H]+.

### EXAMPLE 89

7-(8-Methylnaphthyl)-4-(((R)-4-Acryloyl-2-methylpiperazine)-1-yl)-2-(((2R,7aS)-2-fluorotetrahydro-1H-pyrincycl-7a(5H)-yl)methoxy)pyridine[2,3-d]lopyrimidine (Compound 89)

Compound 89was obtained in a similar manner to Example 86 (106 mg, 61% yield). LC/MS (ESI): m/z=581.3 [M+H]+.

### EXAMPLE 90

7-(8-Fluoronaphthyl)-4-(((S)-4-(2-fluoroacryloyl)-3-carbonitrileethylpiperazine)-1-yl)-2-(((S)-2,2-difluorotetrahydro-1H-pyrincyclo-7a(5H)-yl)methoxy)pyridine[2,3-d]lopyrimidine (Compound 90)

Compound 90 was obtained in a similar manner to Example 86 (75 mg, 39% yield). LC/MS (ESI): m/z=646.2 [M+H]+.

### EXAMPLE 91

7-(3-Hydroxy-8-fluoronaphthyl)-4-(((S)-4-(2-fluoroacryloyl)-3-carbonitrileethylpiperazine)-1-yl)-2-(((S)-2,2-difluorotetrahydro-1H-pyrincycl-7a(5H)-yl)methoxy)pyridine[2,3-d]lopyrimidine (Compound 91)

Compound 91 was obtained in a similar manner to Example 86 (81 mg, 41% yield). LC/MS (ESI): m/z=662.2 [M+H]+.

### EXAMPLE 92

7-(8-Fluoronaphthyl)-4-(((S)-4-(2-fluoroacryloyl)-3-carbonitrileethylpiperazine)-1-yl)-2-(((2R,7aS)-2-fluorotetrahydro-1H-pyrincycl-7a(5H)-yl)methoxy)pyridine[2,3-d]lopyrimidine (Compound 92)

Compound 92 was obtained in a similar manner to Example 86 (81 mg, 43% yield). LC/MS (ESI): m/z=628.3 [M+H]+.

### EXAMPLE 93

7-(8-Methylnaphthyl)-4-(((S)-4-(2-fluoroacryloyl)-3-carbonitrileethylpiperazine)-1-yl)-2-((((2R,7aS)-2-fluorotetrahydro-1H-pyrinyl-7a(5H)-yl)methoxy)pyridine[2,3-d]lopyrimidine (Compound 93)

Compound 93 was obtained in a similar manner to Example 86 (69 mg, 37% yield). LC/MS (ESI): m/z=624.3 [M+H]+.

Similar to the synthetic route of Examples 1-93, the following compounds can be obtained:

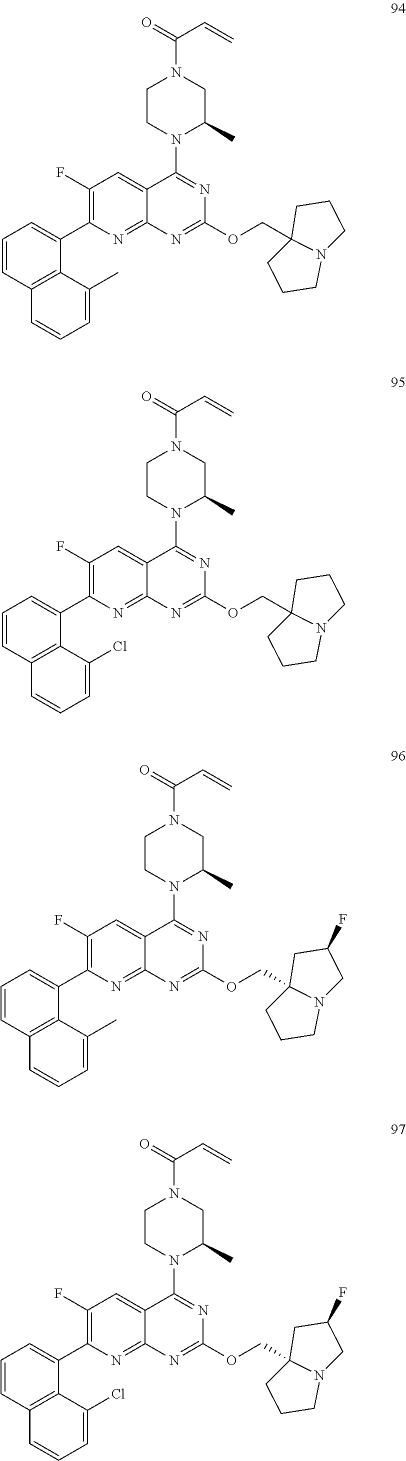 [SMILES: C=CC(=O)N1CCN(c2nc(OCC34CCCN3CCC4)nc3nc(-c4cccc5cccc(C)c45)c(F)cc23)[C@H](C)C1 ; C=CC(=O)N1CCN(c2nc(OCC34CCCN3CCC4)nc3nc(-c4cccc5cccc(Cl)c45)c(F)cc23)[C@H](C)C1 ; C=CC(=O)N1CCN(c2nc(OC[C@@]34CCCN3C[C@H](F)C4)nc3nc(-c4cccc5cccc(C)c45)c(F)cc23)[C@H](C)C1 ; C=CC(=O)N1CCN(c2nc(OC[C@@]34CCCN3C[C@H](F)C4)nc3nc(-c4cccc5cccc(Cl)c45)c(F)cc23)[C@H](C)C1]

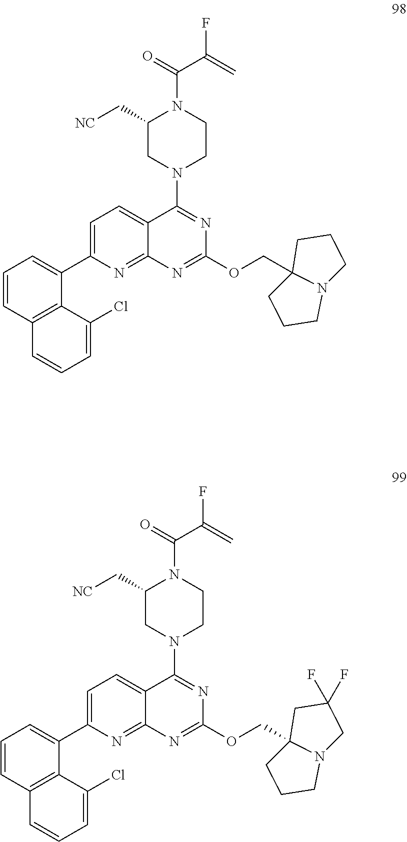 [SMILES: C=C(F)C(=O)N1CCN(c2nc(OCC34CCCN3CCC4)nc3nc(-c4cccc5cccc(Cl)c45)ccc23)C[C@@H]1CC#N ; C=C(F)C(=O)N1CCN(c2nc(OC[C@@]34CCCN3CC(F)(F)C4)nc3nc(-c4cccc5cccc(Cl)c45)ccc23)C[C@@H]1CC#N]

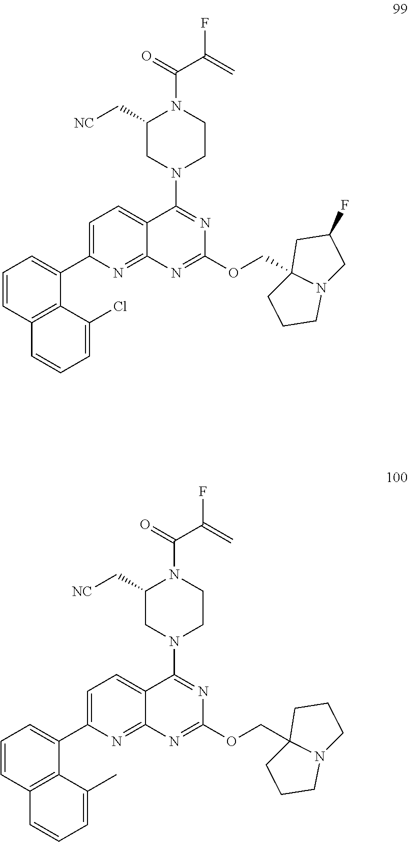 [SMILES: C=C(F)C(=O)N1CCN(c2nc(OC[C@@]34CCCN3C[C@H](F)C4)nc3nc(-c4cccc5cccc(Cl)c45)ccc23)C[C@@H]1CC#N ; C=C(F)C(=O)N1CCN(c2nc(OCC34CCCN3CCC4)nc3nc(-c4cccc5cccc(C)c45)ccc23)C[C@@H]1CC#N]

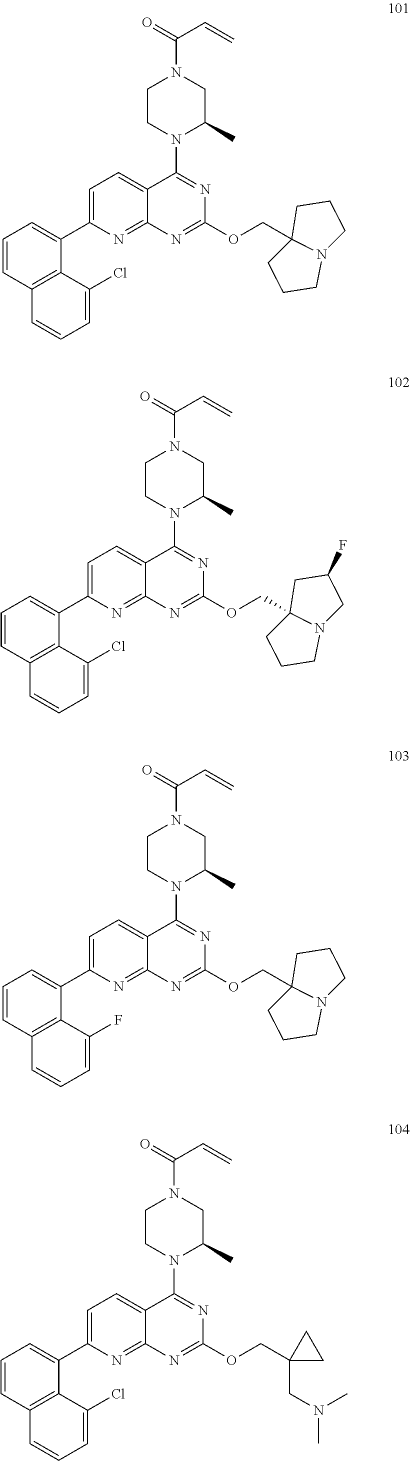 [SMILES: C=CC(=O)N1CCN(c2nc(OCC34CCCN3CCC4)nc3nc(-c4cccc5cccc(Cl)c45)ccc23)[C@H](C)C1 ; C=CC(=O)N1CCN(c2nc(OC[C@@]34CCCN3C[C@H](F)C4)nc3nc(-c4cccc5cccc(Cl)c45)ccc23)[C@H](C)C1 ; C=CC(=O)N1CCN(c2nc(OCC34CCCN3CCC4)nc3nc(-c4cccc5cccc(F)c45)ccc23)[C@H](C)C1 ; C=CC(=O)N1CCN(c2nc(OCC3(CN(C)C)CC3)nc3nc(-c4cccc5cccc(Cl)c45)ccc23)[C@H](C)C1]

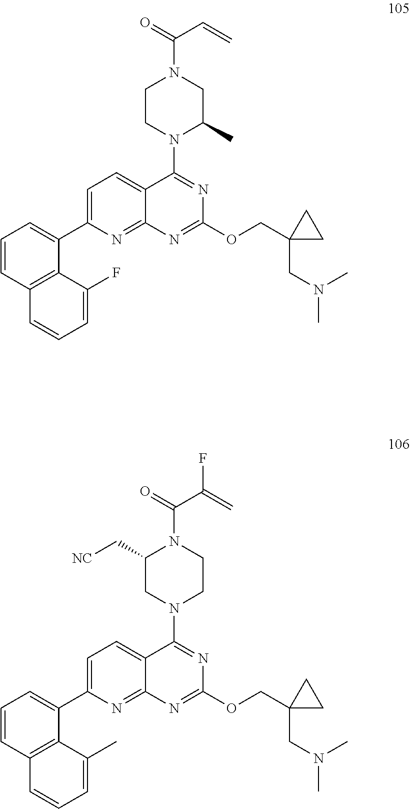 [SMILES: C=CC(=O)N1CCN(c2nc(OCC3(CN(C)C)CC3)nc3nc(-c4cccc5cccc(F)c45)ccc23)[C@H](C)C1 ; C=C(F)C(=O)N1CCN(c2nc(OCC3(CN(C)C)CC3)nc3nc(-c4cccc5cccc(C)c45)ccc23)C[C@@H]1CC#N]

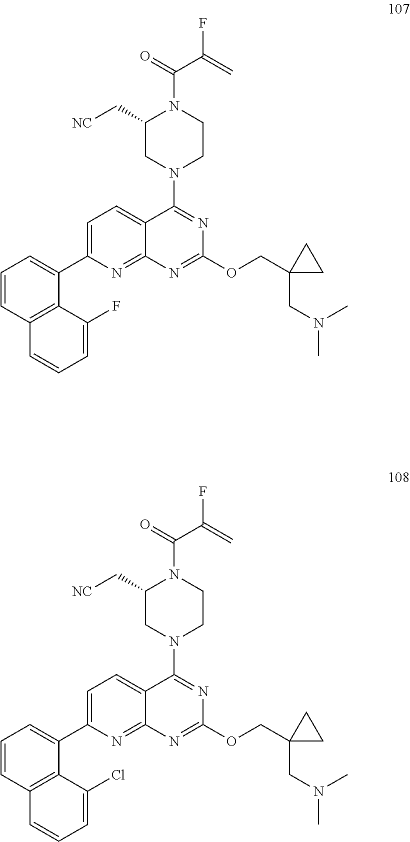 [SMILES: C=C(F)C(=O)N1CCN(c2nc(OCC3(CN(C)C)CC3)nc3nc(-c4cccc5cccc(F)c45)ccc23)C[C@@H]1CC#N ; C=C(F)C(=O)N1CCN(c2nc(OCC3(CN(C)C)CC3)nc3nc(-c4cccc5cccc(Cl)c45)ccc23)C[C@@H]1CC#N]

### EXAMPLE 94 Biological Activity Test

The present invention is further described and explained below in conjunction with test examples, but these implementations are not meant to limit the scope of the present invention.

### 1. Tumor Cell Proliferation Inhibition Assay

### 1 experimental method

The H358 (KRAS G¹²C mutant) cells were digested, centrifuged and resuspended, and the cell density was measured with a Scepter automatic cell counter. The cells were diluted to a solution containing 44,000 cells per milliliter, and the density-adjusted cell solution was 90 microliters per well. into a 96-well culture plate. The 96-well plate was placed in a 37° C., 5% CO2 incubator. After the cells were cultured for 24 hours, different concentrations of the compounds to be tested were added. The cells were incubated with the compounds for 72 hours in the presence of 10% fetal bovine serum. Glo Luminescent Cell Viability Assay Kit (see manufacturer's instructions for details) was used to measure ATP content to assess cell growth inhibition. Briefly, 30 μl of Cell Titer-Glo reagent was added to each well, and the plate was shaken for 10 minutes to induce cell lysis. Fluoroskan

Ascent FL (Thermo) assay was used to record the fluorescent signal, and the maximum signal value was obtained from cells treated with dimethyl sulfoxide for 72 hours. The minimum signal value was obtained from the medium alone (the number of cells was zero), and the % inhibition rate=(the maximum signal value compound signal value)/(the maximum signal value—the minimum signal value×100%, and the data were processed using Graphpadprism 5 software. By sigmoid Dose-response curve fitting to calculate the IC50 value. “A” means IC50≤50 nM; “B” means 50<IC50≤500 nM; “C” means 500<IC50≤2000 nM; “D” means 2000 nM<IC50

### 2Experimental Results

Calculate the IC₅₀ of each compound in the above experiment, and the results are shown in Table 1 below

TABLE 1
IC₅₀(nm) of inhibitory activity of compounds
on tumor cell proliferation.

| | NCI-H538 |
|---|---|
| No | IC₅₀(nm) |
| 1 | D |
| 2 | D |
| 3 | D |
| 4 | D |
| 5 | A |
| 6 | A |
| 7 | A |
| 8 | A |
| 9 | A |
| 10 | A |
| 11 | D |
| 12 | C |
| 13 | A |
| 14 | A |
| 15 | A |
| 16 | A |
| 17 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | A |
| 91 | A |
| 92 | A |
| 93 | A |

### 2. KRAS-G2C/SOS1 Binding Experiment

### 1 Experimental Method

A) Dilute Tag2-KRAS-G12C protein and Tag1-SOS protein with diluent at 1:100, anti-Tag1-Tb3+ antibody and anti-Tag2-XL665 with detection buffer at 1:100 or 1:25 respectively Dilute.
B) Dilute the compound to be tested with diluent starting from 10000 mM concentration, 4 times gradient, a total of 6 concentration gradients, and dilute to 10× stock solution.
C) In a 96-well plate, add 4 uLTag2-KRAS-G12C protein, 4 uL Tagl-SOS protein, 2 ul dilution solution (positive control) or test compound (different concentrations of 10× stock solution) to each well in sequence, a total of 10 ul, and incubate at room temperature for 15 10 minutes later, add pre-mixed 5 uL anti-Tagl-Tb3+ and 5 uL anti-Tag2-xL665, seal the plate, incubate at room temperature for 2 hours, and read the HTRF signal with a TECAN INFINITEF NANO+ microplate reader.

### 2 Experimental Results

The ratio of love body and donor excitation signals for each single well was calculated using the formula ratio=665 nm signal value/620 nm signal value×104. Data were processed using Graphpadprism5 software. IC₅₀ values were calculated by sigmoidal dose-response curve fitting. “+” means IC₅₀<50 nM; “++” means 50<IC50≤500nM; “+++” means 500 nM<IC₅₀, the results are shown in Table 2 below

TABLE 2
IC₅₀(nm) of compounds inhibiting
KRAS-G2C/SOS1 binding activity

| | KRAS-G2C/SOS1 |
|---|---|
| | binding |
| No | IC₅₀(nm) |
| 1 | +++ |
| 2 | +++ |
| 3 | +++ |
| 4 | +++ |
| 5 | + |
| 7 | + |
| 28 | + |

Although the present invention has been described in detail above, it will be understood by those skilled in the art that various modifications and variations can be made in the present invention without departing from the spirit and scope of the invention. The scope of the rights of the present invention is not limited to the detailed description above, but should be attributed to the claims.

1. A compound with general formula (I), its stereoisomer, pharmaceutically acceptable salt, polymorph or isomer. The structure of the compound shown in general formula (I) is as follows: 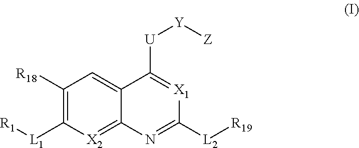
Wherein,
Each L₁ is independently selected from bond, O, NH, CH₂, CO or S at each occurrence;
Each R₁ at each occurrence is independently selected from phenyl, naphthyl, 5-membered heteroaryl, 6-membered heteroaryl, 7-membered heteroaryl, 8-membered heteroaryl, 9-membered heteroaryl, or 10-membered heteroaryl, each heteroaryl at each occurrence independently containing 1, 2, 3 or 4 heteroatoms selected from N, O, or S; each R₁ at each occurrence independently and optionally substituted or unsubstituted with 1, 2, 3, 4, 5 or 6 R₂₀;
Each R₂₀ at each occurrence is independently selected from deuterium, halogen, oxo, —C₁₋₆ alkyl, —C₁₋₆ alkylene-(halogen)₁₋₃, C₁₋₆ hetero alkyl, —CN, —OR₆, —C₁₋₆ alkylene-(OR₆)₁₋₃, —O—C₁₋₆ alkylene-(halogen)₁₋₃, —SR₆, —S—C₁₋₆ alkylene-(Halogen)₁₋₃, —NR₆R₇, —C₁₋₆ alkylene —NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl; each R₁₂ is independently and optionally substituted or unsubstituted with 1, 2, 3, 4, 5 or 6 Substituents selected from deuterium, halogen, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —NR₆ C(═O)R₇ or —S(O)₂NR₆R₇;
Each X₁, X₂ is independently selected from N, CR₂₁ at each occurrence;
Each R₁₈ is independently selected from H, D, cyano, halogen, C₁₋₆ alkyl, COOH, NHCOH, CONH₂, OH or —NH₂;
Each R₂₁ is independently selected from H, D, cyano, halogen, C₁₋₆ alkyl, COOH, NHCOH, CONH₂, OH or —NH₂;
Each L₂ at each occurrence is independently selected from bond, O, NH, C₁₋₆ alkyl, CO, OC₁₋₆ alkyl, NHC₁₋₆ alkyl or S at each occurrence;
Each R₁₉ is independently selected from 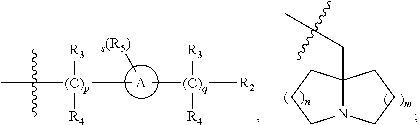
Each Ring A is a C₃₋₁₀ carbocyclic ring, 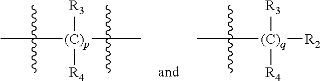
can be attached to the same or to a different atom of ring A;
Each R₂ is —OR₆, —NR₆R₇, —SR₆, —S(═O)R₆, —S(═O)₂R₆, 5-10 membered heteroaryl or 3-10 membered heterocyclyl, each heterocyclyl and heteroaryl at each occurrence independently containing 1, 2, 3 or 4 heteroatoms selected from N, O, S, S═O or S(═O)₂. Each R₂ at each occurrence independently and optionally substituted or unsubstituted with 1, 2, 3, 4, 5 or 6 R₂₂;
Each R₃ and R₄ at each occurrence is independently selected from deuterium, hydrogen, halogen, —C₁₋₆ alkyl, —C₂₋₆ alkenyl, —C₂₋₆ alkynyl, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇ or —C₃₋₁₀ carbocyclyl, each heterocyclyl and heteroaryl at each occurrence is independently containing 1, 2, 3 or 4 heteroatoms selected from N, O, S, S═O or S(═O)₂, each R₃ and R₄ at each occurrence is independently and optionally substituted or unsubstituted with 1, 2, 3, 4, 5 or 6 subsituents selected from deuterium, halogen, oxo, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —NR₆ C(═O)R₇ or —S(O)₂NR₆R₇.
Each R₅ at each occurrence is independently selected from deuterium, halogen, oxo, —C₁₋₆ alkyl, —C₁₋₆ alkylene-(halogen)₁₋₃, C₁₋₆ heteroalkyl, —CN, —OR₆, —C₁₋₆ alkylene-(OR₆)₁₋₃, —O—C₁₋₆ alkylene-(halogen)₁₋₃, —SR₆, —S—C₁₋₆ alkylene-(Halogen)₁₋₃, —NR₆R₇, —C₁₋₆ alkylene —NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆ carbocyclyl, each heterocyclyl and heteroaryl at each occurrence independently contains 1, 2, 3 or 4 heteroatoms selected from N, O, S, S═O or S(═O)₂; each R₃ and R₄ at each occurrence independently and optionally substituted or unsubstituted with 1, 2, 3, 4, 5 or 6 substituents selected from deuterium, halogen, oxo, —C₁₋₆ alkyl, —C₁₋₆ alkoxy, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇;
Each R₆ and R₇ at each occurrence is independently selected from hydrogen or —C₁₋₆ alkyl, each R₆ and R₇ is independently and optionally substituted or unsubstituted with 1, 2, 3, 4, 5 or 6 R₂₂ or R₇ and R₇ together with the N atom to which they are connected together form a 3-10-membered heterocycle, and the 3-10-membered heterocycle may further comprise 1, 2, 3 or 4 heteroatoms selected from N, O, S, S(═O) or S(═O)₂, and the 3-10 membered heterocycle is independently and optionally substituted or unsubstituted by 1, 2, 3, 4, 5 or 6 R₂₂;
Each R₂₂ at each occurrence is independently selected from deuterium, halogen, oxo, —C₁₋₆ alkyl, —C₁₋₆ alkylene-(halogen)₁₋₃, C₁₋₆hetero alkyl, —CN , —C₁₋₆ alkyl, —C₁₋₆ alkylene-(O—C₁₋₆alky)₁₋₃, —O—C₁₋₆ alkylene-(halogen)₁₋₃, —S—C₁₋₆ alkyl, —S—C₁₋₆ alkylene-(halogen)₁₋₃, N—C₁₋₆alkyl-C₁₋₆alkyl, —C₁₋₆alkylene-NC₁₋₆alkylC₁₋₆alkyl,—C(═O)C₁₋₆ alkyl, —C(═O)OC₁₋₆ alkyl, —OC(═O)C₁₋₆alkyl, —C(═O)NC₁₋₆ alkylC₁₋₆ alkyl, —NC₁₋₆alkylC(═O)C₁₋₆alkyl,—S(O)₂NC₁₋₆alkyl, C₁₋₆alkyl or —C₃₋₆ carboncyclyl;
s is selected from 0, 1, 2, 3, 4, 5 or 6;
p is selected from 0, 1, 2, 3, 4, 5 or 6;
q is selected from 0, 1, 2, 3, 4, 5 or 6.
m is selected from 1, 2, 3
n is selected from 1, 2, 3
U is independently selected from —C₀₋₄ alkyl-, —CR₈R₉—, —C₁₋₂ alkyl(R₈)(OH)—, —C(O)—, —CR₈R₉₀—, —OCR₈R₉—, —SCR₈R₉—, —CR₈R₉S—, —NR₈—, —NR₈C(O)—, —C(O)NR₈—, —NR₈C(O)NR₉—, —CF₂—, —O—, —S—, —S(O)ₘ—, —NR₈S(O)ₘ—, —S(O)ₘNR₈—;
Y is absent or selects from C₃₋₈ cycloalkyl, 3-8 membered heterocycloalkyl, 5-12 membered fused alkyl, 5-12 membered fused heterocyclyl, 5-12 membered spirocyclyl, 5-12 membered spiroheterocyclyl, aryl or heteroaryl, wherein cycloalkyl, heterocycloalkyl, spirocyclyl, fused cyclyl fused heterocyclyl, spiroheterocyclyl, aryl or heteroaryl is optionally replaced by one or more G¹;
Z is independently selected from cyano, —NR₁₀CN, 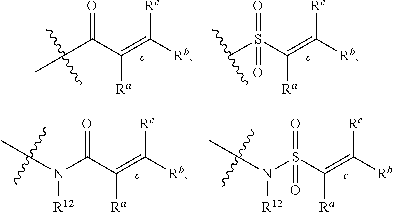
The bond c is a double bond or a triple bond;
When c is a double bond, Rₐ, R_{b} and R_{c} are each independently selected from H, deuterium, cyano, halogen, C₁₋₆ alkyl, C₃₋₆ cycloalkyl or 3-6 membered heterocyclyl. wherein the alkyl, cycloalkyl and heterocyclyl are optionally substituted with one or more G²s;
R^{a} and R^{b} or R^{b} and R^{c} optionally together with the carbon atoms to which they are attached form a 3-6 membered ring optionally containing heteroatoms;
When the bond c is a triple bond, Rₐ and R_{c} are absent, and R_{b} independently selected from H, deuterium, cyano, halogen, C₁₋₆ alkyl, C₃₋₆ cycloalkyl or 3-6 membered heterocyclyl is substituted with one or more G³s;
R₁₀ is independently selected from H, deuterium, C₁₋₆ alkyl, C₃₋₆ cycloalkyl or 3-6 membered heterocyclyl, wherein the alkyl, cycloalkyl and heterocyclyl are optionally substituted with one or more G⁴s;
G¹, G², G³ and G⁴ are each independently selected from deuterium, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR₁₁, —OC(O)NR₁₁R₁₂, —C(O)OR₁, —C(O)NR₁₁R₁₂, —C(O)R₁₁R₁₂, —NR₁₁C(O)R₁₂, —NR₁₁C(O)NR₁₂R₁₃, —S(O)ₘR₁₁ or —NR₁₁S(O)ₘR₁₂, wherein the alkyl, alkenyl, alkynyl, cycloalkyl, heterocycloalkyl, aryl, Heteroaryl is optionally replaced by 1 or more deuterium, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR₁₄, —OC(O)NR₁₄R₁₅, —C(O)OR₁₄, —C(O)NR₁₄R₁₅, —C(O)R₁₄, —NR₁₄R₁₅, —NR₁₄C(O)R₁₅, —NR₁₄C(O)NR₁₅R₁₆, —S(O)ₘR₁₄ or —NR₁₄S(O)nR₁₅ substituent;
R₈, R₉, R₁₁, R₁₂, R₁₃, R₁₄ and R₁₅ is independently selected from Hydrogen, deuterium, cyano, halogen, C₁₋₆ alkyl, C₃₋₈ cycloalkyl or 3-8 membered monocyclic heterocyclyl, monocyclic heteroaryl or phenyl;
and m is 1 or 2.

2. The compounds of formula (I), a pharmaceutically acceptable salt thereof, or a stereoisomer thereof according to claim 1, wherein each ring A is a 3-membered carbocyclyl, 4-membered carbocyclyl, 5-membered carbocyclyl, or 6-membered carbocyclyl. The 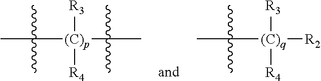
may be attached to the same carbon atom or a different atom of ring A. Each R₂ at a occurrence is independently selected from —NR₆R₇ or 3-6 membered heterocyclyl. Each heterocyclyl at each occurrence independently contains 1 heteroatoms selected from N. Each R₂ at each occurrence is independently and optionally substituted or unsubstituted with 1, 2, 3, 4, 5 or 6 R₂₀;
R₆ and R₇ in each R₂ are independently selected from hydrogen, deuterium, methyl, ethyl, propyl or isopropyl at each occurrence; or R₆ and R₇ in R₂ together with the N atoms to which they ar attached together form a 3-6-membered heterocycle, the 3-6- membered heterocycle may further comprise 1 heteroatom selected from N, and the 3-6-ary heteroring is independently and selectively replaced or not replaced by 1, 2, 3, 4, 5 or 6 R₂₀;
Preferably, each R₂ at each occurrence is independently selected from —NH₂, —N(CH₃)₂, —N(CH₃)(CH₂CH₃), —N(CH₂CH₃)₂, 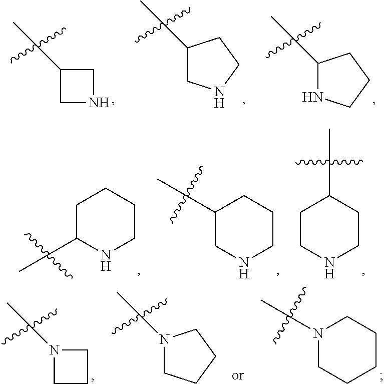
Each R₂ is independently and optionally replaced by 1, 2, 3, 4, 5 or 6 R₂₀ or not;
More preferably, each R₂ at each occurrence is independently selected from —NH₂, —N(CH₃)₂, —N(CH₃)(CH₂CH₃),—N(CH₂CH₃)₂, 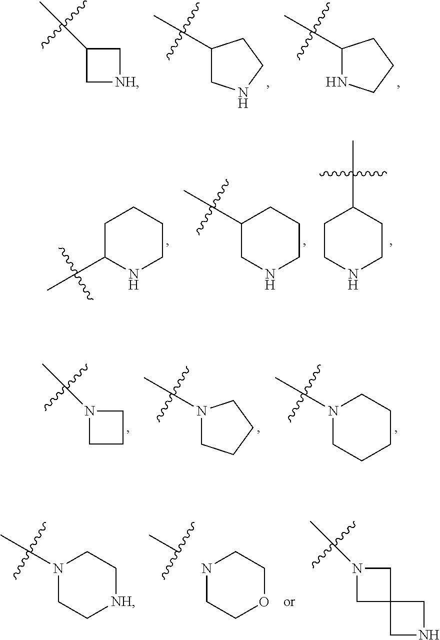
Each R₂ is independently and optionally substituted or unsubstituted with 1, 2, 3, 4, 5 or 6 R₂₀ at each occurrence;
Preferably, each R₂₀ at each occurrence is independently selected from deuterium, halogen, oxo, —C₁₋₆ alkyl, C₁₋₆ alkenyl, —C₁₋₆ alkynyl, —C₁₋₆ alkylene-(halogen)₁₋₃, C₁₋₆heteroalkyl, —CN, —OR₆, —C₁₋₆ alkylene-(OR₆)₁₋₃, —O—C₁₋₆ alkylene-(halogen)₁₋₃, —SR₆, —S—C₁₋₆ alkylene(halogen)₁₋₃, —NR₆R₇, —C₁₋₆ alkylene —NR₆R₇, —C(═O)R₆, —C(═O)OR₆, —OC(═O)R₆, —C(═O)NR₆R₇, —NR₆C(═O)R₇, —S(O)₂NR₆R₇ or —C₃₋₆carboncyclyl; Each R₁₂ independently and optionally substituted or unsubstituted with 1, 2, 3, 4, 5 or 6 substituents selected from deuterium, halogen, oxo, —C₁₋₆alkyl, —C₁₋₆ alkoxyl, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇.
More preferably, each R₂₀ at each occurrence is independently selected from -deuterium, —F, —Cl, —Br, oxo, methyl, ethyl, propyl, isopropyl, —CH═CH₂, —CH═CHCH₃, —CH₂CH═CH₂, —CH₂═CHCH₂CH₃, —C≡CH, —C≡CCH₃, —CH₂C≡CH, —CH₂F, —CHF₂, —CF₃, —CH₂CH₂F, —CH₂CHF₂, —CH₂CF₃, —CH₂CH₂CH₂F, —CH₂CH₂CH₂F₂, —CH₂CH₂CF₃, —CH₂OCH₃, —CH2CH₂OCH₃, —CH₂CH₂CH₂OCH₃, —CN , —OH , —OCH₃, —OCH₂CH₃, —OCH₂CH₂CH₃, —OCH(CH₃)₂, —CH₂OH , —CH₂CH₂OH , —CH₂CH₂CH₂OH , —OCH₂F, —OCHF₂, —OCF ₃, —OOOF, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SOF, —SCHF₂, —SCF₃, —SCH₂CH₂F, —SCH₂CH₂F₂, —SCH₂CF₃, —SCH₂CH₂CH₂F, —SCH₂CH₂CHF₂, —SCH₂CH₂CF₃, —NH ₂, —NHCH₃, —NHCH₂CH₃, —NHCH₂CH₂CH₃, —NHCH(CH₃)₂, —N(CH₃)₂, —N(CH₃)CH₂CH₃, —N(O)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3-membered carbocyclyl, 4-membered carbocyclyl, 5-membered carbocyclyl, or 6-membered carbocyclyl membered carbocyclyl; each R₂₀ is independently and optionally substituted or unsubstituted with 1,2,3,4,5 or 6 substituents selected from -deuterium, —F, —Cl, —Br, methyl, ethyl, propyl, isopropyl, methoxy, ethoxy, propoxy, isopropoxy, oxo, —OH, —NH ₂, —NHCH₃, —N(CH₃)₂, —CN, —C(═O)CH₃, —C(═O)OO, —OC(═O)O, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂.

3. The compound of formula (I), a pharmaceutically acceptable salt thereof, or a stereoisomer thereof according to claim 1, wherein Each R₃ and R₄ are independently selected from deuterium, hydrogen, halogens, —C₁₋₆ alkyl, —C₂₋₆ alkenyl, —C₂₋₆ alkynyl, oxo, —OR₆, —NR₆R₇, —CN, —C(═O)R₆, —C(═O)OR₆, —OC(═O)R₆, —C(═O)NR₆R₇, —NR₆C(═O)R₇ or —S(O)₂NR₆R₇ or —C₃₋₁₀ carboncyclyl. Each heterocycly and heteroaryl independently contains 1, 2, 3, or 4 heteroatoms selected from N, O, S, S═O, or S(═O)₂ at each occurrence; Each R₃ and R₄ is independently and optionally substituted or not substituted by 1, 2, 3, 4, 5 or 6 substitutes selected from deuterium, halogens, oxo, —C₁₋₆ alkyl, —C₁₋₆ alkoxy, oxo, —OR₆, —NR₆R₇, —CN, —C (═O) R₆, —C (═O) OR₆, —OC (═O)R₆, —C(═O) NR₆R₇, —NR₆C (═O) R₇ or —S (O)₂NR₆R₇;
R₆ and R₇ in each R₃ and R₄ are independently selected from hydrogen, deuterium, or —C₁₋₃ alkyl at each occurrence;
Preferably, each R₃ and R₄ at each occurance is independently selected from hydrogen, —F, —Cl, —Br, methyl, ethyl, propyl, isopropyl, vinyl, propyl, isopropenyl, ethynyl, propynyl, oxy, —OH, —OCH₃, —OCH₂CH₃, —OCH₂CH₂CH₃, —OCH(CH₃) ₂, —NH ₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CN, —C(═O)CH₃, —C(═O)OCH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3-membered carbocyclyl, 4-membered carbocyclyl, 5-membered carbocyclyl or 6-membered carbocyclyl; Each R₃ or R₄ is independently and optionally substituted or unsubstituted with 1, 2, 3, 4, 5 or 6 substituents selected from —F, —Cl, —Br, oxo, methyl, ethyl, propyl, isopropyl, —OH, OCH₃, —OCH₂CH₃, —OCH₂CH₂CH₃, —OCH(CH₃)₂, —NH₂, —N(CH₃)₂, —CN, —C(═O)CH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂.

4. The compound of formula (I), a pharmaceutically acceptable salt thereof, or a stereoisomer thereof according to claim 1, wherein each R₅ at each occurrence is independently selected from deuterium, —F, —Cl, —Br, —C₁₋₃ alkyl,—C₁₋₃ alkylene-(halogen)₁₋₃, C₁₋₃ heteralkyl, —C₂₋₃enyle, —C₂₋₃yne,—CN, —OR₆, —C₁₋₆ alkyne-(OR₆)₁₋₃, —O—C₁₋₆ alkylene-(halogen)₁₋₃, —SR₆, —S—C₁₋₆ alkylene-(halogen)₁₋₃, —NR₆R₇, —C₁₋₆ alkylene-NR₆R₇, —C(═O)R₆, —C(═O OR₆, —OC(═O)R₆, —C(═O)NR₆R₇, —NR₆ C(═O)R₇, —S(O)₂NR₆R₇ or —C₃₋₆ carbocyclyl, each heterocyclic group and heteroaryl group independently contains 1, 2, 3 or 4 heteroatoms selected from N, O, S, S═O or S(═O)₂ at each occurrence; Each R₅ is independently and optionally substituted or not substituted by 1, 2, 3 or 4, 5 or 6 substituents selected from deuterium, —F, —Cl, —Br, oxo, —C₁₋₆ alkyl, —C₁₋₆ alkoxy, oxo, —OR₆, —NR₆R₇, —CN, —C (═O) R₆, —C (═O) OR₆, —OC (═O) R₆, —C (═O) NR₆R₇, —NR₆C (═O)R₇ or —S(O)₂NR₆R₇;
Each R₆ and R₇ in each of R₅ are independently selected at each occurrence from hydrogen, deuterium or —C₁₋₃ alkyl; or R₆ and R₇ in R₅ together with the N atom to which they are commonly attached form a 3-6 membered heterocycle, and the 3-6 membered heterocycle may further comprise 1 heteroatom selected from N, and the 3-6 membered heterocycle is independently optionally selected from N, O or 1, 2, 3, and 4. heteroatom of S;
Preferably, each R₅ is independently at each occurrence selected from deuterium, —F, —Cl, —Br, methyl, ethyl, propyl, isopropyl, vinyl, propenyl, isopropenyl, acetylene base, propynyl, -methylene-(halogen)₁₋₃, -ethylene-(halogen)₁₋₃, -propylene-(halogen)₁₋₃, heteromethyl, heteroethyl, Heteropropyl, vinyl, propenyl, ethynyl, propynyl, oxo, —OR₆, -methylene-(OR₆)₁₋₃, -ethylene-(OR₆)₁₋₃, -propylene Base-(OR₆)₁₋₃, —O-methylene-(halogen)₁₋₃, —O-ethylene-(halogen)₁₋₃, —O-propylene-(halogen)₁₋₃, —NR₆R₇, -methylene-NR₆R₇, -ethylene-NR₆R_{7,}-propylene-NR₆R₇, —CN, —C(═O)R₆, —C(═O)OR₆, —OC(═O) R₆, —C(═O)NR₆R₇, —NR₆C(═O)R₇, —S(O)₂NR₆R₇, phenyl, naphthyl, 5-membered heteroaryl, 6-membered heteroaryl, 7-membered heteroaryl, 6-membered heteroaryl, 8-membered heteroaryl, 10-membered heteroaryl, 3-membered heterocyclyl, 4-membered heterocyclyl, 5-membered heterocyclyl, 6-membered heterocyclyl, 3-membered carbocyclyl, 4-membered carbocyclyl, 5-membered carbocyclyl, or 6-membered carbocyclyl, each heterocyclyl and heteroaryl independently at each occurrence containing 1, 2, 3, or 4 heteroatoms selected from N, O, or S ; each R₇ at each occurrence is independently and optionally substituted or unsubstituted with 1, 2, 3, 4, 5 or 6 substituents selected from —F, —Cl, —Br, oxo, methyl, ethyl, propyl, Isopropyl, —OR₆, —NR₆R₇, —CN, —C(═O)R₆, —C(═O)OR₆, —OC(═O)R₆, —C(═O)NR₆R₇, —NR₆C(═O)R₆, or —S(O)₂NR₆R₇. R₆ and R₇ in each R₅ are independently selected from hydrogen, deuterium, methyl, ethyl, propyl, isopropyl at each occurrence; Or or the N atom to which they are commonly attached Together to form 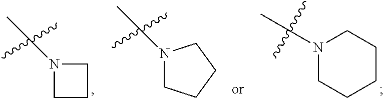 [SMILES: *N1CCC1 ; *N1CCCC1 ; *N1CCCCC1]
More preferably, each R₅ at each occurrence is independently selected from deuterium, —F, —Cl, —Br, methyl, ethyl, propyl, isopropyl, —CH₂F, —CHF₂, —CF₃, —CH₂CH₂F, —CH₂CHF₂, —CH₂CF ₃, —CH₂CH₂CH₂F, —CH₂CH₂CH₂F ₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —OCH₂F, —OCHF₂, —OCF₃, —OOOF, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SOF, —SCHF₂, —SCF₃, —SCH₂CH₂F, —SCH₂CH₂F₂, —SCH₂CF₃, —SCH₂CH₂CH₂F, —SCH₂CH₂CHF₂, —SCH₂CH₂CF₃, —NH₂, —NHCH₃, —NHCH₂CH₃, —NHCH₂CH₂CH₃, —NHCH(CH₃)₂, —N(CH₃)₂, —N(CH₃)CH₂CH₃, —N(O)CH₂CH₂CH₃, —N(CH₃)CH(CH₃)₂, —CH₂NH₂, —CH₂CH₂NH ₂, —CH₂CH₂CH₂NH ₂, —CH₂N(CH₃)₂, —CH₂CH₂N(CH₃)₂, —CH₂CH₂CH₂N(CH₃)₂, —C(═O)CH₃, —C(—O)OCH₃, —C(═O)OCH₂CH₃, —C(═O)OCH₂CH₂CH₃, —OC(—O)CH₃, —C(50 O)NH₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃), —S(O)₂N(CH₃)₂, 3-membered carbocyclyl, 4-membered carbocyclyl, 5-membered carbocyclyl, or 6-membered carbocyclyl; each R₂₀ is independently and optionally substituted or unsubstituted with 1, 2, 3, 4, 5 or 6 substituents selected from -deuterium, —F, —Cl, —Br, methyl, ethyl, propyl, isopropyl, Methoxy, ethoxy, propoxy, isopropoxy, oxo, —OH, —NH₂, —NHCH₃, —N(CH₃)₂, —CN, —C(═O)CH₃, —C(═O)OO, —OC(═O)O, —C(═O)NF₁₂, —C(═O)NH(CH₃), —C(═O)N(CH₃)₂, —NHC(═O)CH₃, —N(CH₃)C(═O)CH₃, —S(O)₂NH₂, —S(O)₂NH(CH₃) or —S(O)₂N(CH₃)₂.

5. The compounds of formula (I), a pharmaceutically acceptable salt thereof, or a stereoisomer thereof according to claim 1, wherein 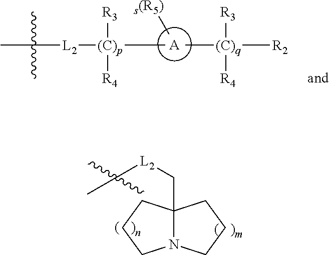
are selected from

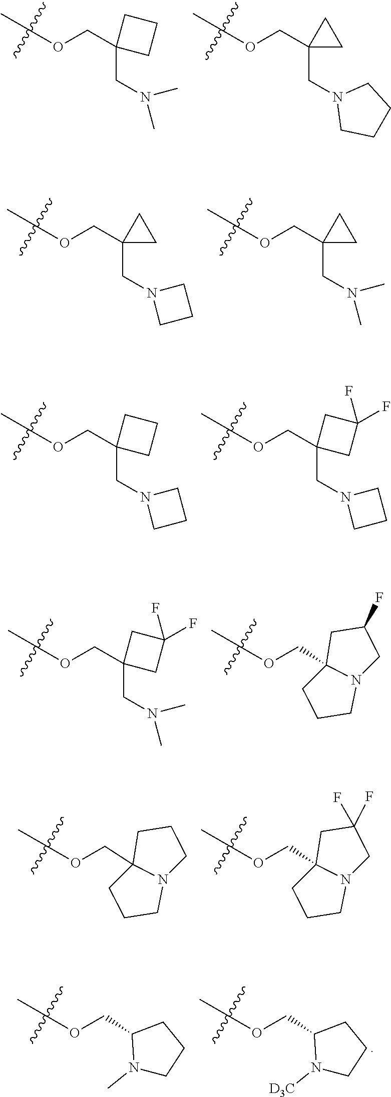

6. The compound of formula (I) according to claim 1, its pharmaceutically acceptable salt or its stereoisomer, wherein, the compound is selected from the following structures:

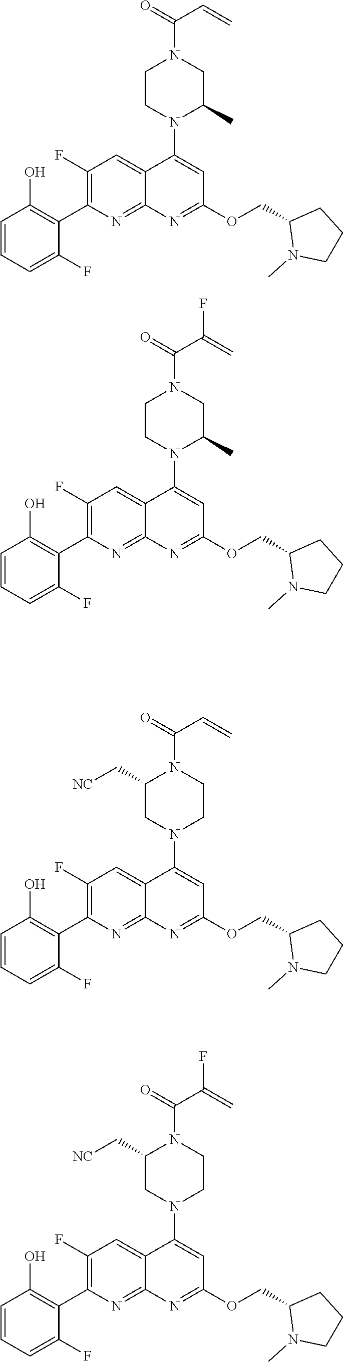 [SMILES: C=CC(=O)N1CCN(c2cc(OC[C@@H]3CCCN3C)nc3nc(-c4c(O)cccc4F)c(F)cc23)[C@H](C)C1 ; C=C(F)C(=O)N1CCN(c2cc(OC[C@@H]3CCCN3C)nc3nc(-c4c(O)cccc4F)c(F)cc23)[C@H](C)C1 ; C=CC(=O)N1CCN(c2cc(OC[C@@H]3CCCN3C)nc3nc(-c4c(O)cccc4F)c(F)cc23)C[C@@H]1CC#N ; C=C(F)C(=O)N1CCN(c2cc(OC[C@@H]3CCCN3C)nc3nc(-c4c(O)cccc4F)c(F)cc23)C[C@@H]1CC#N]

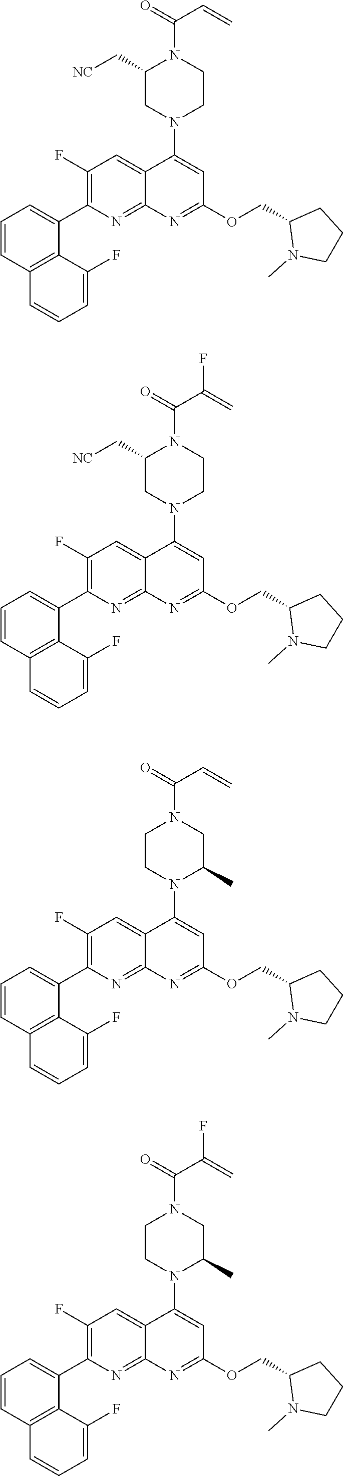 [SMILES: C=CC(=O)N1CCN(c2cc(OC[C@@H]3CCCN3C)nc3nc(-c4cccc5cccc(F)c45)c(F)cc23)C[C@@H]1CC#N ; C=C(F)C(=O)N1CCN(c2cc(OC[C@@H]3CCCN3C)nc3nc(-c4cccc5cccc(F)c45)c(F)cc23)C[C@@H]1CC#N ; C=CC(=O)N1CCN(c2cc(OC[C@@H]3CCCN3C)nc3nc(-c4cccc5cccc(F)c45)c(F)cc23)[C@H](C)C1 ; C=C(F)C(=O)N1CCN(c2cc(OC[C@@H]3CCCN3C)nc3nc(-c4cccc5cccc(F)c45)c(F)cc23)[C@H](C)C1]

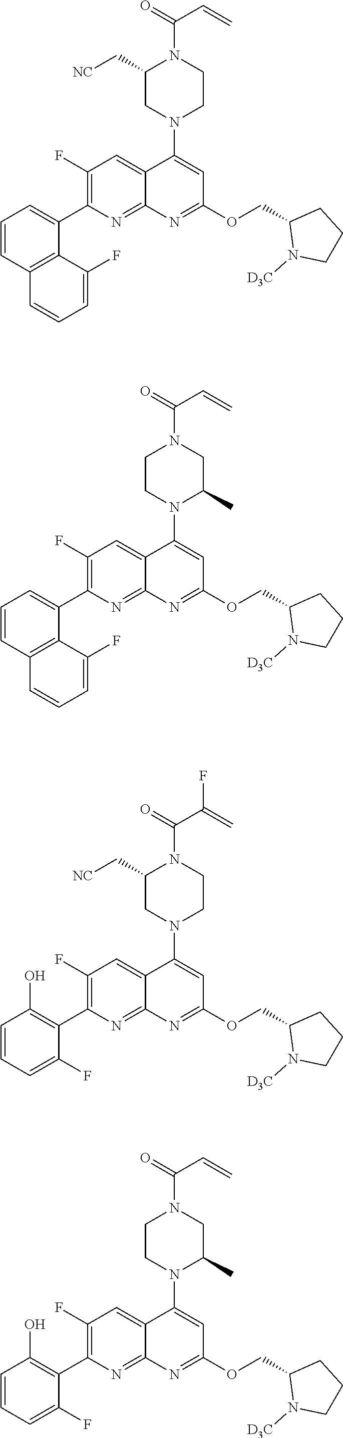 [SMILES: [2H]C([2H])([2H])N1CCC[C@H]1COc1cc(N2CCN(C(=O)C=C)[C@@H](CC#N)C2)c2cc(F)c(-c3cccc4cccc(F)c34)nc2n1 ; [2H]C([2H])([2H])N1CCC[C@H]1COc1cc(N2CCN(C(=O)C=C)C[C@H]2C)c2cc(F)c(-c3cccc4cccc(F)c34)nc2n1 ; [2H]C([2H])([2H])N1CCC[C@H]1COc1cc(N2CCN(C(=O)C(=C)F)[C@@H](CC#N)C2)c2cc(F)c(-c3c(O)cccc3F)nc2n1 ; [2H]C([2H])([2H])N1CCC[C@H]1COc1cc(N2CCN(C(=O)C=C)C[C@H]2C)c2cc(F)c(-c3c(O)cccc3F)nc2n1]

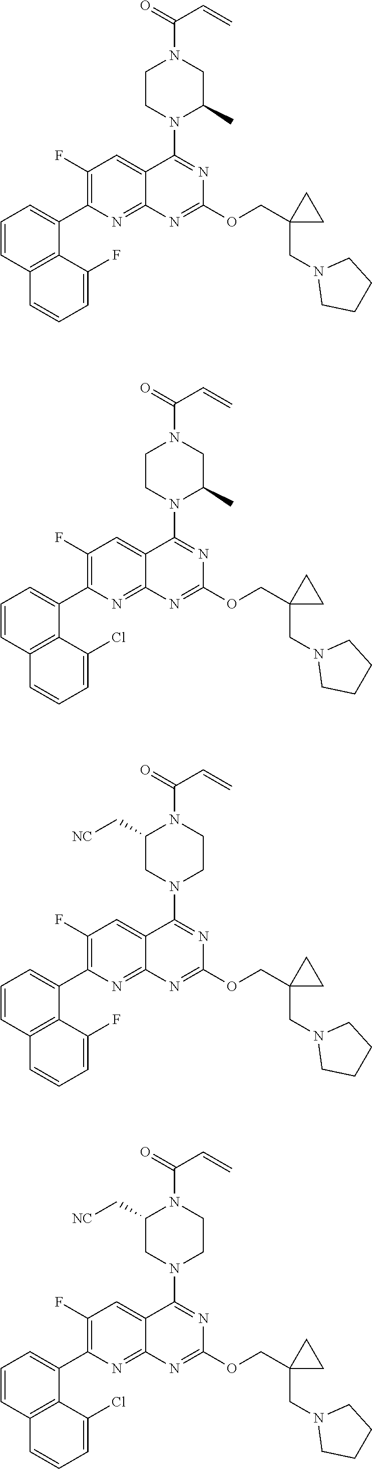 [SMILES: C=CC(=O)N1CCN(c2nc(OCC3(CN4CCCC4)CC3)nc3nc(-c4cccc5cccc(F)c45)c(F)cc23)[C@H](C)C1 ; C=CC(=O)N1CCN(c2nc(OCC3(CN4CCCC4)CC3)nc3nc(-c4cccc5cccc(Cl)c45)c(F)cc23)[C@H](C)C1 ; C=CC(=O)N1CCN(c2nc(OCC3(CN4CCCC4)CC3)nc3nc(-c4cccc5cccc(F)c45)c(F)cc23)C[C@@H]1CC#N ; C=CC(=O)N1CCN(c2nc(OCC3(CN4CCCC4)CC3)nc3nc(-c4cccc5cccc(Cl)c45)c(F)cc23)C[C@@H]1CC#N]

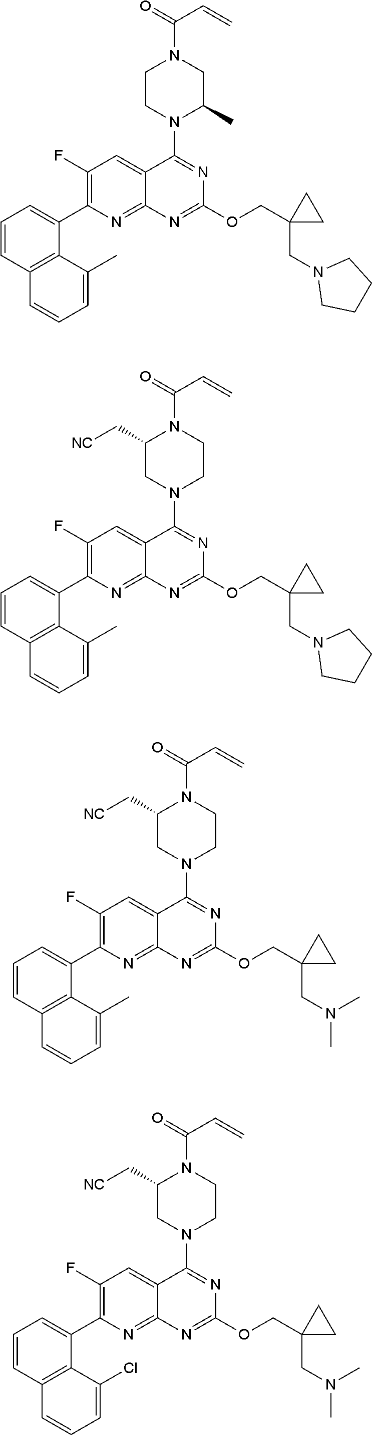 [SMILES: C=CC(=O)N1CCN(c2nc(OCC3(CN4CCCC4)CC3)nc3nc(-c4cccc5cccc(C)c45)c(F)cc23)[C@H](C)C1 ; C=CC(=O)N1CCN(c2nc(OCC3(CN4CCCC4)CC3)nc3nc(-c4cccc5cccc(C)c45)c(F)cc23)C[C@@H]1CC#N ; C=CC(=O)N1CCN(c2nc(OCC3(CN(C)C)CC3)nc3nc(-c4cccc5cccc(C)c45)c(F)cc23)C[C@@H]1CC#N ; C=CC(=O)N1CCN(c2nc(OCC3(CN(C)C)CC3)nc3nc(-c4cccc5cccc(Cl)c45)c(F)cc23)C[C@@H]1CC#N]

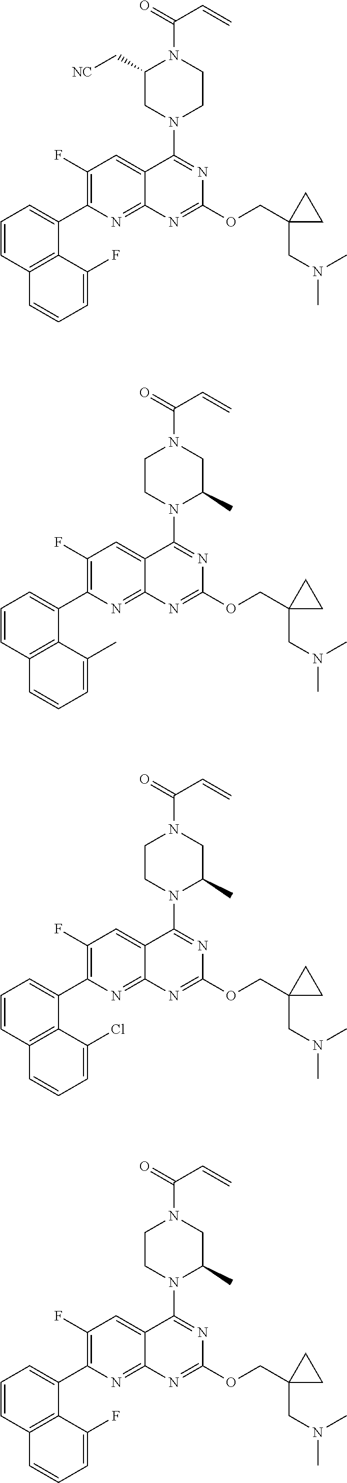 [SMILES: C=CC(=O)N1CCN(c2nc(OCC3(CN(C)C)CC3)nc3nc(-c4cccc5cccc(F)c45)c(F)cc23)C[C@@H]1CC#N ; C=CC(=O)N1CCN(c2nc(OCC3(CN(C)C)CC3)nc3nc(-c4cccc5cccc(C)c45)c(F)cc23)[C@H](C)C1 ; C=CC(=O)N1CCN(c2nc(OCC3(CN(C)C)CC3)nc3nc(-c4cccc5cccc(Cl)c45)c(F)cc23)[C@H](C)C1 ; C=CC(=O)N1CCN(c2nc(OCC3(CN(C)C)CC3)nc3nc(-c4cccc5cccc(F)c45)c(F)cc23)[C@H](C)C1]

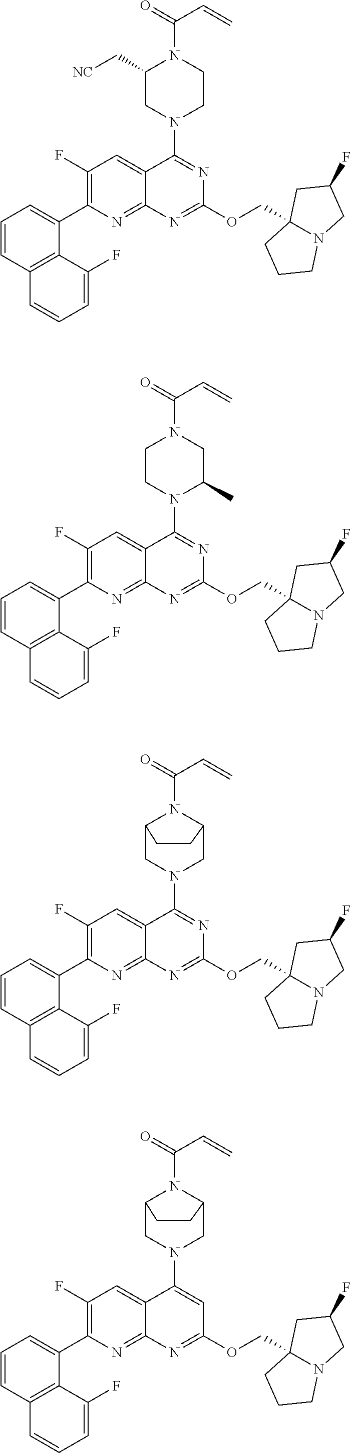 [SMILES: C=CC(=O)N1CCN(c2nc(OC[C@@]34CCCN3C[C@H](F)C4)nc3nc(-c4cccc5cccc(F)c45)c(F)cc23)C[C@@H]1CC#N ; C=CC(=O)N1CCN(c2nc(OC[C@@]34CCCN3C[C@H](F)C4)nc3nc(-c4cccc5cccc(F)c45)c(F)cc23)[C@H](C)C1 ; C=CC(=O)N1C2CCC1CN(c1nc(OC[C@@]34CCCN3C[C@H](F)C4)nc3nc(-c4cccc5cccc(F)c45)c(F)cc13)C2 ; C=CC(=O)N1C2CCC1CN(c1cc(OC[C@@]34CCCN3C[C@H](F)C4)nc3nc(-c4cccc5cccc(F)c45)c(F)cc13)C2]

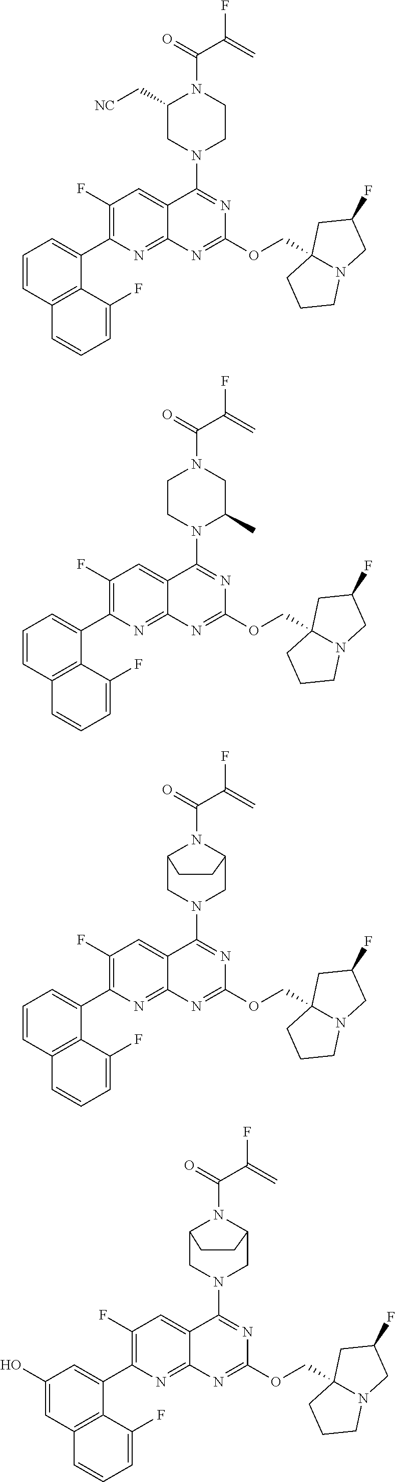 [SMILES: C=C(F)C(=O)N1CCN(c2nc(OC[C@@]34CCCN3C[C@H](F)C4)nc3nc(-c4cccc5cccc(F)c45)c(F)cc23)C[C@@H]1CC#N ; C=C(F)C(=O)N1CCN(c2nc(OC[C@@]34CCCN3C[C@H](F)C4)nc3nc(-c4cccc5cccc(F)c45)c(F)cc23)[C@H](C)C1 ; C=C(F)C(=O)N1C2CCC1CN(c1nc(OC[C@@]34CCCN3C[C@H](F)C4)nc3nc(-c4cccc5cccc(F)c45)c(F)cc13)C2 ; C=C(F)C(=O)N1C2CCC1CN(c1nc(OC[C@@]34CCCN3C[C@H](F)C4)nc3nc(-c4cc(O)cc5cccc(F)c45)c(F)cc13)C2]

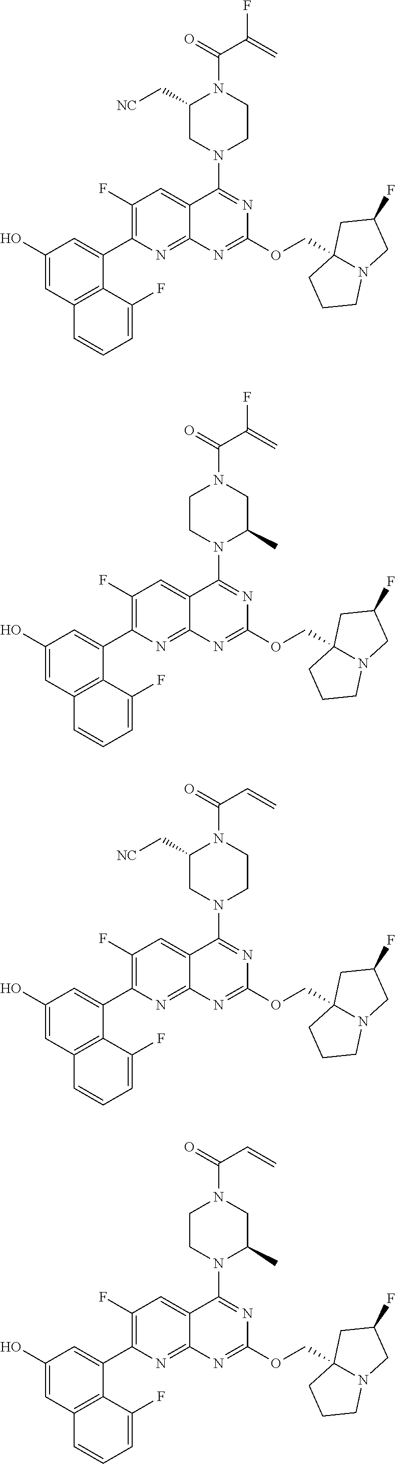 [SMILES: C=C(F)C(=O)N1CCN(c2nc(OC[C@@]34CCCN3C[C@H](F)C4)nc3nc(-c4cc(O)cc5cccc(F)c45)c(F)cc23)C[C@@H]1CC#N ; C=C(F)C(=O)N1CCN(c2nc(OC[C@@]34CCCN3C[C@H](F)C4)nc3nc(-c4cc(O)cc5cccc(F)c45)c(F)cc23)[C@H](C)C1 ; C=CC(=O)N1CCN(c2nc(OC[C@@]34CCCN3C[C@H](F)C4)nc3nc(-c4cc(O)cc5cccc(F)c45)c(F)cc23)C[C@@H]1CC#N ; C=CC(=O)N1CCN(c2nc(OC[C@@]34CCCN3C[C@H](F)C4)nc3nc(-c4cc(O)cc5cccc(F)c45)c(F)cc23)[C@H](C)C1]

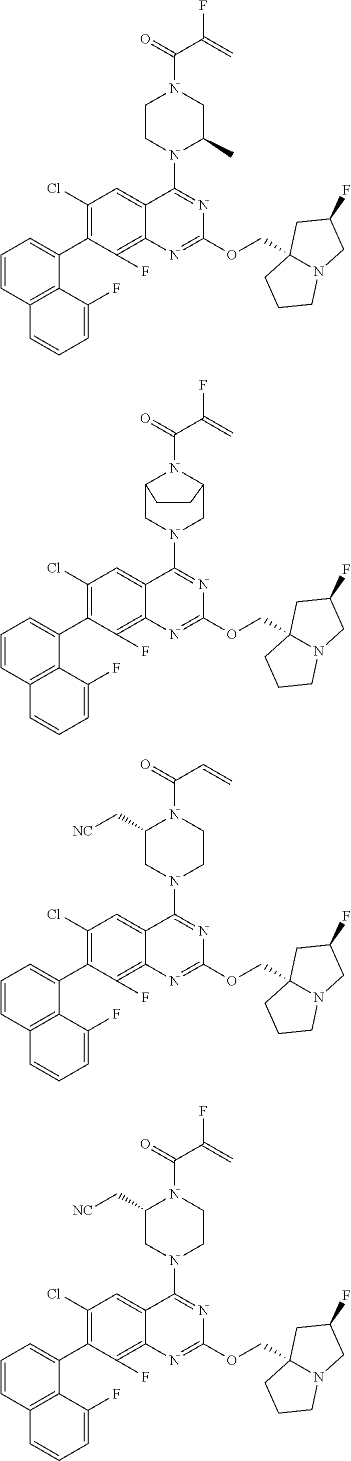 [SMILES: C=C(F)C(=O)N1CCN(c2nc(OC[C@@]34CCCN3C[C@H](F)C4)nc3c(F)c(-c4cccc5cccc(F)c45)c(Cl)cc23)[C@H](C)C1 ; C=C(F)C(=O)N1C2CCC1CN(c1nc(OC[C@@]34CCCN3C[C@H](F)C4)nc3c(F)c(-c4cccc5cccc(F)c45)c(Cl)cc13)C2 ; C=CC(=O)N1CCN(c2nc(OC[C@@]34CCCN3C[C@H](F)C4)nc3c(F)c(-c4cccc5cccc(F)c45)c(Cl)cc23)C[C@@H]1CC#N ; C=C(F)C(=O)N1CCN(c2nc(OC[C@@]34CCCN3C[C@H](F)C4)nc3c(F)c(-c4cccc5cccc(F)c45)c(Cl)cc23)C[C@@H]1CC#N]

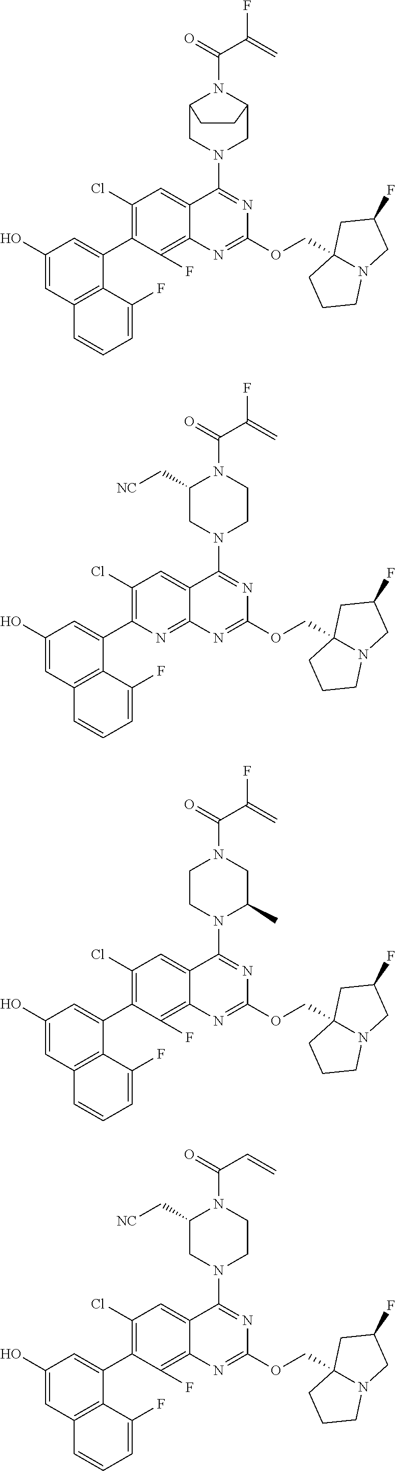 [SMILES: C=C(F)C(=O)N1C2CCC1CN(c1nc(OC[C@@]34CCCN3C[C@H](F)C4)nc3c(F)c(-c4cc(O)cc5cccc(F)c45)c(Cl)cc13)C2 ; C=C(F)C(=O)N1CCN(c2nc(OC[C@@]34CCCN3C[C@H](F)C4)nc3nc(-c4cc(O)cc5cccc(F)c45)c(Cl)cc23)C[C@@H]1CC#N ; C=C(F)C(=O)N1CCN(c2nc(OC[C@@]34CCCN3C[C@H](F)C4)nc3c(F)c(-c4cc(O)cc5cccc(F)c45)c(Cl)cc23)[C@H](C)C1 ; C=CC(=O)N1CCN(c2nc(OC[C@@]34CCCN3C[C@H](F)C4)nc3c(F)c(-c4cc(O)cc5cccc(F)c45)c(Cl)cc23)C[C@@H]1CC#N]

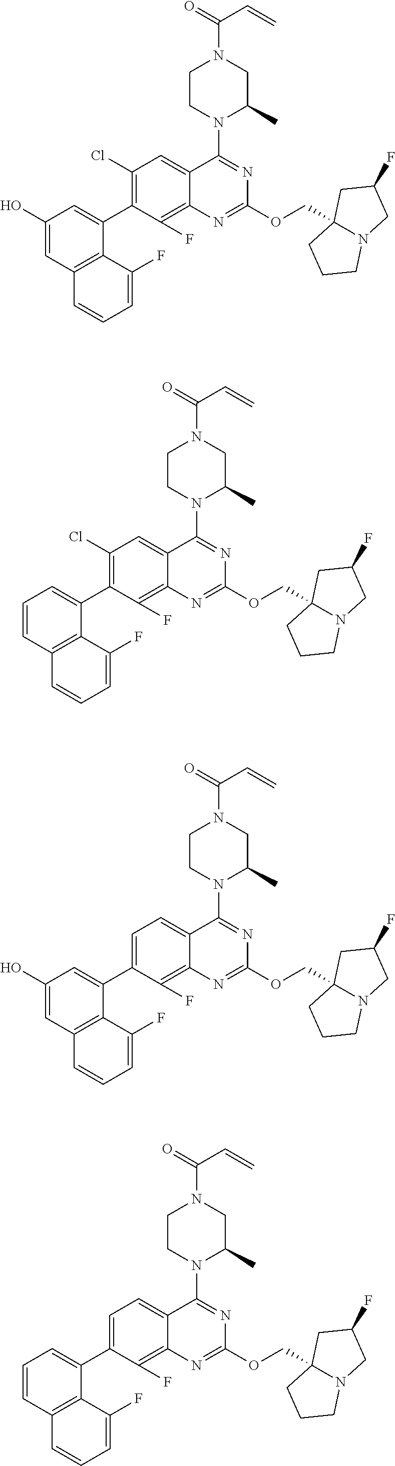 [SMILES: C=CC(=O)N1CCN(c2nc(OC[C@@]34CCCN3C[C@H](F)C4)nc3c(F)c(-c4cc(O)cc5cccc(F)c45)c(Cl)cc23)[C@H](C)C1 ; C=CC(=O)N1CCN(c2nc(OC[C@@]34CCCN3C[C@H](F)C4)nc3c(F)c(-c4cccc5cccc(F)c45)c(Cl)cc23)[C@H](C)C1 ; C=CC(=O)N1CCN(c2nc(OC[C@@]34CCCN3C[C@H](F)C4)nc3c(F)c(-c4cc(O)cc5cccc(F)c45)ccc23)[C@H](C)C1 ; C=CC(=O)N1CCN(c2nc(OC[C@@]34CCCN3C[C@H](F)C4)nc3c(F)c(-c4cccc5cccc(F)c45)ccc23)[C@H](C)C1]

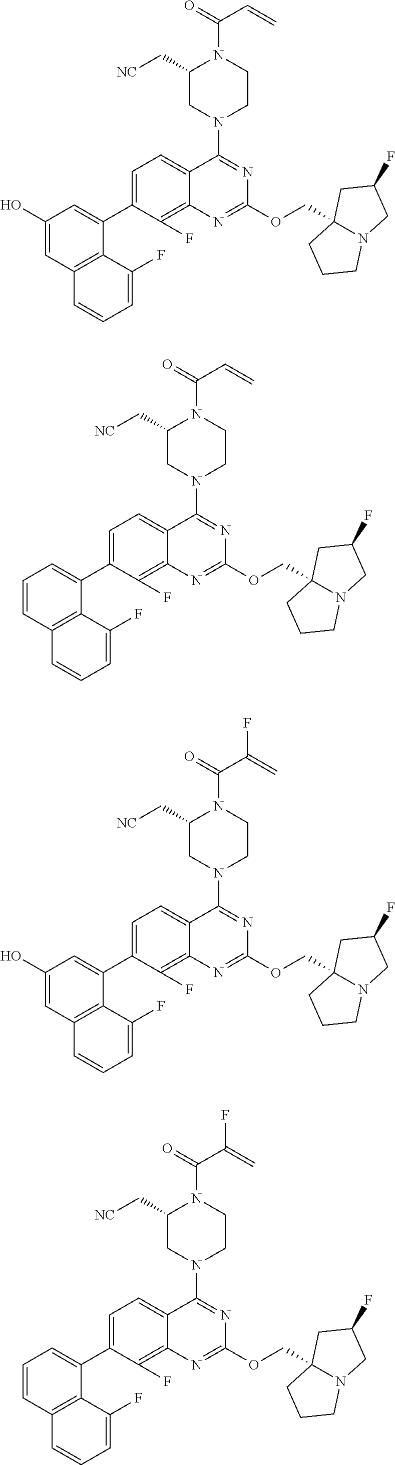 [SMILES: C=CC(=O)N1CCN(c2nc(OC[C@@]34CCCN3C[C@H](F)C4)nc3c(F)c(-c4cc(O)cc5cccc(F)c45)ccc23)C[C@@H]1CC#N ; C=CC(=O)N1CCN(c2nc(OC[C@@]34CCCN3C[C@H](F)C4)nc3c(F)c(-c4cccc5cccc(F)c45)ccc23)C[C@@H]1CC#N ; C=C(F)C(=O)N1CCN(c2nc(OC[C@@]34CCCN3C[C@H](F)C4)nc3c(F)c(-c4cc(O)cc5cccc(F)c45)ccc23)C[C@@H]1CC#N ; C=C(F)C(=O)N1CCN(c2nc(OC[C@@]34CCCN3C[C@H](F)C4)nc3c(F)c(-c4cccc5cccc(F)c45)ccc23)C[C@@H]1CC#N]

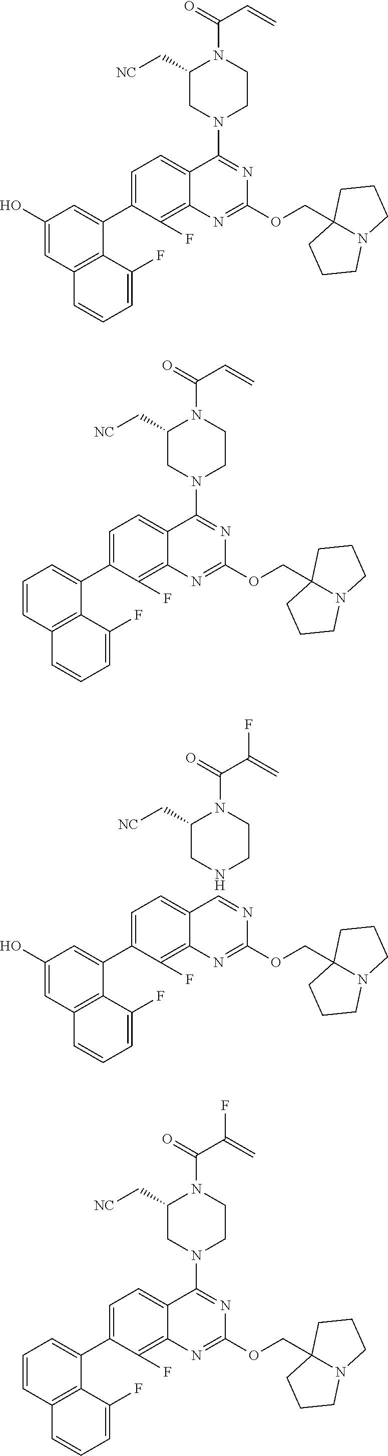

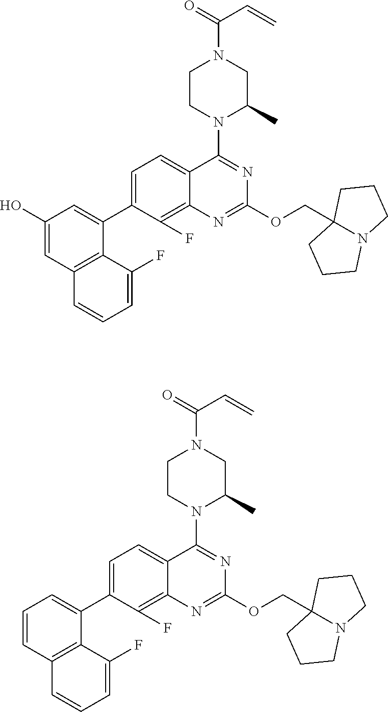 [SMILES: C=CC(=O)N1CCN(c2nc(OCC34CCCN3CCC4)nc3c(F)c(-c4cc(O)cc5cccc(F)c45)ccc23)[C@H](C)C1 ; C=CC(=O)N1CCN(c2nc(OCC34CCCN3CCC4)nc3c(F)c(-c4cccc5cccc(F)c45)ccc23)[C@H](C)C1]

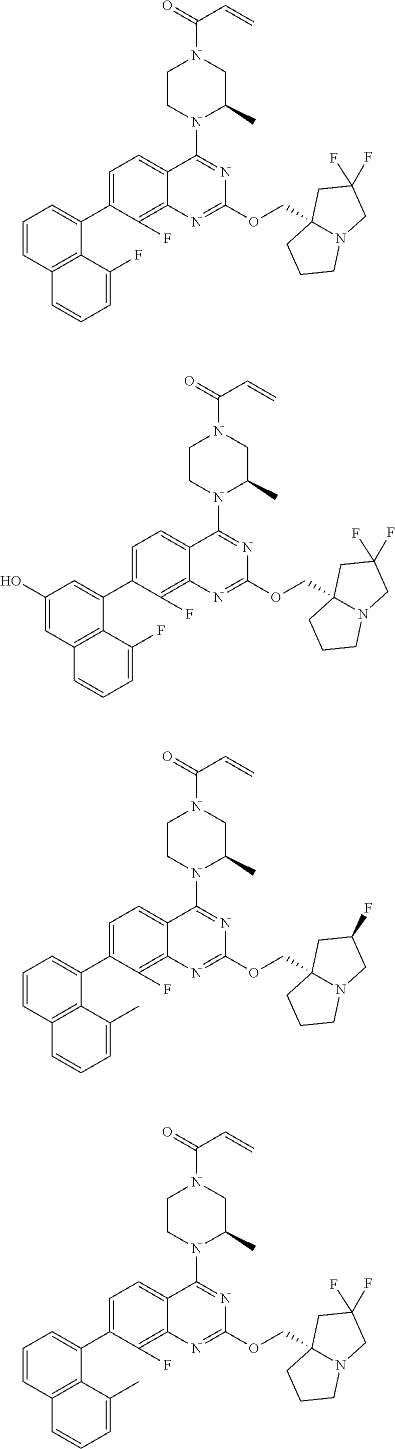 [SMILES: C=CC(=O)N1CCN(c2nc(OC[C@@]34CCCN3CC(F)(F)C4)nc3c(F)c(-c4cccc5cccc(F)c45)ccc23)[C@H](C)C1 ; C=CC(=O)N1CCN(c2nc(OC[C@@]34CCCN3CC(F)(F)C4)nc3c(F)c(-c4cc(O)cc5cccc(F)c45)ccc23)[C@H](C)C1 ; C=CC(=O)N1CCN(c2nc(OC[C@@]34CCCN3C[C@H](F)C4)nc3c(F)c(-c4cccc5cccc(C)c45)ccc23)[C@H](C)C1 ; C=CC(=O)N1CCN(c2nc(OC[C@@]34CCCN3CC(F)(F)C4)nc3c(F)c(-c4cccc5cccc(C)c45)ccc23)[C@H](C)C1]

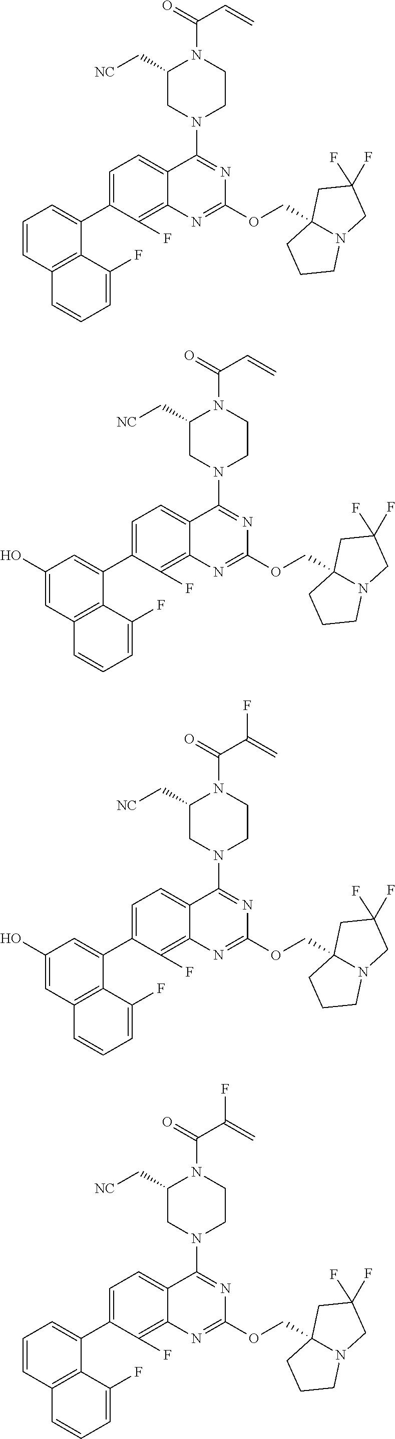 [SMILES: C=CC(=O)N1CCN(c2nc(OC[C@@]34CCCN3CC(F)(F)C4)nc3c(F)c(-c4cccc5cccc(F)c45)ccc23)C[C@@H]1CC#N ; C=CC(=O)N1CCN(c2nc(OC[C@@]34CCCN3CC(F)(F)C4)nc3c(F)c(-c4cc(O)cc5cccc(F)c45)ccc23)C[C@@H]1CC#N ; C=C(F)C(=O)N1CCN(c2nc(OC[C@@]34CCCN3CC(F)(F)C4)nc3c(F)c(-c4cc(O)cc5cccc(F)c45)ccc23)C[C@@H]1CC#N ; C=C(F)C(=O)N1CCN(c2nc(OC[C@@]34CCCN3CC(F)(F)C4)nc3c(F)c(-c4cccc5cccc(F)c45)ccc23)C[C@@H]1CC#N]

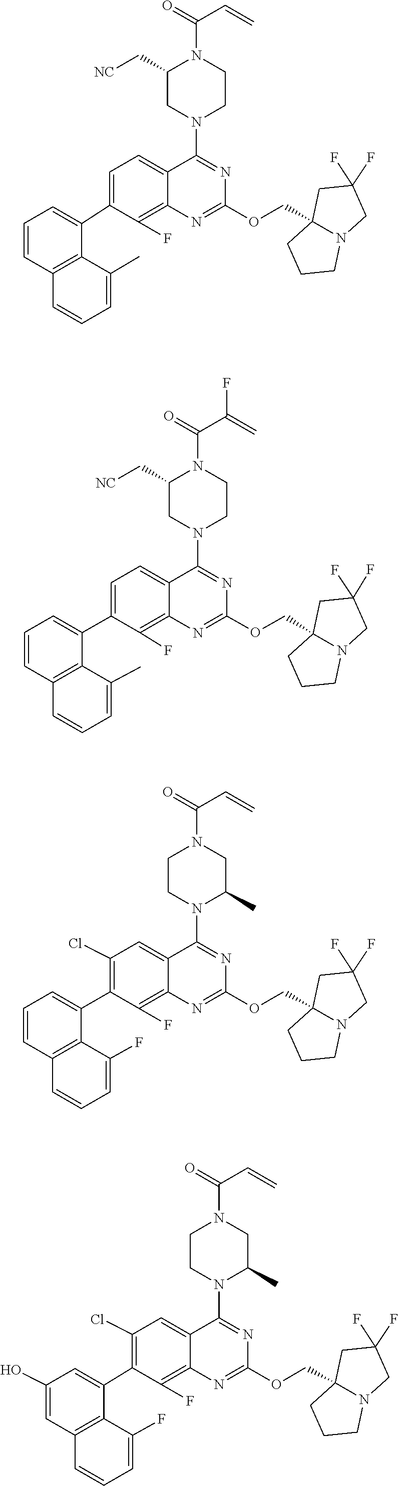 [SMILES: C=CC(=O)N1CCN(c2nc(OC[C@@]34CCCN3CC(F)(F)C4)nc3c(F)c(-c4cccc5cccc(C)c45)ccc23)C[C@@H]1CC#N ; C=C(F)C(=O)N1CCN(c2nc(OC[C@@]34CCCN3CC(F)(F)C4)nc3c(F)c(-c4cccc5cccc(C)c45)ccc23)C[C@@H]1CC#N ; C=CC(=O)N1CCN(c2nc(OC[C@@]34CCCN3CC(F)(F)C4)nc3c(F)c(-c4cccc5cccc(F)c45)c(Cl)cc23)[C@H](C)C1 ; C=CC(=O)N1CCN(c2nc(OC[C@@]34CCCN3CC(F)(F)C4)nc3c(F)c(-c4cc(O)cc5cccc(F)c45)c(Cl)cc23)[C@H](C)C1]

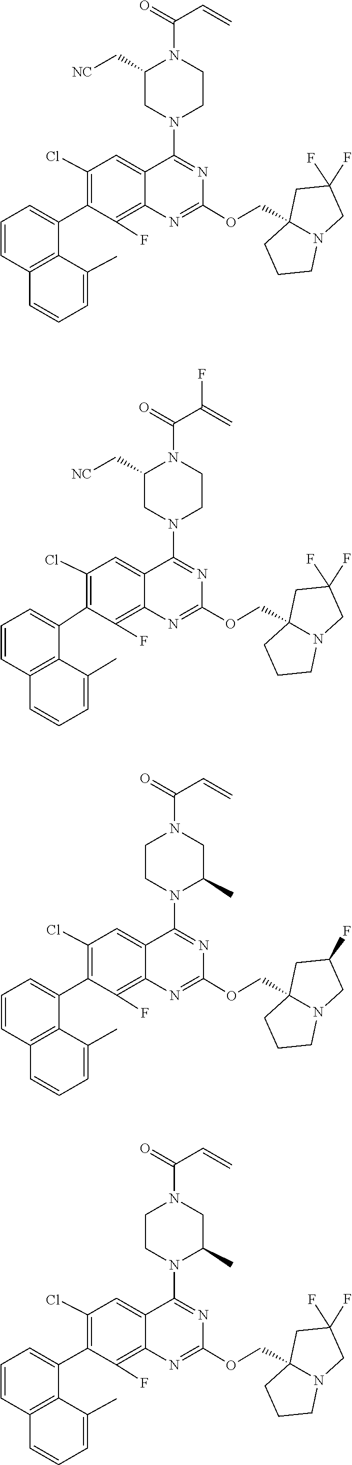 [SMILES: C=CC(=O)N1CCN(c2nc(OC[C@@]34CCCN3CC(F)(F)C4)nc3c(F)c(-c4cccc5cccc(C)c45)c(Cl)cc23)C[C@@H]1CC#N ; C=C(F)C(=O)N1CCN(c2nc(OC[C@@]34CCCN3CC(F)(F)C4)nc3c(F)c(-c4cccc5cccc(C)c45)c(Cl)cc23)C[C@@H]1CC#N ; C=CC(=O)N1CCN(c2nc(OC[C@@]34CCCN3C[C@H](F)C4)nc3c(F)c(-c4cccc5cccc(C)c45)c(Cl)cc23)[C@H](C)C1 ; C=CC(=O)N1CCN(c2nc(OC[C@@]34CCCN3CC(F)(F)C4)nc3c(F)c(-c4cccc5cccc(C)c45)c(Cl)cc23)[C@H](C)C1]

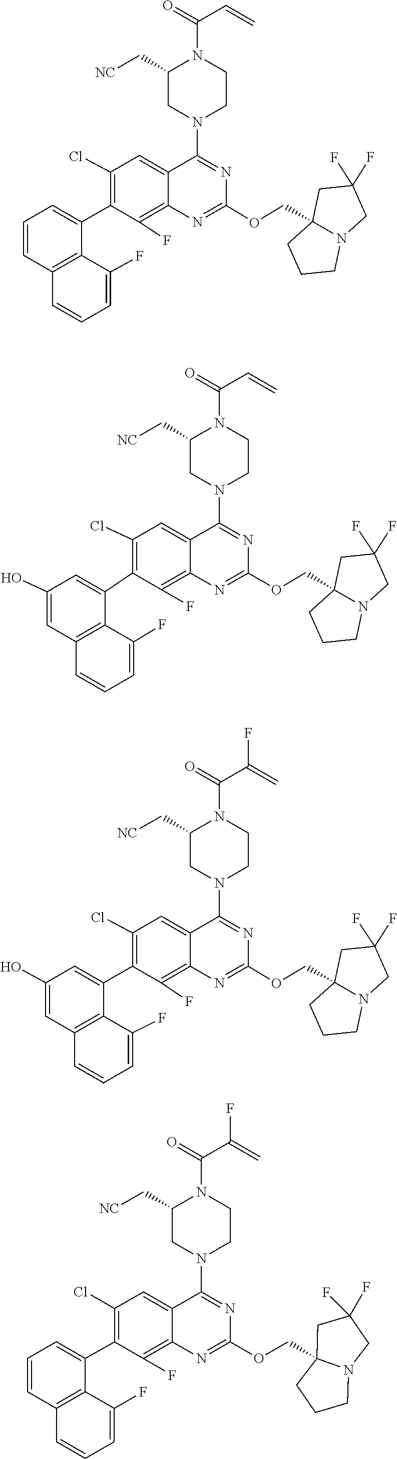 [SMILES: C=CC(=O)N1CCN(c2nc(OC[C@@]34CCCN3CC(F)(F)C4)nc3c(F)c(-c4cccc5cccc(F)c45)c(Cl)cc23)C[C@@H]1CC#N ; C=CC(=O)N1CCN(c2nc(OC[C@@]34CCCN3CC(F)(F)C4)nc3c(F)c(-c4cc(O)cc5cccc(F)c45)c(Cl)cc23)C[C@@H]1CC#N ; C=C(F)C(=O)N1CCN(c2nc(OC[C@@]34CCCN3CC(F)(F)C4)nc3c(F)c(-c4cc(O)cc5cccc(F)c45)c(Cl)cc23)C[C@@H]1CC#N ; C=C(F)C(=O)N1CCN(c2nc(OC[C@@]34CCCN3CC(F)(F)C4)nc3c(F)c(-c4cccc5cccc(F)c45)c(Cl)cc23)C[C@@H]1CC#N]

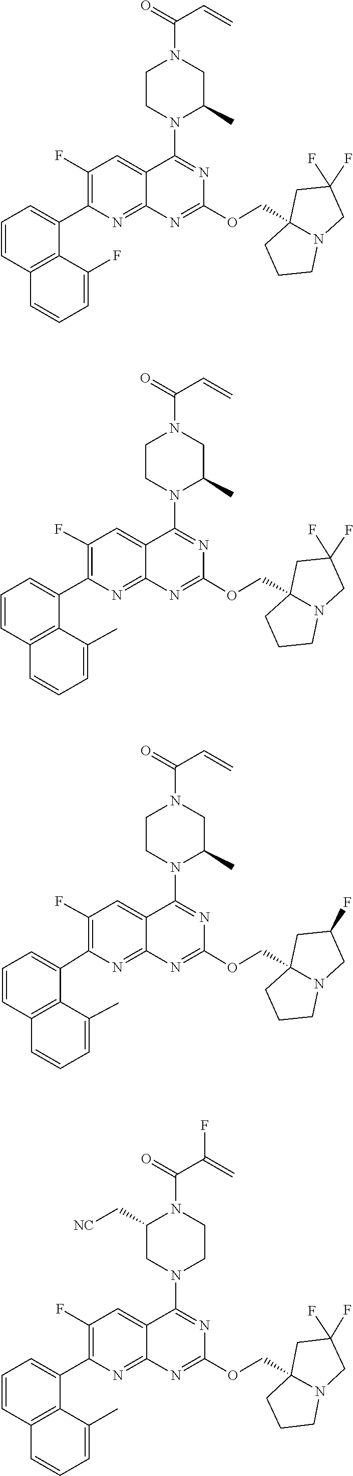 [SMILES: C=CC(=O)N1CCN(c2nc(OC[C@@]34CCCN3CC(F)(F)C4)nc3nc(-c4cccc5cccc(F)c45)c(F)cc23)[C@H](C)C1 ; C=CC(=O)N1CCN(c2nc(OC[C@@]34CCCN3CC(F)(F)C4)nc3nc(-c4cccc5cccc(C)c45)c(F)cc23)[C@H](C)C1 ; C=CC(=O)N1CCN(c2nc(OC[C@@]34CCCN3C[C@H](F)C4)nc3nc(-c4cccc5cccc(C)c45)c(F)cc23)[C@H](C)C1 ; C=C(F)C(=O)N1CCN(c2nc(OC[C@@]34CCCN3CC(F)(F)C4)nc3nc(-c4cccc5cccc(C)c45)c(F)cc23)C[C@@H]1CC#N]

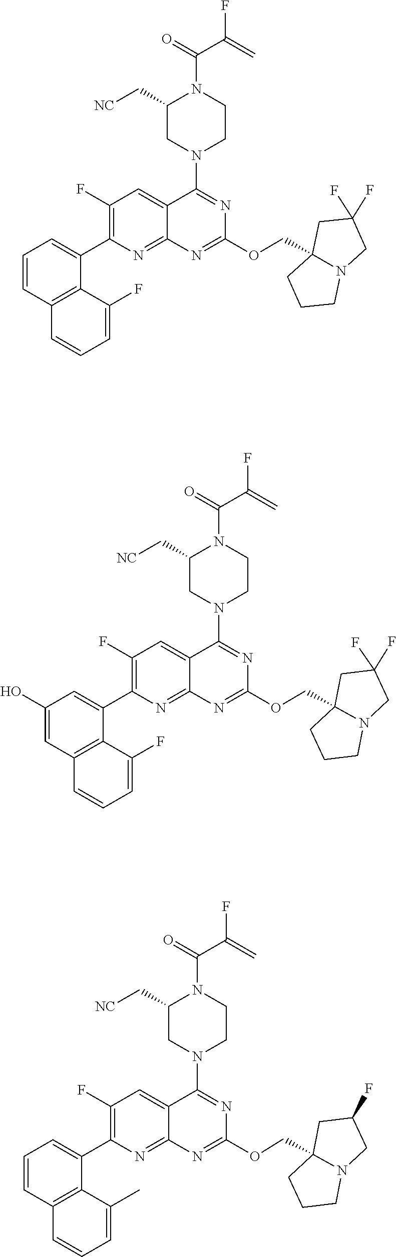 [SMILES: C=C(F)C(=O)N1CCN(c2nc(OC[C@@]34CCCN3CC(F)(F)C4)nc3nc(-c4cccc5cccc(F)c45)c(F)cc23)C[C@@H]1CC#N ; C=C(F)C(=O)N1CCN(c2nc(OC[C@@]34CCCN3CC(F)(F)C4)nc3nc(-c4cc(O)cc5cccc(F)c45)c(F)cc23)C[C@@H]1CC#N ; C=C(F)C(=O)N1CCN(c2nc(OC[C@@]34CCCN3C[C@H](F)C4)nc3nc(-c4cccc5cccc(C)c45)c(F)cc23)C[C@@H]1CC#N]

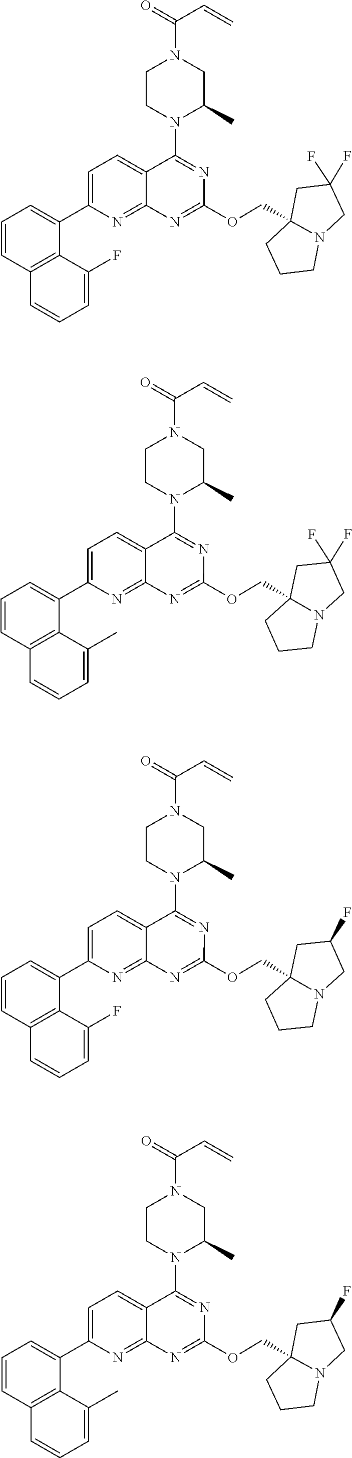 [SMILES: C=CC(=O)N1CCN(c2nc(OC[C@@]34CCCN3CC(F)(F)C4)nc3nc(-c4cccc5cccc(F)c45)ccc23)[C@H](C)C1 ; C=CC(=O)N1CCN(c2nc(OC[C@@]34CCCN3CC(F)(F)C4)nc3nc(-c4cccc5cccc(C)c45)ccc23)[C@H](C)C1 ; C=CC(=O)N1CCN(c2nc(OC[C@@]34CCCN3C[C@H](F)C4)nc3nc(-c4cccc5cccc(F)c45)ccc23)[C@H](C)C1 ; C=CC(=O)N1CCN(c2nc(OC[C@@]34CCCN3C[C@H](F)C4)nc3nc(-c4cccc5cccc(C)c45)ccc23)[C@H](C)C1]

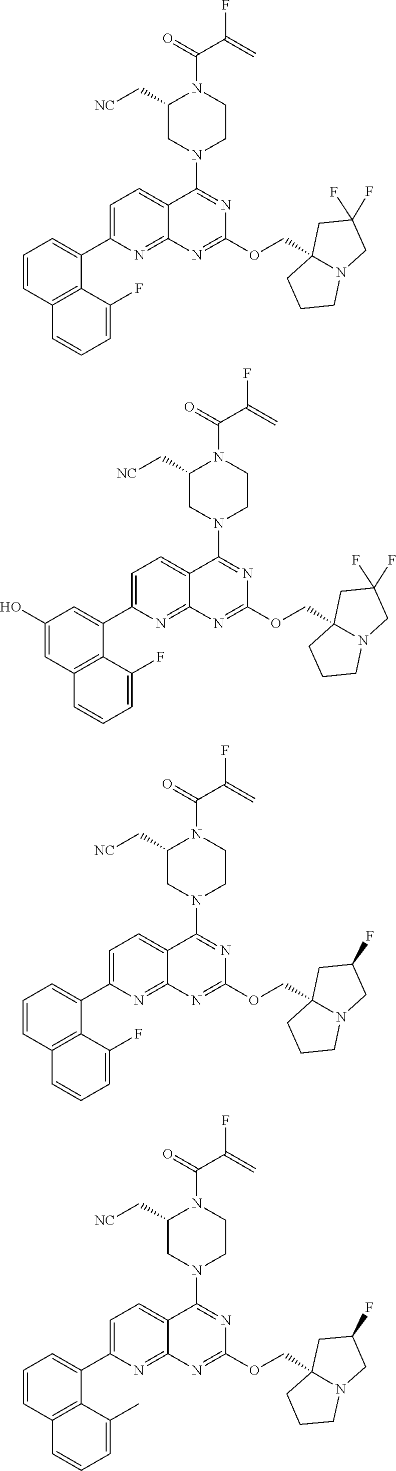 [SMILES: C=C(F)C(=O)N1CCN(c2nc(OC[C@@]34CCCN3CC(F)(F)C4)nc3nc(-c4cccc5cccc(F)c45)ccc23)C[C@@H]1CC#N ; C=C(F)C(=O)N1CCN(c2nc(OC[C@@]34CCCN3CC(F)(F)C4)nc3nc(-c4cc(O)cc5cccc(F)c45)ccc23)C[C@@H]1CC#N ; C=C(F)C(=O)N1CCN(c2nc(OC[C@@]34CCCN3C[C@H](F)C4)nc3nc(-c4cccc5cccc(F)c45)ccc23)C[C@@H]1CC#N ; C=C(F)C(=O)N1CCN(c2nc(OC[C@@]34CCCN3C[C@H](F)C4)nc3nc(-c4cccc5cccc(C)c45)ccc23)C[C@@H]1CC#N]

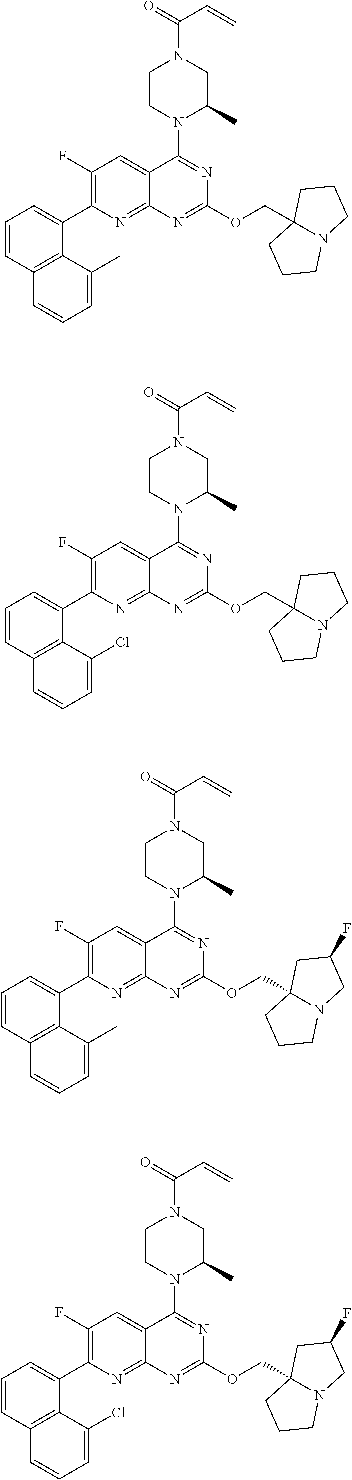 [SMILES: C=CC(=O)N1CCN(c2nc(OCC34CCCN3CCC4)nc3nc(-c4cccc5cccc(C)c45)c(F)cc23)[C@H](C)C1 ; C=CC(=O)N1CCN(c2nc(OCC34CCCN3CCC4)nc3nc(-c4cccc5cccc(Cl)c45)c(F)cc23)[C@H](C)C1 ; C=CC(=O)N1CCN(c2nc(OC[C@@]34CCCN3C[C@H](F)C4)nc3nc(-c4cccc5cccc(C)c45)c(F)cc23)[C@H](C)C1 ; C=CC(=O)N1CCN(c2nc(OC[C@@]34CCCN3C[C@H](F)C4)nc3nc(-c4cccc5cccc(Cl)c45)c(F)cc23)[C@H](C)C1]

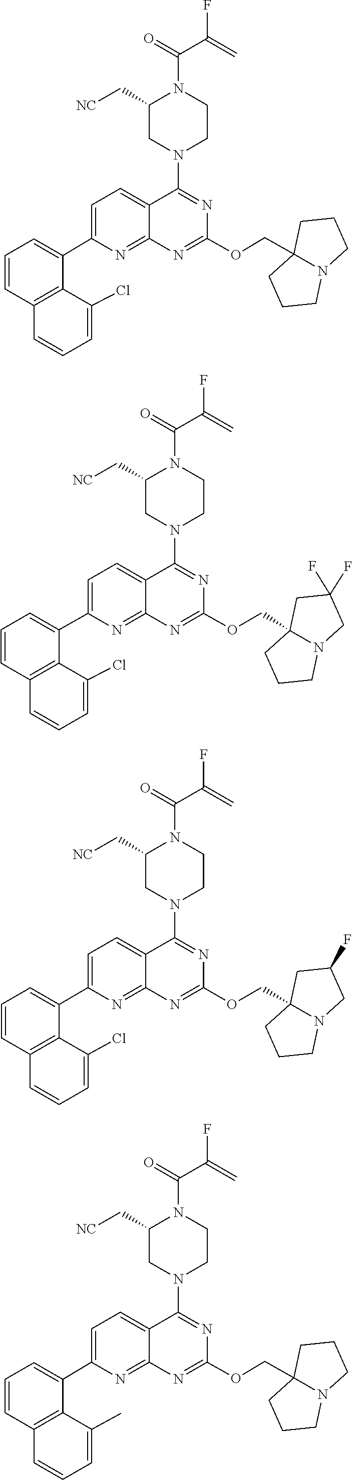 [SMILES: C=C(F)C(=O)N1CCN(c2nc(OCC34CCCN3CCC4)nc3nc(-c4cccc5cccc(Cl)c45)ccc23)C[C@@H]1CC#N ; C=C(F)C(=O)N1CCN(c2nc(OC[C@@]34CCCN3CC(F)(F)C4)nc3nc(-c4cccc5cccc(Cl)c45)ccc23)C[C@@H]1CC#N ; C=C(F)C(=O)N1CCN(c2nc(OC[C@@]34CCCN3C[C@H](F)C4)nc3nc(-c4cccc5cccc(Cl)c45)ccc23)C[C@@H]1CC#N ; C=C(F)C(=O)N1CCN(c2nc(OCC34CCCN3CCC4)nc3nc(-c4cccc5cccc(C)c45)ccc23)C[C@@H]1CC#N]

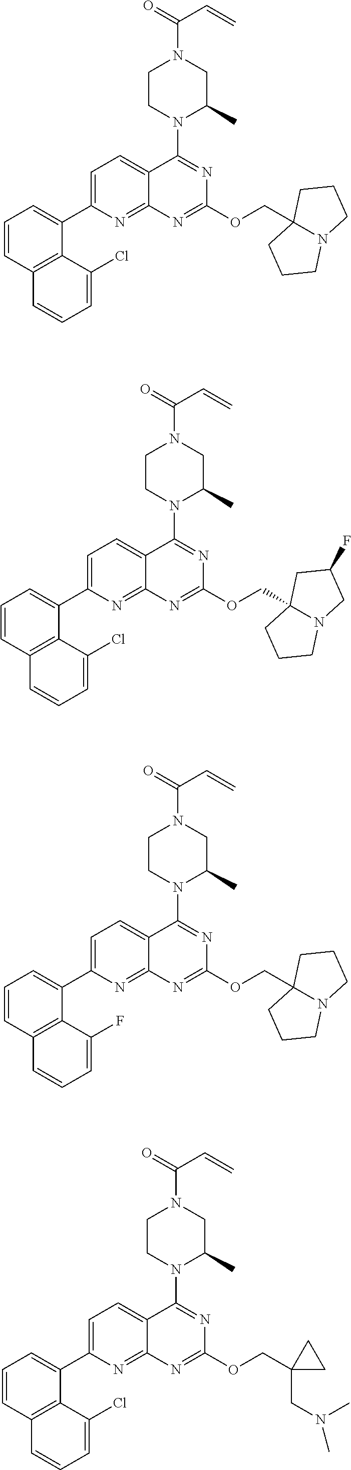 [SMILES: C=CC(=O)N1CCN(c2nc(OCC34CCCN3CCC4)nc3nc(-c4cccc5cccc(Cl)c45)ccc23)[C@H](C)C1 ; C=CC(=O)N1CCN(c2nc(OC[C@@]34CCCN3C[C@H](F)C4)nc3nc(-c4cccc5cccc(Cl)c45)ccc23)[C@H](C)C1 ; C=CC(=O)N1CCN(c2nc(OCC34CCCN3CCC4)nc3nc(-c4cccc5cccc(F)c45)ccc23)[C@H](C)C1 ; C=CC(=O)N1CCN(c2nc(OCC3(CN(C)C)CC3)nc3nc(-c4cccc5cccc(Cl)c45)ccc23)[C@H](C)C1]

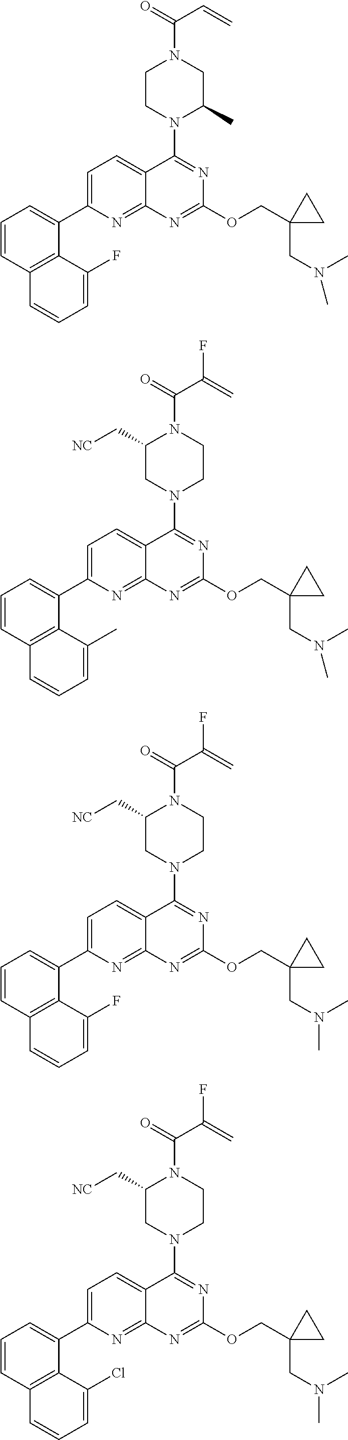 [SMILES: C=CC(=O)N1CCN(c2nc(OCC3(CN(C)C)CC3)nc3nc(-c4cccc5cccc(F)c45)ccc23)[C@H](C)C1 ; C=C(F)C(=O)N1CCN(c2nc(OCC3(CN(C)C)CC3)nc3nc(-c4cccc5cccc(C)c45)ccc23)C[C@@H]1CC#N ; C=C(F)C(=O)N1CCN(c2nc(OCC3(CN(C)C)CC3)nc3nc(-c4cccc5cccc(F)c45)ccc23)C[C@@H]1CC#N ; C=C(F)C(=O)N1CCN(c2nc(OCC3(CN(C)C)CC3)nc3nc(-c4cccc5cccc(Cl)c45)ccc23)C[C@@H]1CC#N]

7. A pharmaceutical composition, characterized in that, the pharmaceutical composition comprises (1) the compound according to claim 1; and (2) a pharmaceutically acceptable carrier.

8. A use of the compound according to claim 1, characterized in that, for the preparation of a pharmaceutical composition, the pharmaceutical composition is used for: (i) preventing and/or treating tumors; (ii) inhibiting or reversing tumor multidrug resistance to anti-tumor drugs; (iii) inhibiting P-glycoprotein; (iv) enhancing anti-tumor activity of anti-tumor drugs; and/or (v) inhibiting KRAS^{G12C} mutant protein-related cancers in drugs application.
Preferably, the cancer is selected from the group consisting of blood cancer, lung cancer, pancreatic cancer, colon cancer, rectal cancer, colorectal cancer, oral cancer; the blood cancer is selected from acute myeloid leukemia or acute lymphoblastic leukemia, The lung cancer is selected from non-small cell lung cancer or small cell lung cancer.

9. The use according to claim 8, wherein the tumor comprises a tumor with multidrug resistance to antitumor drugs.
